(19) United States
(12) Patent Application Publication
(10) Pub. No.: US 2026/0079150 A1
(43) Pub. Date: Mar. 19, 2026

(54) METHODS AND APPARATUS FOR SIMULTANEOUS IDENTIFICATION AND QUANTIFICATION OF A MICROBE

(71) Applicant: Oxbridge Clinical Inc., Boston, MA (US)

(72) Inventors: Giulio Deangeli, Boston, MA (US); Cristiano Peron, Boston, MA (US)

(73) Assignee: Oxbridge Clinical Inc., Boston, MA (US)

(21) Appl. No.: 18/886,214

(22) Filed: Sep. 16, 2024

(51) Int. Cl. G01N 33/487

(52) U.S. Cl. CPC G01N 33/48721

(57) ABSTRACT

A method for simultaneous identification and quantification of a microbe includes accepting, by a nanopore reader, a sample including at least a microbe, detecting, by a detector, a signal as a function of the at least a microbe, wherein the at least a microbe is translocated from a first flow cell to a second flow cell through at least a nanopore, correlating, by a control unit, a first attribute and a second attribute of the detected signal, identifying, by the control unit, one or more types of microbe as a function of the correlation, classifying, by the control unit, a plurality of events within the detected signal based on the identified one or more types of microbe, and quantifying, by the control unit, at least one type of microbe of the identified one or more types of microbe as a function of the classified plurality of events.

### FIELD OF THE INVENTION

The present invention generally relates to the field of identification and quantification of microbes. In particular, the present invention is directed to apparatus and methods for simultaneous identification and quantification of a microbe using nanopores.

### BACKGROUND

Clinical decisions often require an evaluation of antimicrobial resistance using an antibiogram to determine whether a microbe, such as bacteria, is sensitive or resistant to one or more antibiotics. Evaluation of antimicrobial resistance often involves culturing, isolating, and identifying a microbe before administering one or more antibiotics and monitoring its response thereto. As a result, development of an antibiogram requires both an identification and a quantification of one or more microbes. Current technologies are limited to performing an identification step and a quantification step in a sequential manner. Most current tools for germ-specific microbial detection rely on nucleic acid-based or antibody-based technologies, which may be costly, hard to deploy at the point of care, and inherently germ-specific, requiring a test to be performed at least once for each pathogen to be tested. Additionally, development of microbial cultures may take several days or weeks, as it requires a microbe such as a pathogen to undergo repeated reproduction cycles to become identifiable. The natural background of microbiota and/or other impurities also poses an additional challenge to the identification and/or quantification of a microbe of medical interest.

The widely adopted optical density (OD) methods may indirectly approximate the number and size of cells indirectly by detecting changes in light scattering in a bulk sample. However, despite their relative ease of implementation, the accuracy of OD measurements may be inherently hindered by challenges such as low sensitivity and nonlinearity at extreme concentration ranges. Modern bacterial growth monitoring methods such as flow cytometry and microscopy, while having their own merits, are not suitable for time-critical studies which involve frequent time-course measurements, due to their tedious preparation steps, such as fixation and staining, and requirements for a large sample volume, etc. Some of these methods also require relatively high instrumental and maintenance costs. While certain tools or devices such as Coulter counters are capable of quantifying the number, number density, or concentration of a microbe, these tools are unable to identify the microbe, and their uses are accordingly limited to purified microbes only.

Similar challenges also exist for other nonmedical use cases such as preservation of food, cosmetics, or the like.

### SUMMARY OF THE DISCLOSURE

In an aspect, a method for simultaneous identification and quantification of microbial growth is described. Method includes accepting, by a first flow cell, a sample including at least one microbe. Method further includes detecting, by at least a detector, a signal as a function of at least a microbe, wherein the at least a microbe is translocated from a first flow cell to a second flow cell through at least a nanopore. Method further includes correlating, by a control unit, a first attribute and a second attribute of detected signal. Method further includes identifying, by control unit, one or more types of microbe as a function of correlation. Method further includes classifying, by control unit, a plurality of events within detected signal based on identified one or more types of microbe. Method further includes quantifying, by control unit, at least one type of microbe of identified one or more types of microbe as a function of classified plurality of events.

In another aspect, an apparatus for simultaneous identification and quantification of microbial growth is described. Apparatus includes at least a nanopore. Apparatus further includes at least a nanopore reader. Each nanopore reader of at least a nanopore reader includes a plurality of flow cells, wherein at least a flow cell of the plurality of flow cells is configured to accept a sample. Each nanopore reader of at least a nanopore reader further includes at least a detector connected to plurality of flow cells, wherein the at least a detector is configured to detect a signal as a function of at least a translocated microbe from the sample. Apparatus further includes a control unit communicatively connected to at least a detector. Control unit is configured to correlate a first attribute and a second attribute of the detected signal. Control unit is further configured to identify one or more types of microbe as a function of correlation. Control unit is further configured to classify a plurality of events within detected signal based on identified one or more types of microbe. Control unit is further configured to quantify at least one type of microbe of identified one or more types of microbe as a function of classified plurality of events.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic illustration of an exemplary embodiment of an apparatus for identification and quantification of a microbe;

FIGS. 2A-C are exemplary embodiments of nanopores and various parameters related thereto;

FIGS. 3A-D are exemplary embodiments of various designs of flow cells and nanopore readers;

FIG. 4A is an exemplary embodiment of a plurality of nanopores arranged in a line;

FIG. 4B is an exemplary embodiment of a plurality of nanopores arranged in a two-dimensional (2D) matrix;

FIG. 5A is an exemplary embodiment of an extended signal containing a plurality of flat intervals;

FIG. 5B is an exemplary embodiment of an identified event between two flanking flat intervals.

FIG. 5C is an exemplary embodiment of several attributes that describe an identified event;

FIG. 6A is an exemplary embodiment of experimental results for bacterial concentrations measured using Escherichia coli, after 0 h, 1 h, 3 h, and 5 h of incubation in growth media that contain no treatment, a treatment of 10 μg/mL ciprofloxacin, and a treatment of 100 μg/mL ciprofloxacin, respectively;

FIG. 6B is an exemplary embodiment of experimental results for bacterial concentrations measured using Escherichia coli, after 0 h, 2.5 h, and 5 h of incubation in growth media that contain no treatment and a treatment of 1% phenoxyethanol, respectively;

FIGS. 7A-F are exemplary embodiments of correlation plots generated using principal component analysis (PCA) and/or linear discriminant analysis (LDA), based on a first attribute and a second attribute of data; these data are measured from a mixture of spectinomycin-sensitive Escherichia coli and spectinomycin-resistant Salmonella enterica, after 0 h, 2.5 h, and 5 h of incubation in growth media that contain no treatment and a treatment of 50 μg/mL spectinomycin, respectively; associated results of identification are also included;

FIG. 7G is an exemplary embodiment of an experimental protocol used for generating data in FIGS. 7A-F;

FIG. 7H is an exemplary embodiment of experimental results for bacterial concentrations measured based on FIGS. 7A-F;

FIGS. 8A-C are exemplary embodiments of resistive curves collected using Escherichia coli, Moraxella catarrhalis, and Salmonella enterica;

FIG. 8D is an exemplary embodiment of correlation plots between a width attribute and a height attribute based on events in FIGS. 8A-C;

FIG. 8E is an exemplary embodiment of a feature-engineering process that may be used to generate the correlation plot in FIG. 8D;

FIG. 8F is an exemplary embodiment of correlation plots generated using long-short-term memory (LSTM), multi-layer perceptron (MLP), and/or PCA;

FIG. 8G is an exemplary embodiment of a machine-learning architecture, including LSTM, MLP, and/or PCA, that may be used to generate the correlation plot in FIG. 8F;

FIGS. 8H-L are exemplary embodiments of correlation plots generated using LSTM, MLP, a random forest (RF) classifier, PCA, and/or LDA at various certainty cutoffs using data measured from Influenza virus type A and Adenovirus type 5 with overlapping correlations; an associated exemplary embodiment of RF-based machine-learning architecture is also included;

FIG. 9 is a block diagram of an exemplary embodiment of a machine-learning process;

FIG. 10 is a block diagram of an exemplary embodiment of a neural network;

FIG. 11 is a block diagram of an exemplary embodiment of a node of a neural network;

FIGS. 12A-E are exemplary embodiments of workflows for implementing a machine-learning process in identification and/or quantification of a microbe;

FIG. 13 is an exemplary flow diagram illustrating a method for identification and quantification of a microbe; and

FIG. 14 is a block diagram of an exemplary embodiment of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for identification and quantification of a microbe. Apparatus includes one or more nanopores. In one or more embodiments, at least a nanopore may be excavated in a SiNₓ wafer, a silicon oxide wafer, a glass wafer, or a polyimide membrane, among others. In one or more embodiments, at least a first nanopore of a plurality of nanopores may have a first size between 100 nanometers and 20 micrometers, and at least a second nanopore of the plurality of nanopores may have a second size between 100 nanometers and 20 micrometers, wherein the first size is different from the second size. In one or more embodiments, at least a first nanopore of plurality of nanopores may have a first geometry, and at least a second nanopore of the plurality of nanopores may have a second geometry, wherein the first geometry is different from the second geometry. In one or more embodiments, at least a first nanopore of plurality of nanopores may be applied with a first voltage difference along a first longitudinal axis of the at least a first nanopore, and at least a second nanopore of the plurality of nanopores may be applied with a second voltage difference along a second longitudinal axis of the at least a second nanopore, wherein the first voltage difference is different from the second voltage difference. In one or more embodiments, at least a nanopore may include a coating layer such as an aluminum oxide (AlOₓ) layer, a silicon oxide (SiOₓ) layer, a self-assembled monolayer including a fluorinated self-assembled coating layer, among others. In one or more embodiments, plurality of nanopores may be disposed in a line. In one or more embodiments, plurality of nanopores may be disposed in a two-dimensional matrix. In one or more embodiments, plurality of nanopores may be disposed in a three-dimensional matrix.

Apparatus further includes at least a nanopore reader. Each nanopore reader of at least a nanopore reader includes a plurality of flow cells, wherein at least a flow cell of the plurality of flow cells is configured to accept a sample. In one or more embodiments, a first flow cell of plurality of flow cells may be configured to accept a sample, whereas a second flow cell of the plurality of flow cells may be configured to accept a reference. First flow cell may intersect second flow cell at a junction, and at least a nanopore may be located at the junction and connecting between the first flow cell and the second flow cell. Each nanopore reader of at least a nanopore reader further includes at least a detector connected to plurality of flow cells, wherein the at least a detector is configured to detect a signal as a function of at least a translocated microbe from sample. In one or more embodiments, detected signal may include an optical signal such as an absorbance, optical density, fluorescence, or the like. In one or more embodiments, detected signal may include an electrical signal such as a resistive pulse. In one or more embodiments, at least a detector may include an optical sensor such as a photodetector, an optical absorption spectrometer, an optical emission spectrometer, or the like. In one or more embodiments, at least a detector may include a resistive pulse sensor, such as a tunable resistive pulse sensor.

Apparatus further includes a control unit communicatively connected to at least a detector. Control unit is configured to correlate a first attribute and a second attribute of detected signal. In one or more embodiments, correlating first attribute and second attribute of detected signal may include receiving correlation training data including a plurality of exemplary correlations as outputs correlated to a plurality of exemplary signal attributes as inputs. Accordingly, in one or more embodiments, correlating first attribute and second attribute of detected signal may further include iteratively training a correlation machine-learning model using correlation training data. Accordingly, in one or more embodiments, correlating first attribute and second attribute of detected signal may further include correlating the first attribute and the second attribute of the detected signal using trained correlation machine-learning model. In one or more embodiments, correlating first attribute and second attribute may include correlating the first attribute and the second attribute using principal component analysis (PCA) and/or linear discriminant analysis (LDA). Control unit is further configured to identify one or more types of microbes as a function of correlation.

Control unit is further configured to classify a plurality of events within detected signal based on identified one or more types of microbes. In one or more embodiments, classifying plurality of events may include classifying the plurality of events using a binary classification algorithm or a multi-class classification (MCC) algorithm. In one or more embodiments, classifying plurality of events may include receiving classification training data including a plurality of exemplary classes as outputs correlated to a plurality of exemplary events as inputs. Accordingly, in one or more embodiments, classifying plurality of events may further include iteratively training a classification machine-learning model using classification training data. Accordingly, in one or more embodiments, classifying plurality of events may further include classifying the plurality of events using trained classification machine-learning model. In some cases, exemplary events may include events extracted from experimental data collected using one or more purified microbial samples. Accordingly, in some cases, exemplary signal attributes, as described above, may include exemplary signal attributes extracted from such exemplary events. In some cases, classifying plurality of events further may include determining, using classification machine-learning model, a certainty score and filtering the plurality of events as a function of the certainty score. In some cases, classification machine-learning model may include an ensemble of a plurality of classifiers of either the same type or mixed types.

Control unit is further configured to quantify at least one type of microbe of identified one or more types of microbes as a function of classified plurality of events.

Aspects of the present disclosure may be used to provide efficient means for developing antibiograms. Aspects of the present disclosure may be used to provide efficient means for evaluating antimicrobial resistance (AMR) and/or multi-drug resistance (MDR). Aspects of the present disclosure may be used to gauge the efficacy of preservatives and preservation techniques in food and/or cosmetics. Aspects of the present disclosure may be used to characterize and quantify nanoparticles/microparticles of nonliving matter, such as without limitation microplastics, and evaluate their responses (e.g., dissolution or aggregation) to certain processing techniques or treatments, such as without limitation an addition of certain reagents or solvents. Exemplary embodiments illustrating aspects of the present disclosure are described below in the context of several specific examples.

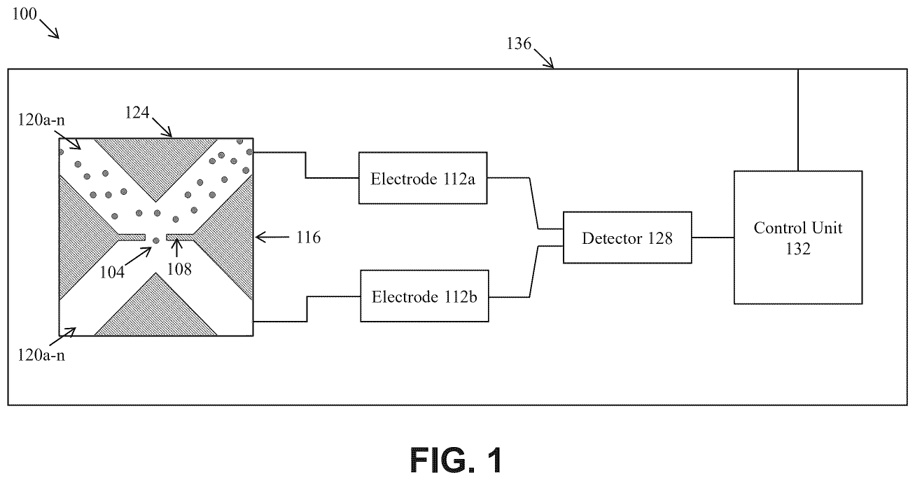

Referring now to FIG. 1, an apparatus 100 for simultaneous identification and quantification of a microbe is illustrated. In one or more embodiments, apparatus 100 may be used for simple detection and quantification of a microbe at a fixed spot. In one or more embodiments, apparatus 100 may be used to evaluate the response of one or more microbes to one or more chemical agents, such as without limitation one or more antibiotics and/or one or more preservatives. In one or more embodiments, apparatus 100 may be used to analyze (i.e., identify and/or quantify) a single type of microbe. In one or more embodiments, apparatus 100 may be used to analyze (i.e., identify and/or quantify) two or more types of microbe. In one or more embodiments, apparatus 100 may be used to monitor microbial growth, such as via repeated measurements as a function of time. For the purposes of this disclosure, “microbial growth” is an increase in number of one or more types of microbes as they replicate. In some cases, microbial growth may include bacterial infection in a clinical context. For the purposes of this disclosure, a “microbe” is an organism of microscopic size, which may exist in its single-celled form or as a colony of cells (except for viruses that do not have cellular structures) and may potentially function as a pathogen and infect a host to result in one or more symptoms. Microbes may include viruses such as without limitation Influenzavirus, Parainfluenzavirus, Rhinovirus, Adenovirus, or Respiratory Syncytial Virus. Microbes may include bacteria such as without limitation Escherichia coli, Salmonella enterica, or Streptococcus pyogenes. Microbes may include fungi such as without limitation Histoplasma capsulatum or Rhizopus oryzae, among others. Microbes may include other types of microorganisms not disclosed herein but deemed relevant to apparatus 100 by a person of ordinary skill in the art upon reviewing the entirety of this disclosure. Microbes may be of a variety of sizes; for example, viruses are typically between 20 nanometers and 200 nanometers, bacteria are typically between 1 micrometer and 10 micrometers, whereas fungi are typically between 10 micrometers and 100 micrometers.

With continued reference to FIG. 1, Accordingly, apparatus 100 may be used to evaluate the impact of a drug on a microbe, such as antimicrobial resistance (AMR) and/or multiple-drug resistance (MDR). In one or more embodiments, such drug may include an antibiotic such as a broad-spectrum antibiotic, as described in further detail in this disclosure. For the purposes of this disclosure, “antimicrobial resistance (AMR)” is an ability of a microbe to withstand the effects of antimicrobial drugs that were once effective in treating infections caused by these organisms. When a microbe develops AMR, standard treatments may become ineffective, infections caused by such microbe may persist, and its risk of spreading to others may increase. AMR may occur through various mechanisms, including without limitation genetic mutations, acquisition of resistance genes via horizontal gene transfer, and the selective pressure exerted by the overuse or misuse of antimicrobials. In some cases, bacteria may randomly develop genomic mutations that make them resistant to antibiotics, and such mutations may become positively selected when patients undergo antibiotic treatments, especially when the treatment is not carried out for the entire prescribed duration. In some cases, horizontal gene transfer may occur through plasmids, which are extra-genomic circular DNA pieces with genes that convey resistance to a certain antibiotic. Many species of bacteria may pass on these plasmids to each other, a process known as bacterial conjugation.

With continued reference to FIG. 1, for the purposes of this disclosure, “multiple-drug resistance (MDR)” is a specific form of AMR where a microorganism becomes resistant to multiple classes of antimicrobial agents, making it difficult or even impossible to treat with conventional therapies. MDR is a consequence of widespread AMR. MDR is particularly concerning in bacteria, where pathogens may be resistant to multiple antibiotics, such as methicillin-resistant Staphylococcus aureus (MRSA) or multidrug-resistant Mycobacterium tuberculosis (MDR-TB), may pose significant challenges in clinical settings. MDR may arise through various mechanisms, such as without limitation the production of enzymes that degrade antibiotics, changes in the bacterial cell wall that prevent drug entry, changes/mutations in genes that encode proteins targeted by a drug, or the use of efflux pumps that expel antibiotics from the cell. As a nonlimiting example, an antibiotic may target and deactivate a certain enzyme, whereas a mutation in a genetic sequence that encodes this enzyme may render the enzyme incapable of being acted upon by the antibiotic. In some cases, certain bacteria may become resistant to all available antibiotics, resulting in a case of pan-resistance.

With continued reference to FIG. 1, both AMR and MDR are major public health concerns globally, as they lead to longer hospital stays, higher medical costs, and increased mortality. As a nonlimiting example, Streptococcus pneumoniae, while being responsible for over 2 million infections every year, was recently shown to be resistant to clinically relevant antibiotics in over 30% patients. As another nonlimiting example, pneumococcal pneumonia may cause about 150,000 people in the U.S. to be hospitalized each year, killing about 5-7%, or 1 in 20 of those infected. The death rate associated with pneumococcal pneumonia is even higher among adults aged 65 years and older and people with certain medical conditions or other risk factors.

With continued reference to FIG. 1, for some common pathogens, may be crucial to know their resistance status, as such status may substantially change a therapeutic indication. As an example, one of the most common bacteria in human healthy skin, which is also a major cause of soft tissue infection, is Staphylococcus aureus. Wild-type Staphylococcus aureus is naturally sensitive to penicillin derivatives (beta-lactams). However, 2% of the population, and up to 75% or more in a hospital setting, may carry a version of Staphylococcus aureus which has acquired resistance to beta-lactams (i.e., methicillin-resistant Staphylococcus aureus or MRSA). In case of a soft-tissue infection from MRSA, it may be imperative to utilize non-beta-lactam antibiotics, and such choice can carry life-threatening importance.

With continued reference to FIG. 1, apparatus 100 may be used to generate an antibiogram. For the purposes of this disclosure, an “antibiogram” is a report of data that contains the results of antimicrobial susceptibility testing for a specific set of bacterial strains isolated from clinical samples. An antibiogram summarizes the effectiveness of various antibiotics against these bacteria by showing which antibiotics the bacteria are susceptible to, resistant to, or intermediate in response. This information is typically presented in a tabular format, with bacterial species listed on one axis and antibiotics on the other. The data within an antibiogram are often derived from culture and sensitivity tests performed on clinical specimens such as blood, urine, sputum, or wound swabs, where bacteria are grown in a growth medium, isolated, and exposed to various antibiotics to assess their susceptibility. Antibiograms are commonly requested by clinicians in a variety of contexts. Some may be non-urgent, such as without limitation a patient with non-life-threatening skin ulcer. Some may be urgent, such as a patient with sepsis while being immunocompromised. An antibiogram is usually generated by microbiology laboratories within hospitals or healthcare institutions and may be specific to a certain department, hospital unit, or patient population. A set of antibiograms may be used to guide healthcare providers in selecting an appropriate antibiotic therapy for individual patients, based on the local patterns of bacterial resistance. Additionally, antibiograms may be used in public health and hospital settings to monitor trends in antibiotic resistance over time, helping to inform infection control strategies and antibiotic stewardship programs.

With continued reference to FIG. 1, an antibiogram may typically be generated via two steps. Step 1 may include creating a microbial culture, where a clinical sample may first be grown on several bacterial growth media; accordingly, bacterial strains are isolated therefrom and the pathogen causing the disease may be identified. At step 2, antibiotics may be administered to assess whether the bacterial colony isolated from the microbial culture in step 1 dies as a result.

With continued reference to FIG. 1, the current antibiogram technique often suffers from two major drawbacks. First, the procedure is slow due to the need to isolate a bacterial strain. For extremely fast-growing bacteria such as Escherichia coli, this step may take about 1 day. For more ordinary bacteria, this step may usually take 2-3 days. For slowly growing bacteria, this step may take more than a month. Second, the procedure is costly, as it requires full lab infrastructure, with sterile facilities and bacterial hood. Antibiograms for non-urgent cases may take up to several weeks due to limited availability of lab facilities. In contrast, apparatus 100 described herein is capable of delivering an antibiogram in just a few hours (and in some cases, within 30 minutes), without the need for lab facilities and qualified personnel.

With continued reference to FIG. 1, it is worth noting that the use cases of apparatus 100 may not be limited to bacteria-related applications and/or antibiograms only. Instead, apparatus 100 may be used to evaluate any sensitivity assay pertaining to fungi, viruses, and/or the like, that is deemed suitable by a person of ordinary skill in the art, upon reviewing the entirety of this disclosure. As a nonlimiting example, apparatus 100 may be used to identify and quantify one or more viruses and/or their responses to certain agents of treatment. As another nonlimiting example, apparatus 100 may be used for a simple quantification of Influenzavirus on eukaryotic cell cultures and/or in the supernatant thereof (i.e., a spot quantification). As another nonlimiting example, apparatus 100 may be used to quantify the number of copies of a virus in the supernatant of a cell culture over time. As another nonlimiting example, apparatus 100 may be used to evaluate the response of a virus against one or more external substances. It is worth noting that, since viruses cannot replicate by themselves and may only replicate in a host cell, the resistance mechanisms of viruses are generally more subtle than the resistance mechanisms of bacteria. As a nonlimiting example, the resistance mechanism of a virus may involve tricking an immune system or an interferon system. As another nonlimiting example, the resistance mechanism of a virus may involve modifying the receptor(s) of the virus in order to enter a specific type of cell. These mechanisms may depend on both the type of host and the type of virus and may thus be more challenging to study in vitro. Accordingly, application of apparatus 100 for identification and quantification viruses may likely be implemented as a spot detection/quantification.

With continued reference to FIG. 1, outside a clinical context, apparatus 100 may be used to evaluate the efficacies of preservatives in a variety of consumer products that are subject to stringent criteria on their sterility. Manufacturers often need to periodically show that their products, in their specific formulation, do not allow for bacterial growth up to a certain legal maximum; for example, in the case of cosmetics such as toothpastes, regulatory requirements are governed by ISO-11930.1. One of the most widely used preservatives is phenoxyethanol. For this reason, according to the European Union Cosmetics Regulation (EC) n. 1223/2009, phenoxyethanol is authorized as a preservative in cosmetic formulations at a maximum concentration of 1%. For limitations similar to the case of antibiograms described above, these tests may also be time-consuming and costly and may take about one month to complete. Apparatus 100 described herein is capable of substantially cutting down both the costs and the duration of such tests.

With continued reference to FIG. 1, apparatus 100 includes at least a nanopore 104. For the purposes of this disclosure, a “nanopore” is a hollow cavity or channel with two orifices/open ends and a lateral dimension on a nanometer-to-micrometer scale. In other words, for the purposes of this disclosure, the word “nanopore” may represent both nanopores and micropores. For the purposes of this disclosure, a “longitudinal” direction of a nanopore is the direction that extends from one opening to the other, whereas a “lateral” direction is the direction that is transverse and perpendicular to the longitudinal direction. For the purposes of this disclosure, a “lateral dimension” of a nanopore is the size of the nanopore along its lateral direction. In one or more embodiments, nanopore 104 may have a lateral dimension (e.g., a diameter) between 100 nanometers and 20 micrometers. As nonlimiting examples, nanopore 104 may have a lateral dimension between 100 nanometers and 200 nanometers, between 200 nanometers and 300 nanometers, between 300 nanometers and 400 nanometers, between 400 nanometers and 500 nanometers, between 500 nanometers and 600 nanometers, between 600 nanometers and 700 nanometers, between 700 nanometers and 800 nanometers, between 800 nanometers and 900 nanometers, between 900 nanometers and 1 micrometer, between 1 micrometer and 2 micrometers, between 2 micrometers and 5 micrometers, between 5 micrometers and 10 micrometers, between 10 micrometers and 15 micrometers, or between 15 micrometers and 20 micrometers.

With continued reference to FIG. 1, nanopore 104 may be constructed within a thin-layer matrix 108 of any durable solid-state matrix or material considered suitable (e.g., both mechanically robust during processing and electrically non-conductive) by a person of ordinary skill in the art upon reviewing the entirety of this disclosure. Additionally, nanopore 104 may be constructed in any suitable geometries and/or combination of geometries. Additionally, nanopore 104 may adopt any shape at its open ends, as recognized by a person of ordinary skill in the art upon reviewing the entirety of this disclosure, such as circular, elliptical, polygonal, or the like. Additionally, nanopore 104 may have any surface treatment, coating, and/or functionalization at its interior wall, as recognized by a person of ordinary skill in the art upon reviewing the entirety of this disclosure.

With continued reference to FIG. 1, in one or more embodiments, two potentials/voltages may be applied, for example and without limitation, through two electrodes 112a-b, at the two open ends of nanopore 104, resulting in a voltage difference across longitudinal axis of nanopore 104. Exemplary voltage differences may include without limitation ±20 V, ±18 V, ±16 V, ±14 V, ±12 V, ±10 V, ±8 V, ±6 V, ±4 V, ±2 V, ±1.5 V, ±1 V, or ±0.5 V, ±0.4 V, ±0.3 V, ±0.2 V, ±0.1 V, ±90 mV, ±80 mV, ±70 mV, ±60 mV, ±50 mV, ±40 mV, ±30 mV, ±20 mV, ±10 mV, ±5 mV, etc.

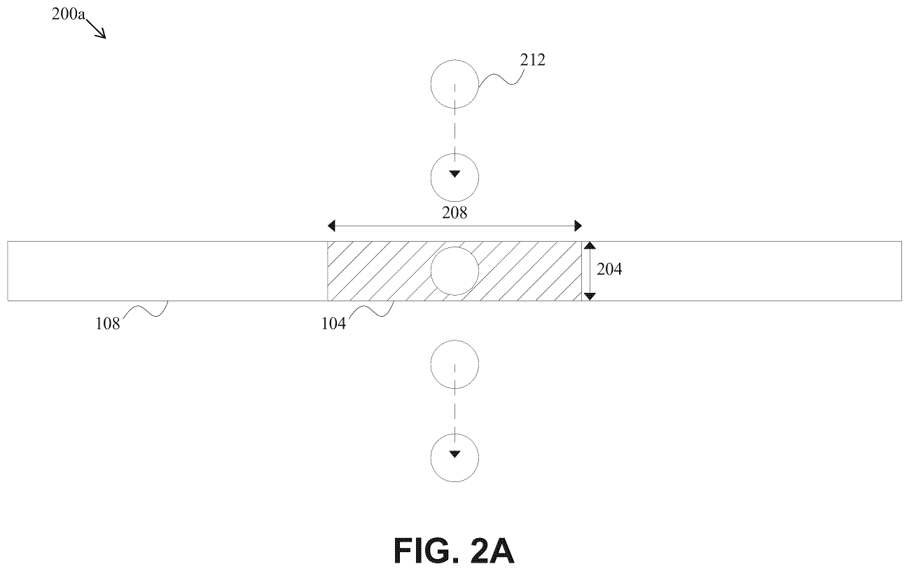

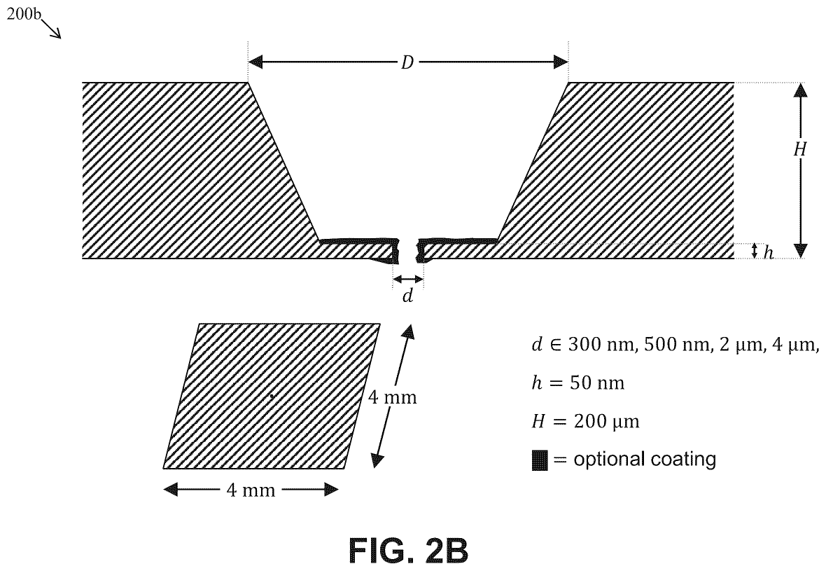

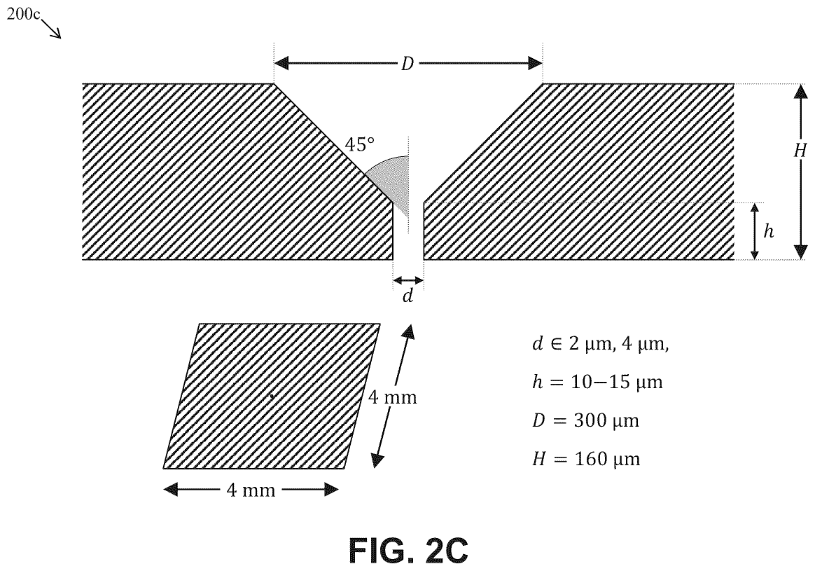

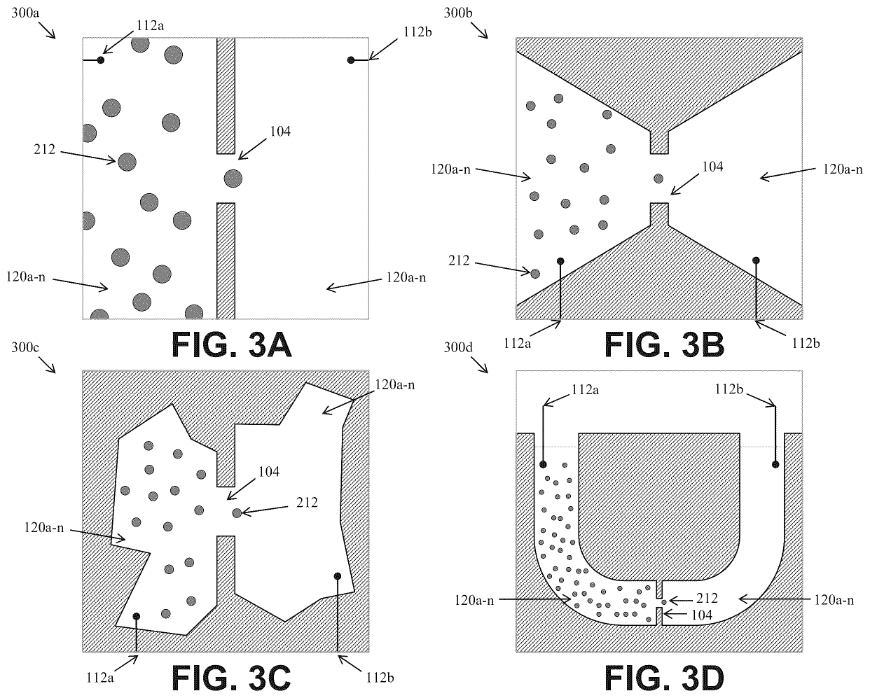

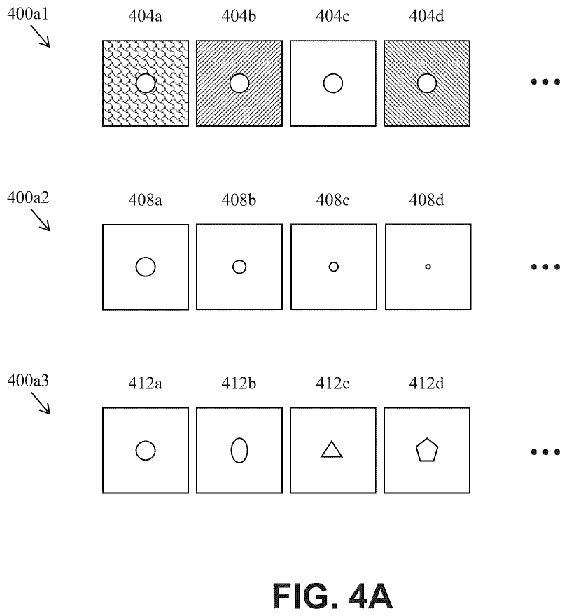

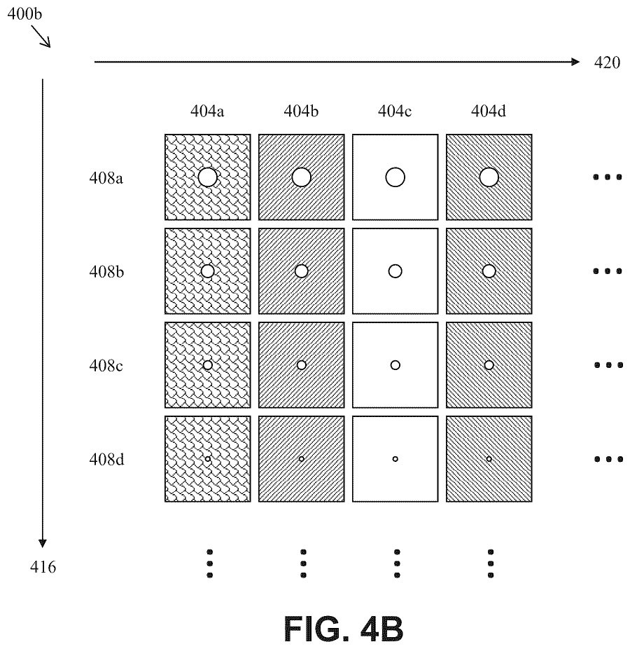

With continued reference to FIG. 1, the choice of size, shape, geometry, and/or applied potential of nanopore 104, either individually or in combination, may impose certain constraints on the size, shape, geometry, surface charge, and/or type of microbe that may pass through the nanopore 104. In some cases, microbes may have various sizes, shapes, and/or surface charges that interact with nanopore 104 in different manners, e.g., as a function of the size, geometry, voltage difference, and/or coating materials of the nanopore, as described in detail below in this disclosure. As a nonlimiting example, a small microbe may be able to pass through nanopores 104 of all sizes, whereas larger microbes may selectively pass through nanopores 104 of larger sizes that are beyond a certain threshold. As another nonlimiting example, a microbe with a positively charged surface may selectively pass through nanopores 104 with negative electrostatic charges, a microbe with a negatively charged surface may selectively pass through nanopores 104 with positive electrostatic charges, and a microbe with an electrically neutral surface may pass through nanopores 104 of all types/polarities of electrostatic charges. Additional details will be provided below in this disclosure with reference to FIGS. 2A-C, FIGS. 3A-D, and FIGS. 4A-B.

With continued reference to FIG. 1, apparatus 100 includes at least a nanopore reader 116. For the purposes of this disclosure, a “nanopore reader” is a device capable of monitoring the chemical and/or biological species that pass nanopore 104. Each nanopore reader 116 of the at least a nanopore reader 116 includes a plurality of flow cells 120a-n, wherein at least a flow cell 120a-n within the plurality of flow cells 120a-n is configured to accept a sample. For the purposes of this disclosure, a “flow cell” is a specialized device used in various scientific and industrial applications to analyze or manipulate fluids as they flow through a confined space. A flow cell typically includes a chamber or channel through which a liquid or gas flows, allowing for real-time observation, measurement, or reaction of a fluid. Flow cell 120a-n may be constructed from any suitable material recognized by a person of ordinary skill in the art upon reviewing the entirety of this disclosure; suitable materials may include, for example and without limitation, quartz, glass, polyethylene, polypropylene, poly(methyl methacrylate) (PMMA), polyimides (PIs), polydimethylsiloxane (PDMS), or the like. Similarly, flow cell 120a-n may be manufactured using any manufacturing process deemed suitable by a person of ordinary skill in the art, upon reviewing the entirety of this disclosure. As nonlimiting examples, flow cell 120a-n may be manufactured using 3D-printing techniques such as without limitation Selective Laser Sintering (SLS), Digital Light Processing (DLP), Fused Deposition Modelling (FDM), Multi Jet Fusion (MJF), binder jetting, stereolithography (SLA), and/or the like. As nonlimiting examples, suitable materials to be used for SLA may include without limitation standard resins, tough resins, flexible resins, high-temperature resins, castable resins, biocompatible resins, engineering resins, ceramic-filled resins, or the like. Additionally, and/or alternatively, suitable materials for SLA may include without limitation a polypropylene (PP)-like material, a polycarbonate (PC)-like material, and/or an acrylonitrile butadiene styrene (ABS)-like material, among others. Additionally, and/or alternatively, suitable materials for SLA may include a nontransparent material of high optical density (e.g., white, gray, black, red, and/or blue, among others). Additionally, and/or alternatively, suitable materials for SLA may include a clear/translucent material. As a nonlimiting example, suitable materials for SLA may Somos PerFORM, which is a thermoplastic filled with ceramic.

With continued reference to FIG. 1, alternatively, and/or additionally, flow cell 120a-n may be constructed in any shape or design that's deemed suitable for apparatus 100 by a person of ordinary skill in the art upon reviewing the entirety of this disclosure. FIG. 1 shows nanopore reader 116 and flow cells 120a-n in an X-shaped design. Details regarding alternative designs of flow cell 120a-n will be provided below in this disclosure when discussing FIGS. 3A-D. In some cases, thin-layer matrix 108 containing nanopore 104 may be fused with one more flow cells 120a-n in one piece, or otherwise in contact or integrated with one or more flow cells 120a-n. Sample may come from any source where at least a microbe of interest may be found. As a nonlimiting example, samples may be collected from clinical samples such as respiratory swab, bronchoalveolar lavage, blood, saliva, and urine. As another nonlimiting example, samples may be collected from water, air, biofilms, veterinary samples, food (e.g., milk), sewage, soil, or the like. As another nonlimiting example, samples may be collected from an air-conditioning system for detection of bacteria such as Legionella pneumophila. As another nonlimiting example, samples may be collected from air filtration/purification/ventilation systems such as in poultry sheds. In one or more embodiments, a first flow cell 120a-n within plurality of flow cells 120a-n may be configured to accept a sample, whereas a second flow cell 120a-n within plurality of flow cells 120a-n may be configured to accept a reference. Accordingly, the side at which first flow cell 120a-n is disposed may be referred to as the “cis side”, whereas the side at which second flow cell 120a-n is disposed may be referred to as the “trans side”. In some cases, samples may be loaded from both the cis side and the trans side, the designation of which may now be arbitrary. For the purposes of this disclosure, a “reference” is a composition of known identity, concentration, and physical/chemical properties to which a sample may be compared to result in one or more measurements.

With continued reference to FIG. 1, first flow cell 120a-n intersects second flow cell 120a-n at a junction 124. For the purposes of this disclosure, a “junction” is a point of contact that joins first flow cell 120a-n, second flow cell 120a-n, and nanopore 104 together, such that one or more species may pass through the nanopore 104, with various extents of selectivity, from one flow cell 120a-n to the other, in either direction. Such species may include without limitation ions, molecules, microbes, or the like. At least a nanopore 104, as described above, is located at junction 124 and connecting between first flow cell 120a-n and second flow cell 120a-n. Each of first flow cell 120a-n and second flow cell 120a-n may contain at least an opening along its pathlength, wherein the at least an opening of first flow cell 120a-n may face the at least an opening of second flow cell 120a-n and be separated by nanopore 104. As a nonlimiting example, sample may be prepared in a buffer solution, such as a phosphate-buffered saline (PBS) solution, whereas reference may be a blank phosphate-buffered saline (PBS) solution.

With continued reference to FIG. 1, for the purposes of this disclosure, a “buffer” or “buffer solution” is a solution or mixture that contains at least a pair of weak acid, HA, and its conjugate base, A, (i.e., the weak acid minus one proton) in a molar ratio between 10:1 and 1:10, wherein the solution maintains a stable pH close to the pKₐ (i.e., the negative log of the acid dissociation constant, Kₐ) of the weak acid, against addition of acidic or basic chemical species. For simplicity, a buffer containing a pair of conjugate base and acid may be written as A/HA. Additional examples will be provided below. The pH of a buffer solution may be calculated using the Henderson Hasselbalch equation:

$$p H = p {K}_{a} + log ( \frac{[ {A}^{-} ]}{[ H A ]} )$$

With continued reference to FIG. 1, a buffer may include any type of buffer deemed suitable by a person of ordinary skill in the art upon reviewing the entirety of this disclosure. As another nonlimiting example, a buffer may include an acetate buffer (i.e., CH₃COONa/CH₃COOH). As another nonlimiting example, a buffer may include a borate buffer (i.e., Na₂B₄O₇·10H₂O/H₃BO₃). As another nonlimiting example, a buffer may include a bicarbonate buffer (i.e., NaHCO₃/H₂CO₃ or Na₂CO₃/NaHCO₃, depending on the desired pH). As another nonlimiting example, a buffer may include a cacodylate buffer (i.e., NaC₂H₆AsO₂/HC₂H₆AsO₂). As another nonlimiting example, a buffer may include a Good's buffer. For the purposes of this disclosure, “Good's buffers” are a group of more than 20 conjugate acid/base pairs selected and described by Norman Good and colleagues for biochemical and biological research during 1966-1980. For simplicity, only the conjugate acid may be shown for each conjugate acid/base pair. Good's buffers include MES (C₆H₁₃NO₄S), ACES (C₄H₉NO₄S), PIPES (C₈H₁₈N₂O₆S₂), MOPS (C₇H₁₅NO₄S), TES (C₆HaNO₆S), HEPES (C₈H₁₈N₂O₄S), Tricine (C₆H₁₃NO₅), TRIS (C₄H₁₁NO₃), Bicine (C₆H₁₃NO₄), TAPS (C₇H₁₇NO₆S), CHES (C₈H₁₇NO₃S), CAPS (C₉H₁₉NO₃S), AMPSO (C₉H₁₉NO₄S), Gly-Gly (C₄H₁₁N₂O₃), ADA (C₄H₇NO₄), BES (C₆H₁₅NO₅S), MOPSO (C₇H₁₅NO₅S), EPPS (C₉H₂ON₂O₄S), HEPPS (C₁₁H₂₄N₂O₄S), CAPSO (CH₁₉NO₄S), HEPPSO (C₉H₂ON₂O₅S), CABS (C₁₀H₁₉NO₃S), ACESO (C₄H₉NO₅S), TES-Na (C₆H₁₄NO₆SNa), BICINE-Na (C₆H₁₂NO₄Na), TRICINE-Na (C₆H₁₂NOSNa), MES-Na (C₆H₁₂NO₄SNa), HEPES-Na (C₈H₁₇N₂O₄SNa), MOPS-Na (C₇H₁₄NO₄SNa), and PIPES-Na (C₈H₁₇N₂O₆S₂Na). As a nonlimiting example, buffer may include a phosphate buffer (i.e., NaH₂PO₄/H₃PO₄, Na₂HPO₄/NaH₂PO₄, or Na₃HPO₄/Na₂HPO₄, depending on the desired pH). As another nonlimiting example, buffer may include a phosphate-buffered saline (PBS) solution, a commonly used buffer in biological research and pharmaceutical formulations that typically contains 137 mM NaCl, 2.7 mM KCl, 10 mM Na₂HPO₄, and 1.8 mM KH₂PO₄. A person of ordinary skill in the art, upon reviewing the entirety of this disclosure, will be able to recognize suitable buffers for apparatus 100.

With continued reference to FIG. 1, in one or more embodiments, apparatus 100 may include a growth medium in which a microbe may grow and replicate. Nonlimiting examples of suitable growth media for bacteria may include general-purpose nutrient broths, Luria-Bertani (LB) broth, Tryptic Soy Broth (TSB), Brain Heart Infusion (BHI) Broth, Mueller-Hinton Broth, Thioglycollate Broth, Alkaline Peptone Water (APW), Selenite F Broth, Buffered Peptone Water, MR-VP Broth, and/or the like. Nonlimiting examples of suitable growth media for fungi may include Sabouraud Dextrose Broth (SDB), Potato Dextrose Broth (PDB), Malt Extract Broth (MEB), Brain Heart Infusion (BHI) Broth, Czapek-Dox Broth, Yeast Extract Peptone Dextrose (YPD) Broth, Inhibitory Mold Broth (IMB), Mycosel/Mycobiotic Broth, Cornmeal Broth, Niger Seed Broth, and/or the like. In some cases, a growth medium may be mixed with blood for certain fastidious bacteria that are hard to culture. In some cases, a growth medium may be kept at a high CO₂ partial pressure and/or in an environment that lacks oxygen, for facultative anaerobic or strictly anaerobic bacteria.

With continued reference to FIG. 1, apparatus 100 may be used to generate a microbial growth curve. Specifically, to generate a bacterial/fungal growth curve, bacteria or fungi may first be grown in a culture broth, as described above. Aliquots from such culture broth may be taken (e.g., at certain time delays) and analyzed using apparatus 100. Alternatively, aliquots from such culture broth may be centrifuged, using a centrifuge tube, to form a pellet. The supernatant of this centrifuged mixture may be discarded subsequently, and the remaining pellet may be resuspended using a buffer, such as PBS, as described above in this disclosure. The resuspended pellet may then be analyzed using apparatus 100. To generate a viral growth curve, a different approach may be taken: first, a suitable eukaryotic cell line may be cultivated and infected using a virus; such cell culture may optionally be monitored for an appearance of cytopathic effects, which may signify that an infection has successfully occurred. The supernatant of this eukaryotic culture medium may then be sampled as aliquots and analyzed by apparatus 100.

With continued reference to FIG. 1, apparatus 100 comprises at least a detector 128 connected to plurality of flow cells 120a-n, wherein the at least a detector 128 is configured to detect a signal as a function of at least a translocated microbe from sample. For the purposes of this disclosure, a “signal” is any intelligible representation of data. A signal may be transmitted from one device to another. A signal may include an optical signal, a hydraulic signal, a pneumatic signal, a mechanical signal, an electric signal, a digital signal, an analog signal, and the like. In some cases, a signal may be used to communicate with a computing device, for example by way of one or more ports. In some cases, a signal may be transmitted and/or received by a computing device, for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), Universal Serial Bus (USB) connection, SPI (Serial Peripheral Interface) protocol, I2C (Inter-Integrated Circuit) protocol, Bluetooth communication, Wireless Fidelity (WIFI) communication, and the like. “Signal”, “trace”, and “signal trace” may be used interchangeably throughout this disclosure. In one or more embodiments, signal may be an electrical signal, which may include and/or be generated by a change to any electrical parameter that results from an event or object to be detected. Exemplary types of electrical signal include, without limitation, electrical current, voltage difference (e.g., bias or zeta potential), impedance, capacitance, inductance, or the like. In one or more embodiments, signal may be an optical signal within at least a characteristic wavelength, frequency, and amplitude within the electromagnetic spectrum, such as absorbance, optical density, light scattering, fluorescence, phosphorescence, rotational and/or vibrational signatures, or any similar applicable spectroscopic features recognized by a person of ordinary skill in the art upon reviewing the entirety of this disclosure. A signal may contain one or more events to be identified. For the purposes of this disclosure, an “event” is an occurrence of change within a spatial and/or temporal trace of data (i.e., a signal) that deviates from a stable baseline value beyond a certain detection threshold and potentially contains useful information related to one or more functions of apparatus 100, as described below in this disclosure. For example and without limitation, such detection threshold may be expressed in terms of an absolute physical quantity (e.g. a current of at least 10 nA), a relative physical quantity (e.g., 1% with respect to the baseline), a certain number of standard deviations to account for noise (e.g., 5 standard deviations of noise at baseline), or a combination thereof using the max( ) or min( ) operators and/or one or more logical operators. In one or more embodiments, signal may rise and decay as a function of time and/or space to result in at least an event with a shape of a peak or pulse, with one or more attributes embedded therein, as described below.

With continued reference to FIG. 1, for the purposes of this disclosure, a “detector” is a device configured to capture at least a signal and/or one or more events contained therein, as described below. In one or more embodiments, detector 128 may be an electrical detector that detects one or more changes in electrical signal due to translocation of microbe (e.g., as microbe enters or leaves nanopore 104). Detector 128 may include an ammeter, a voltmeter, and/or one or more variations thereof. In one or more embodiments, detector 128 may include a resistive pulse sensor, such as a tunable resistive pulse sensor. In such embodiments, a signal may be detected by detector 128 as a resistive pulse (i.e., a spike of increased electrical resistance) due to a displacement/exclusion of conductive species from nanopore 104 as one or more microbes are translocated therethrough. In one or more embodiments, detector 128 may include an optical sensor such as a photodetector, an optical absorption spectrometer, an optical emission spectrometer, and/or the like. For the purposes of this disclosure, an “optical sensor” is a device that detects one or more changes in optical signal. An optical sensor may detect a change in optical signal due to translocation of microbe (e.g., as microbe enters or leaves nanopore 104). For the purposes of this disclosure, a “photodetector” is a device or component that, upon receiving at least a photon, generates a measurable change in at least an electrical parameter within a circuit incorporating the photodetector. As a result, other components of a circuit may amplify, detect, record, or otherwise use such signal for purposes that include without limitation analysis of the detected at least a photon, which may be combined with analyses of photons detected by other photodetectors, imaging based on detected photons, and other similar purposes. Photodetectors are a subclass of optical sensors. Photodetector may include, without limitation, PIN diodes, avalanche photodiodes (APDs), single photon avalanche diodes (SPADs), silicon photomultipliers (SiPMs), photo-multiplier tubes (PMTs), micro-channel plates (MCPs), micro-channel plate photomultiplier tubes (MCP-PMTs), indium gallium arsenide semiconductors (InGaAs), photodiodes, phototransistors, and/or photosensitive or photon-detecting circuit elements, semiconductors and/or transducers. For the purposes of this disclosure, avalanche photo diodes (APDs) are diodes (e.g. without limitation p-n, p-i-n, and others) reverse-biased such that a single photo-generated carrier can trigger a short, temporary “avalanche” of photocurrent on the order of milliamps or more caused by electrons being accelerated through a high field region of the diode and impact-ionizing covalent bonds in the bulk material, these in turn triggering greater impact ionization of electron-hole pairs. APDs provide a built-in stage of gain through avalanche multiplication. When the reverse bias is less than the breakdown voltage, the gain of the APD is approximately linear. For silicon APDs, this gain is on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse-biased significantly above the breakdown voltage is referred to as a single photon avalanche diode, or SPAD. In this case, the n-p electric field is sufficiently high to sustain an avalanche of current with a single photon, hence referred to as “Geiger mode”. This avalanche current rises rapidly (on a sub-nanosecond timescale), such that detection of the avalanche current can be used to approximate the arrival time of the incident photon. The SPAD may be pulled below breakdown voltage once triggered in order to reset or quench the avalanche current before another photon may be detected, as while the avalanche current is active, carriers from additional photons may have a negligible effect on the current in the diode.

With continued reference to FIG. 1, a plurality of photodetectors may be in close proximity to each other. For instance, each photodetector may be placed directly next to neighboring photodetectors of plurality of photodetectors, for instance in a two-dimensional grid, a grid on a curved surface or manifold, or the like. Placement in close proximity may eliminate or reduce to a negligible level spatially dependent variation in received signals, permitting a control circuit, as described below, to infer other causes for signal variation between detectors. As a nonlimiting example, an array of photodetectors may be comprised of photodetectors occupying a length or breadth of less than 25 μm, permitting a resolution of more than 1,600 per square millimeter; by introducing electrical connections on a second level of a multilevel wafer, or similar techniques, the resolution of the array may be limited only by the package size and/or fabrication size of photodetectors.

With continued reference to FIG. 1, photodetectors and/or array of photodetectors may be constructed using any suitable fabrication method. Fabrication may be performed by assembling one or more electrical components and/or photodetectors in one or more circuits. Electrical components may include passive and active components, including without limitation resistors, capacitors, inductors, switches or relays, voltage sources, and the like. Electrical components may include one or more semiconductor components, such as diodes, transistors, and the like, consisting of one or more semiconductor materials, such as without limitation silicon, germanium, indium, gallium, arsenide, nitride, mercury, cadmium, and/or telluride, processed with dopants, oxidization, and ohmic connection to conducting elements such as metal leads. Some components may be fabricated separately and/or acquired as separate units and then combined with each other or with other portions of circuits to form circuits. Fabrication may depend on the nature of a component; for instance, and without limitation, fabrication of resistors may include forming a portion of a material having a known resistivity in a length and cross-sectional volume producing a desired degree of resistance, an inductor may be formed by performing a prescribed number of wire winding about a core, a capacitor may be formed by sandwiching a dielectric material between two conducting plates, and the like. Fabrication of semiconductors may follow essentially the same general process in separate and integrated components as set forth in further detail below; indeed, individual semiconductors may be grown and formed in lots using integrated circuit construction methodologies for doping, oxidization, and the like, and then cut into separate components afterwards. Fabrication of semiconductor elements, including without limitation diodes, transistors, and the like, may be achieved by performing a series of oxidization, doping, ohmic connection, material deposition, and other steps to create desired characteristics; persons skilled in the art, upon reviewing the entirety of this disclosure, will be aware of various techniques that may be applied to manufacture a given semiconductor component or device.

With continued reference to FIG. 1, one or more components and/or circuits may be fabricated together to form an integrated circuit. This may generally be achieved by growing at least a wafer of semiconductor material, doping regions of it to form, for instance, npn junctions, pnp junctions, p, n, p+, and or n+ regions, and/or other regions with local material properties, to produce components and terminals of semiconductor components such as base, gate, source and drain regions of a field-effect transistor such as a so-called metal oxide field-effect transistor (MOSFET), base, collector and emitter regions of bipolar junction BJT transistors, and the like. Common field-effect transistors include but are not limited to carbon nanotube field-effect transistor (CNFET), junction gate field-effect transistor (JFET), metal-semiconductor field-effect transistor (MESFET), high-electron-mobility transistor (HEMT), metal-oxide-semiconductor field-effect transistor (MOSFET), inverted-T field-effect transistor (ITFET), fin field-effect transistor (FinFET), fast-recovery epitaxial diode field-effect transistor (FREDFET), thin-film transistor, organic field-effect transistor (OFET), ballistic transistor, floating-gate transistor, ion-sensitive field-effect transistor (IFSET), electrolyte-oxide-semiconductor field-effect transistor (EOSFET), and/or deoxyribonucleic acid field-effect transistor (DNAFET). A person of ordinary skill in the art will be aware of various forms or categories of semiconductor devices that may be created, at least in part, by introducing dopants to various portions of a wafer. Further fabrication steps may include oxidization or other processes to create insulating layers, including without limitation at the gate of a field-effect transistor, formation of conductive channels between components, and the like. In one or more embodiments, logical components may be fabricated using combinations of transistors and the like, for instance by following a complimentary MOSFET (CMOS) process whereby desired element outputs based on element inputs are achieved using complementary circuits each achieving the desired output using active-high and active-low MOSFETS or the like. CMOS and other processes may similarly be used to produce analog components and/or components or circuits combining analog and digital circuit elements. Deposition of doping material, etching, oxidization, and similar steps may be performed by selective addition and/or removal of material using automated manufacturing devices in which a series of fabrication steps are directed at particular locations on the wafer and using particular tools or materials to perform each step; such automated steps may be directed by or derived from simulated circuits as described in further detail below.

With continued reference to FIG. 1, fabrication may include the deposition of multiple layers of wafer; as a nonlimiting example, two or more layers of wafer may be constructed according to a circuit plan or simulation which may contemplate one or more conducting connections between layers; circuits so planned may have any three-dimensional configuration, including overlapping or interlocking circuit portions, as described in further detail below. Wafers may be bound together using any suitable process, including adhesion or other processes that securely bind layers together; in some embodiments, layers are bound with sufficient firmness to make it impractical or impossible to separate layers without destroying circuits deposited thereon. Layers may be connected using vertical interconnect accesses (VIA or via), which may include, as a nonlimiting example, holes drilled from a conducting channel on a first wafer to a conducting channel on a second wafer and coated with a conducting material such as tungsten or the like, so that a conducting path is formed from the channel on the first wafer to the channel on the second wafer. VIAs may also be used to connect one or more semiconductor layers to one or more conductive backing connections, such as one or more layers of conducting material etched to form desired conductive paths between components, separate from one another by insulating layers, and connected to one another and to conductive paths in wafer layers using VIAs.

With continued reference to FIG. 1, each photodetector of plurality of photodetectors may have at least a signal detection parameter. As used herein, a signal detection parameter is a parameter controlling the ability of a photodetector to detect at least a photon and/or one or more properties of a detected photon. In one or more embodiments, a signal detection parameter may determine what characteristic or characteristics at least a photon directed to the photodetector must possess to be detected. For instance, a signal detection parameter may include a wavelength and/or frequency at which a photon may be detected, a time window within which detection is possible at a particular photodetector, an angle of incidence, polarization, or other attributes or factors as described in further detail below. A signal detection parameter may include an intensity level of the at least a photon, i.e. a number of photons required to elicit a change in at least an electrical parameter in a circuit incorporating the at least a photodetector. Plurality of photodetectors may have heterogenous signal detection parameters; signal detectors and/or signal detection parameters may be heterogeneous where the plurality of photodetectors includes at least a first photodetector having a first signal detection parameter of the at least a signal detection parameter and at least a second photodetector having a second signal detection parameter of the at least a signal detection parameter, and where the at least a first signal detection parameter differs from the at least a second signal detection parameter. Heterogenous signal detection parameters may assist array in eliminating noise, increase the ability of array to detect attributes of tissue being sampled, and/or increase the temporal resolution of array.

With continued reference to FIG. 1, at least a signal detection parameter may include a temporal detection window. For the purposes of this disclosure, a temporal detection window is a period of time during which a photodetector is receptive to detection of photons, such as when an SPAD is in pre-avalanche mode as described above. Temporal detection window may be set by a delay after a given event or time, including reception of signal by another photodetector. This may be accomplished using delay circuitry. Delay circuitry may operate to set photodetector to a receptive mode at the desired time. SPADs and other similar devices have the property that the bias voltage may be dynamically adjusted such that the detector is “off” or largely insensitive to incoming photons when below breakdown voltage, and “on” or sensitive to incoming photons when above breakdown voltage. Once a current has been registered indicating photon arrival, the diode may be required to be reset via an active or passive quenching circuit. This may lead to a so-called “dead time” in which no arriving photons are counted. Varied temporal detection windows may permit a control circuit as described below to set bias voltages in a sequence corresponding to initiation of each temporal detection window, so that while one detector is quiescent, other nearby detectors are capable of receiving signals. As a nonlimiting example, a first signal detection parameter may include a first temporal detection window, a second signal detection parameter may include a second temporal detection window, and at least a portion of the first temporal detection window may not overlap with the second temporal detection window.

With continued reference to FIG. 1, delay circuitry may also block circuit transmission of signals from photodetectors that are outside their temporal detection windows, for instance by passing output of photodetectors through a Boolean “AND” gate having a second input at delay circuitry and passing a “false” value to the second input for any detector outside its temporal detection window. The increase in temporal and/or spatial resolution of a SPAD or other photodetector may have several advantages when applied to 2D or 3D imaging of biological tissue, such as the eye or other organ, based on a time-of-flight measurement device or the like. This may particularly be the case when interested in detecting time-varying signals with good spatial resolution. In a representative use, time-varying absorption of photons may be correlated to blood oxygenation. In another use, Doppler flow measurement may be more accurate in a system with greater time and/or spatial resolution. This approach may have additional utility in industrial applications e.g. automotive Lidar, where the ability to increase spatial and/or temporal resolution within all or some regions of the field of view is of interest.

With continued reference to FIG. 1, setting of receptive modes of photodetectors and/or intensity levels at which photodetectors emit detection signals may be controlled using a bias control circuit. Bias control circuit may function to set a bias of a photodetector to enable detection of some quantity of photons. In the case of SPAD detector, voltage bias of diode may be programmable in one or more steps such that the SPAD may be reverse-biased above the breakdown voltage of the junction in order to enable “Geiger-mode” single photon detection or biased below breakdown voltage to enable linear gain detection mode. In the case of other detector types of variable gain (e.g. PMT, MCP, MCP-MPT, photodiode, or the like), voltage bias may be programmable to enable adjustable gain. Gain may be fixed, adjusted dynamically via feedback from the incident photon flux (e.g. to avoid saturation), or via other means, e.g. lookup table or other. In an embodiment, gain may be used to determine an intensity of a detected at least a photon. Voltage bias control of the detector may be triggered via some means, such as without limitation via local delay elements such as buffer circuits, fixed or programmable or triggered by a timing reference, e.g., a reference clock edge or the like. In the case of SPAD detector, detector bias control may incorporate an active, passive or combination quenching circuit to reset the diode. Reset signal may be based on photocurrent reaching a threshold level, change in photocurrent level (e.g. via sense amplifier) or other. Detector bias control may incorporate stepwise voltage level adjustment to minimize after-pulsing and other noise sources. Detector bias control may incorporate adiabatic methods to recover energy and reduce power of a high voltage bias system. System may incorporate delay logic, which may include, without limitation, local delay elements fixed or programmable and/or controlled via other reference timing circuitry. Delay logic may incorporate feedback from the incident photon flux or via other means, such as without limitation a lookup table or other. A person of ordinary skill in the art, upon reviewing the entirety of this disclosure, will be able to identify how to select and/or implement one or more photodetectors for apparatus 100.

With continued reference to FIG. 1, in one or more embodiments, detector 128 may include a reactive sensor. For the purposes of this disclosure, a “reactive sensor” is a sensor that is configured to detect an approaching microbe in its proximity and apply a probing mechanism in response to the approaching microbe. As a nonlimiting example, where a bacterium approaches nanopore 104, a reactive sensor may anticipate one or more events to occur, as a signal deviates from its baseline beyond a certain threshold, and quickly apply a bias in response, wherein apparatus 100 may subsequently determine how the bacterium reacts to the applied bias. For instance, detector 128 may instantly change the voltage when it detects an occurrence of an event, where a reaction of the bacterium may be measured and recorded while the bacterium is within a proximity of nanopore 104. Such reactive sensor may highlight/exacerbate some electrical properties of a particle, such as a microbe, that may otherwise not be readily resolved by a standard apparatus. For example, and without limitation, a reactive sensor may resolve a particle based on its charge, which may otherwise not be readily resolved when a liquid flow is dominated by a hydrostatic pressure. As another nonlimiting example, a reactive sensor may resolve a particle based on its electric polarizability.

With continued reference to FIG. 1, apparatus 100 includes a control unit 132 communicatively connected to at least a detector 128. For the purposes of this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device. Communicative connecting may also include indirect connections via, for example and without limitation, wireless connection, radio communication, low-power wide-area network, optical communication, magnetic, capacitive, or optical coupling, and the like. In some instances, the terminology “communicatively coupled” may be used in place of communicatively connected in this disclosure.

With continued reference to FIG. 1, in one or more embodiments, control unit 132 may include a computing device. Computing device could include any analog or digital control circuit, including an operational amplifier circuit, a combinational logic circuit, a sequential logic circuit, an application-specific integrated circuit (ASIC), a field programmable gate arrays (FPGA), or the like. Computing device may include a processor communicatively connected to a memory, as described above. Computing device may include any computing device as described in this disclosure, including without limitation a microcontroller, microprocessor, digital signal processor, and/or system on a chip as described in this disclosure. Computing device may include, be included in, and/or communicate with a mobile device such as a mobile telephone, smartphone, or tablet. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a first computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a nonlimiting example, using a “shared nothing” architecture.

With continued reference to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. A person skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing. More details regarding computing devices will be described below.

With continued reference to FIG. 1, control unit 132 may include or be communicatively connected to a database or a datastore of similar nature. For the purposes of this disclosure, a “database” is an organized collection of data or a type of data store based on the use of a database management system (DBMS), the software that interacts with end users, applications, and the database itself to capture and analyze the data. Database may be implemented, without limitation, as a relational database, a key-value retrieval database such as a NoSQL database, or any other format or structure for use as database that a person of ordinary skill in the art would recognize as suitable upon review of the entirety of this disclosure. Database may alternatively, or additionally, be implemented using a distributed data storage protocol and/or data structure, such as a distributed hash table or the like. Database may include a plurality of data entries and/or records as described in this disclosure. Data entries in database may be flagged with or linked to one or more additional elements of information, which may be reflected in data entry cells and/or in linked tables such as tables related by one or more indices in database or another relational database. A person of ordinary skill in the art, upon reviewing the entirety of this disclosure, will be aware of various ways in which data entries in database may store, retrieve, organize, and/or reflect data and/or records as used herein, as well as categories and/or populations of data consistently with this disclosure.

With continued reference to FIG. 1, in some cases, control unit 132 may be configured to query a database by searching within the database for a match. As a nonlimiting example, when a database includes a SQL database, control unit 132 may be configured to submit one or more SQL queries to interact with the database. To retrieve data, a “SELECT” statement may be used to specify one or more columns, rows, table names, and/or the like, and optional conditions may be applied using WHERE clauses. In some cases, a DBMS may use indexes, if available, to quickly locate relevant rows and columns, ensuring accurate and efficient data retrieval. Once SQL queries are executed using a DBMS interface or code, results may be returned for further steps.

With continued reference to FIG. 1, computing device may perform determinations, classification, and/or analysis steps, methods, processes, or the like as described in this disclosure using machine-learning processes. For the purposes of this disclosure, a “machine-learning process” is a process that automatedly uses a body of data known as “training data” and/or a “training set” to generate an algorithm that will be performed by a processor module to produce outputs given data provided as inputs. This is in contrast to a nonmachine-learning software program where the commands to be executed are determined in advance by a user and written in a programming language. A machine-learning process may utilize supervised, unsupervised, self-supervised, and/or lazy-learning processes, and/or neural network architectures. More details regarding computing devices and machine-learning processes will be provided below.

With continued reference to FIG. 1, in one or more embodiments, control unit 132 may be configured to perform one or more of its functions using a machine-learning model, as described below. In one or more embodiments, computing device may include a machine-learning module to implement one or more algorithms or create one or more machine-learning models to generate outputs. However, machine-learning module is exemplary and may not be necessary to create one or more machine-learning models and perform any machine learning described herein. In one or more embodiments, one or more machine-learning models may be generated using training data. Training data may include inputs and corresponding predetermined outputs so that machine-learning module may use correlations between the provided exemplary inputs and outputs to develop an algorithm and/or relationship that then allows a machine-learning model to determine its own outputs for inputs. Training data may contain correlations that a machine-learning process may use to model relationships between two or more categories of data elements. Exemplary inputs and outputs may come from measurements collected using standard solutions of microbe with known identities, computer simulations, user inputs, or the like, as described below. In one or more embodiments, machine-learning module may obtain training data by querying a communicatively connected database that includes past inputs and outputs. Training data may include inputs from various types of databases, resources, libraries, dependencies, and/or user inputs, as well as outputs correlated to each of those inputs, so that machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a nonlimiting example, training data may include data entered in standardized forms by persons or processes, such that entry of a given data element in a given field in a form may be mapped to one or more descriptors of categories. Elements in training data may be linked to categories by tags, tokens, or other data elements. Machine-learning module may be used to create at least a machine-learning model using training data. Training data may be data sets that have already been converted from raw data manually, by machine, or via any other method. In some cases, a machine-learning model may be trained based on user inputs. For example, a user may provide user feedback to indicate that information that has been output is inaccurate, wherein machine-learning model may be trained as a function of the user feedback. In some cases, a machine-learning model may allow for improvements to computing device, such as but not limited to an improvement relating to comparing data items, an ability to sort efficiently, an increase in accuracy of analytical methods, and the like.

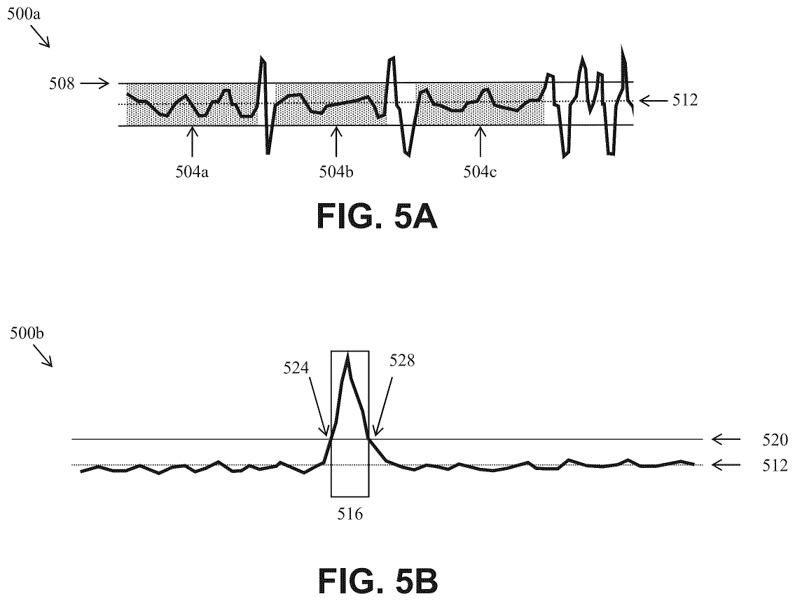

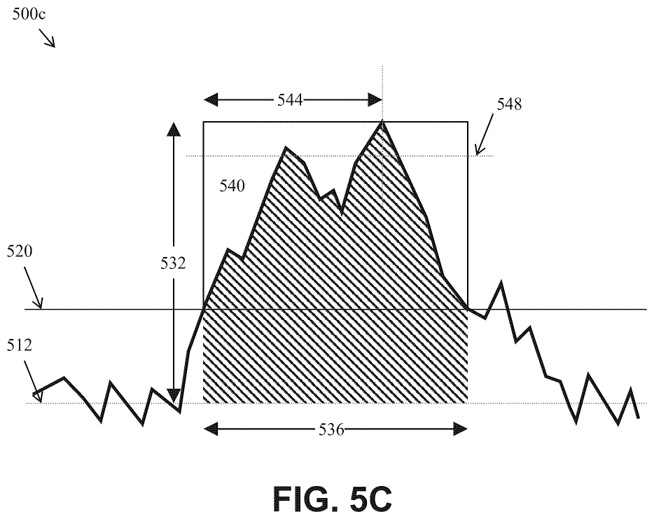

With continued reference to FIG. 1, control unit 132 is configured to correlate a first attribute and a second attribute of detected signal. For the purposes of this disclosure, an “attribute” of a signal is a quantitative feature describing one or more aspects of the signal. Nonlimiting examples of such attributes may include peak height, peak area, peak shape, peak symmetry, peak linewidth, peak multiplicity, among others. Additional details pertaining to such attributes may be provided below in this disclosure when discussing FIGS. 5A-C. In one or more embodiments, correlating first attribute and second attribute of detected signal may include receiving correlation training data including a plurality of exemplary correlations as outputs correlated to a plurality of exemplary signal attributes as inputs. Accordingly, in one or more embodiments, correlating first attribute and second attribute of detected signal may further include iteratively training a correlation machine-learning model using correlation training data. Accordingly, in one or more embodiments, correlating first attribute and second attribute of detected signal may further include correlating the first attribute and the second attribute of the detected signal using trained correlation machine-learning model. For the purposes of this disclosure, an “exemplary signal attribute” is a standard feature of a signal detected or simulated based on a microbe of known identity or a variant closely related thereto, capturing one or more prominent characteristics to expected from measuring a sample containing the microbe. In one or more embodiments, an exemplary signal attribute may be embedded in one or more exemplary events, as described in detail below in this disclosure. In one or more embodiments, correlation training data may include one or more exemplary signal attributes collected using solutions of pure microbes with known identities. In one or more embodiments, correlation training data may include one or more simulated signals or events synthesized using a computer software such as COMSOL. In one or more embodiments, correlation training data may include data specifically synthesized for training purposes using one or more generative models. In one or more embodiments, one or historical measurements (e.g., historical signals and events therein) may be incorporated into correlation training data upon validation. In one or more embodiments, correlation training data may be retrieved from one or more databases and/or other repositories of similar nature or be supplied as one or more inputs from a user. In one or more embodiments, at least a portion of correlation training data may be added, deleted, replaced, or otherwise updated, either automatically according to present metrics or as a function of one or more inputs from a user.

With continued reference to FIG. 1, a plurality of attributes may form a signal pattern. Likewise, a plurality of exemplary signal attributes may form one or more exemplary signal patterns. For the purposes of this disclosure, a “signal pattern” is an abstract representation of elements of portions within signal, reflecting one or more spatial relationships, attributes, or correlations therein, as described below. In one or more embodiments, signal patterns may be defined by a user via one or more user inputs, consistent with details described above. In one or more embodiments, signal patterns may be visualized in a coordinate system, e.g., as domains or clusters of data, to form a basis for identification of one or more microbes, as described in detail below in this disclosure.

With continued reference to FIG. 1, correlation machine-learning model may include or be implemented using any type of machine-learning model or algorithm described in this disclosure. In one or more embodiments, correlation machine-learning model may be a neural network such as without limitation a multi-layer perceptron (MLP), a one-dimensional convolutional neural network (1D-CNN), a recurrent neural network including long-short-term memory (LSTM), a transformer, a temporal convolutional network (TCN), or a deep neural network, decision trees, random forests, and XGBoost, among others, as described below. In one or more embodiments, correlation machine-learning model may implement one or more feature extraction and/or feature learning algorithms, as described below. For the purposes of this disclosure, multi-layer perceptron (MLP) is a class of feedforward artificial neural network, which includes multiple layers of nodes or neurons arranged in a layered structure. MLP is one of the foundational architectures in neural networks and is commonly used for tasks like classification, regression, and pattern recognition. Additional details pertaining to MLP will be provided below in this disclosure. For the purposes of this disclosure, a “one-dimensional convolutional neural network (1D-CNN)” is a type of convolutional neural network (CNN) specifically designed to process and analyze one-dimensional sequential data. Unlike traditional neural networks, which operate on individual data points, 1D CNNs apply convolutional filters along one dimension of the data, making them particularly well-suited for tasks involving time series, signals, and other sequential data. Additional details pertaining to CNNs and 1D-CNNs will be provided below in this disclosure. For the purposes of this disclosure, a “recurrent neural network (RNN)” is a type of neural network designed to handle sequential data. RNNs may capture temporal dependencies, but sometimes may struggle with long sequences due to the vanishing gradient problem. Additional details pertaining to RNNs will be provided below in this disclosure. For the purposes of this disclosure, “long-short-term memory (LSTM)” is a specialized recurrent neural network designed to handle long-term dependencies in sequential data. LSTM may use memory cells and gates to control the flow of information, making it suitable for tasks involving sequences such as natural language processing, speech recognition, and time series analysis. By addressing the shortcomings of traditional recurrent neural networks, LSTMs may be highly effective for capturing both short-term and long-term patterns in complex sequences. Additional details pertaining to LSTM and variations thereof will be provided below in this disclosure. For the purposes of this disclosure, a “temporal convolutional network (TCN)” is a type of convolutional neural network designed specifically for sequence data. TCNs may use dilated convolutions to capture information over larger spans of the sequence. For the purposes of this disclosure, “XGBoost” or “Extreme Gradient Boosting” is a highly efficient and scalable machine learning algorithm that belongs to the family of gradient boosting algorithms. XGBoost is designed to improve the performance of decision tree-based models by combining multiple weak learners (typically decision trees) to create a strong learner. XGBoost is widely used in data science and machine learning competitions because of its speed, accuracy, and ability to handle large, complex datasets. Additional details pertaining to XGBoost will be provided below in this disclosure.

With continued reference to FIG. 1, in one or more embodiments, correlating first attribute and second attribute may include correlating the first attribute and the second attribute using principal component analysis (PCA), as described below. For the purposes of this disclosure, “principal component analysis” is a statistical technique used to simplify complex datasets by reducing their dimensionality while preserving as much of the data's variability as possible. PCA achieves this goal by identifying new axes, i.e., principal components. For the purposes of this disclosure, a “principal component” is a new variable in PCA that represents a direction in a data space along which data varies the most. Each principal component is a linear combination of original variables in a dataset and captures the maximum possible variance in the data along that direction. Principal components may be ordered according to the amount of variance they capture. In other words, a first principal component may capture the maximum variance in the data, whereas a second principal component may capture the next highest variance, and so on. Principal components are also orthogonal and uncorrelated to each other. As a result, an original dataset may be represented with fewer dimensions, typically two or three, and therefore simplified, while retaining the most significant information.

With continued reference to FIG. 1, a PCA process typically begins with standardizing the dataset, especially if variables are measured on different scales. After this standardization step, a covariance matrix may be computed to understand how these variables are correlated with each other. For the purposes of this disclosure, a “covariance matrix” is a square matrix that represents pairwise covariances between variables in a dataset. For a dataset with multiple variables, a covariance matrix may capture how each pair of variables relate to (i.e., changes with) one another. Specifically, each element in a covariance matrix shows a covariance between two variables. A positive covariance may indicate that a first variable tends to increase as a second variable increases (i.e., a positive correlation), whereas a negative covariance may indicate that a first variable tends to decrease as a second variable increases (e.g., a negative correlation). Diagonal elements of a covariance matrix represent variances of each individual variable.

With continued reference to FIG. 1, a PCA process includes calculating eigenvalues and eigenvectors a covariance matrix. For the purposes of this disclosure, an “eigenvalue” of PCA is an amount of variance captured by a principal component. The eigenvalues are scalars that indicate the magnitude of variance along each principal component, with larger eigenvalues corresponding to principal components that capture more of the data's variance. Eigenvalues may help prioritize which principal components are the most important, with the first principal component having the highest eigenvalue and therefore capturing the largest variance. For the purposes of this disclosure, an “eigenvector” is the direction of a principal component in a multi-dimensional space of original variables. Each eigenvector includes a vector of coefficients that define a linear combination of original variables. The direction of an eigenvector may indicate how much each original variable contributes to a principal component. In other words, eigenvectors provide axes or directions along which data can be projected to capture the maximum variance. In PCA, eigenvectors corresponding to the largest eigenvalues form the basis for a new coordinate system in which data is expressed, with each principal component aligned along these eigenvectors. A person of ordinary skill in the art, upon reviewing the entirety of this disclosure, will be able to recognize suitable means of implementing PCA in apparatus 100.

With continued reference to FIG. 1, in one or more embodiments, correlating first attribute and second attribute may include correlating the first attribute and the second attribute using linear discriminant analysis (LDA), as described below. For the purposes of this disclosure, “linear Discriminant Analysis” is another statistical technique used primarily for dimensionality reduction and classification tasks. LDA seeks to find a linear combination of features that best separates two or more classes of objects or events. The main goal of LDA may be to maximize the separation between the classes by finding the projection that increases the distance between the means of the classes while minimizing the variance within each class.

With continued reference to FIG. 1, a LDA process typically begins by calculating the mean vectors for each class in a dataset, followed by a computation of within-class and between-class scatter matrices. A within-class scatter matrix may capture the spread of data points within each class, while a between-class scatter matrix may capture the spread of the mean vectors between multiple classes. LDA then determines the eigenvalues and eigenvectors of the matrix, consistent with details described above pertaining to PCA, that results from a ratio between a between-class scatter to a within-class scatter. Eigenvectors corresponding to the largest eigenvalues may be used to construct linear discriminants, which are the directions that maximize class separation.

With continued reference to FIG. 1, PCA and LDA are both powerful techniques for dimensionality reduction that may be used for one or more data analysis steps in apparatus 100. While they share a common goal of reducing the number of features in a dataset to make analysis more manageable and to enhance the performance of machine-learning models, they differ fundamentally in their objectives, methodologies, and applications. PCA is an unsupervised learning technique primarily focused on identifying the directions, or principal components, along which the variance in the data is maximized. PCA does not take into account any class labels associated with the data; it simply aims to reduce the dimensionality by projecting the data onto a lower-dimensional subspace that retains most of the original variability. PCA is particularly useful when the goal is to compress the data while preserving the intrinsic structure, making it easier to visualize or perform subsequent analysis. In contrast, LDA is a supervised learning technique that takes class labels into account. The primary objective of LDA is to maximize the separation between different classes in the data by finding the linear combinations of features that best discriminate between the classes. LDA achieves this by maximizing the ratio of the between-class variance to the within-class variance, ensuring that the classes are as distinct as possible when projected onto the new feature space. Unlike PCA, which focuses solely on the variance within the data, LDA seeks to preserve the class separability, making it particularly useful for classification tasks. While PCA may be applied to any dataset without the need for labeled data, LDA requires labeled data and is thus inherently linked to classification problems. PCA may often be used as a preprocessing step to reduce the dimensionality of data before applying other machine-learning algorithms, including LDA. On the other hand, LDA may be directly used for dimensionality reduction. In terms of computational complexity, PCA is generally less computationally intensive than LDA, especially when the number of classes is large. This is because LDA involves computing scatter matrices and solving an eigenvalue problem with respect to these matrices, which becomes more complex as the number of classes increases. Additional details regarding PCA and LDA will be provided below in this disclosure.

With continued reference to FIG. 1, control unit 132 is further configured to identify one or more types of microbes as a function of correlation. Such identification may be performed based on one or more types of signal patterns, consistent with details described in U.S. patent application Ser. No. 18/663,425 (attorney docket number 1624-001USU1), filed on May 14, 2024, entitled “APPARATUS AND METHODS FOR IDENTIFICATION OF MICROBIAL PRESENCE”, the entirety of which is incorporated herein by reference. In some cases, a first type of microbe may have a first type of correlation between a first and a second attribute of its detected signal, and a second type of microbe may have a second type of correlation between the first and the second attribute of its detected signal, wherein the second type of correlation is different from the first type of correlation. In some cases, a correlation may be visualized graphically, and accordingly, control unit 132 may identify a microbe based on such correlation, such as by performing a classification task based on a cluster or domain in which the correlation is located.

With continued reference to FIG. 1, in some cases, identification of one or more types of microbes may include implementing an identification machine-learning model. Specifically, control unit 132 may receive identification training data including a plurality of exemplary identification results as outputs correlated to a plurality of exemplary correlations as inputs. Control unit 132 may then train identification machine-learning model as a function of identification generation training data and identify one or more microbes using the identification machine-learning model. Implementation of such identification machine-learning model may be consistent with any type of machine-learning model or algorithm described in this disclosure. In one or more embodiments, identification training data may include data specifically synthesized for training purposes using one or more generative models. In one or more embodiments, one or historical measurements may be obtained from samples for which a “ground truth” is known. For the purposes of this disclosure, a “ground truth” is a property that a machine-learning model is configured to predict. For the invention described herein, such ground truth may include the identity of one or more microbial species and/or one or more specific external stimuli or conditions applied thereto. Exemplary stimuli may include an antibiotic drug, to which resistant bacteria may adapt by changing their morphology. Such a change in morphology may result in one or more changes in the attributes of a detected event. Exemplary conditions may include without limitation osmolarity. A different osmolarity may change how turgid a bacterial cell may be. Hypoosmolarity makes bacteria swell, while hyperosmolarity makes them shrink. These changes may also result in one or more changes in the attributes of a detected event. These ground truths may be utilized to train a machine-learning model on a classification task to identify the presence/absence of one or more microbial species and/or one or more specific external stimuli or conditions applied thereto in future measurements. For the purposes of disclosure, “training” is a process of updating the internal coefficients, weights, and/or parameters within a machine-learning model to ensure it can perform its intended function or functions in a reproducible manner. Such function may include without limitation classifying events according to the species associated therewith and/or the stimuli/conditions applied thereto. For the purposes of this disclosure, “reproducible”, as opposed to overfitting, is an indication that a machine-learning model performs satisfactorily not only on training data, but also on new data that have not been input before. In one or more embodiments, online learning may be utilized for such purposes after a machine-learning model has completed its initial training. Specifically, new examples with a known ground truth may be shown to a machine-learning model, and the weights therein may be adjusted, effectively resuming training. This technique may be useful for the invention described herein, as microbes may change (e.g., mutate) over time. For example, and without limitation, every year, one or more new Influenzavirus strains may become dominant among the general population. In this case, it may be beneficial to update such machine-learning model on at least a yearly basis to ensure it is applicable to the new variant or variants. In one or more embodiments, identification training data may be retrieved from one or more databases and/or other repositories of similar nature or be supplied as one or more inputs from a user. In one or more embodiments, at least a portion of identification training data may be added, deleted, replaced, or otherwise updated as a function of one or more inputs from a user. A person of ordinary skill in the art, upon reviewing the entirety of this disclosure, will be able to recognize suitable means to implement identification machine-learning model in apparatus 100.

With continued reference to FIG. 1, control unit 132 is further configured to classify a plurality of events within detected signal based on identified one or more types of microbes. This classification function is missing in widely used Coulter counters and is useful for describing the effect of a certain chemical on a specific sub-population of microbes. For antibiograms, a clinical sample for an infectious disease usually does not contain only the pathogen; it likely contains the possible pathogen alongside numerous other bacterial families which make up the microbiota (e.g., oral, intestinal, and respiratory, among others). In other words, for every use case of antibiotic, there may be some bacteria that are naturally resistant to it and accordingly form a “natural background”. For example, beta-lactams are most efficient in killing Gram-positive bacterial, while Gram-negative bacteria are often partially or completely insensitive to them (with some exceptions including Escherichia coli, Klebisella, Haemophilus, Neisseria gonorrhoeae, and Proteus mirabilis, among others). As a result, one cannot simply apply a Coulter counter on a respiratory sample; instead, to check if a pathogen is sensitive to an antibiotic, the pathogen needs to be identified from other sub-populations of natural microbiota. Similarly, when evaluating the efficacy of preservatives added to food and cosmetic products, such products may contain a background with a variety of other particles that may share the same size with the bacteria of interest. These particles may include other ‘wanted’ bacteria/yeast, microplastics, and/or small aggregates of the complex matrix for a product, among others. To evaluate if a preservative is effectively preventing certain bacteria from growing, one may not simply count particles; instead, a particle needs to be deemed a bacterium of interest before being counted towards the concentration of the bacteria of interest.

With continued reference to FIG. 1, in one or more embodiments, classifying plurality of events may include classifying the plurality of events using a binary classification algorithm. For the purposes of this disclosure, a “binary classification algorithm” is an algorithm that classifies an element into one of two classes. As a nonlimiting example, a binary classification algorithm may be implemented to classify a plurality of events as either “events contributed by pathogens” or “events contributed by non-pathogens”. As another nonlimiting example, a binary classification algorithm may be implemented to classify a plurality of events as either “events of interest” or “events not of interest”. Control unit 132 may then quantify one or more microbes based on an outcome of such classification step, as described in detail below. Utilizing a binary classification approach, thereby restricting the classes to just two, may boost the quantification accuracy of apparatus 100. In other words, a machine-learning model may be more effective if it only has to quantify one type of bacteria vs all other species instead of quantifying each individual species. Similarly, in one or more embodiments, classifying plurality of events may include classifying the plurality of events into more than two classes, using a multi-class classifier (MCC) or a multi-class classification algorithm. As a nonlimiting example, such multi-class classifier (MCC) or a multi-class classification algorithm may classify plurality of events into a plurality of classes or bins, based on the identity of each detected microbe and/or one or more stimuli/conditions applied thereto.

With continued reference to FIG. 1, in one or more embodiments, classifying plurality of events may include classifying the plurality of events using a classification machine-learning model or a classifier. Specifically, control unit 132 may be configured to receive classification training data including a plurality of exemplary classes as outputs correlated to a plurality of exemplary events as inputs, consistent with details described above. Accordingly, in one or more embodiments, classifying plurality of events may further include iteratively training classification machine-learning model using classification training data. Accordingly, in one or more embodiments, classifying plurality of events may further include classifying the plurality of events using trained classification machine-learning model. In some cases, exemplary events may include events extracted from experimental data collected using one or more purified microbial samples, consistent with details described above. Accordingly, in some cases, exemplary signal attributes, as described above, may include exemplary signal attributes extracted from such exemplary events. Implementation of such classification machine-learning model may be consistent with any type of machine-learning model or algorithm described in this disclosure. In one or more embodiments, classification training data may include data specifically synthesized for training purposes using one or more generative models. In one or more embodiments, one or historical measurements may be incorporated into classification training data upon validation and used to retrain classification machine-learning model, such as by selecting or adjusting one or more weights therein. In one or more embodiments, classification training data may be retrieved from one or more databases and/or other repositories of similar nature or be supplied as one or more inputs from a user. In one or more embodiments, at least a portion of classification training data may be added, deleted, replaced, or otherwise updated as a function of one or more inputs from a user. A person of ordinary skill in the art, upon reviewing the entirety of this disclosure, will be able to recognize suitable means to implement classification machine-learning model in apparatus 100.

With continued reference to FIG. 1, control unit 132 is further configured to quantify at least one type of microbe of identified one or more types of microbes as a function of classified plurality of events. In some cases, such quantification may be based on a peak height or peak area of one or more events. In some cases, such quantification may be based on the number of events detected per unit of time. In some cases, such quantification may be reported as a concentration, number density, number of microbe aggregates (i.e., individual particles made of one or more microorganisms) per unit volume, or the like, such as the number of microbes in thousands (k) or millions (M) per unit volume such as per milliliter (mL), deciliter (dL), or liter (L), among others. In some cases, such quantification may be reported as a time-dependent function, such as a growth curve. Additional details will be provided below in this disclosure.

With continued reference to FIG. 1, control unit 132 may be configured to perform feature extraction on one or more identified events. For the purposes of this disclosure, “feature extraction” is a process of transforming an initial data set into informative measures and values. For example, feature extraction may include a process of determining one or more geometric features of detected signal. In one or more embodiments, feature extraction may be used to determine one or more spatial relationships, attributes, or correlations within signal/peak that may be used to identify one or more microbes that contributed to the signal/peak. In one or more embodiments, control unit 132 may be configured to extract one or more regions of interest, wherein the regions of interest may be used to extract one or more features using one or more feature extraction techniques.

With continued reference to FIG. 1, control unit 132 may be configured to perform one or more of its functions, such as feature extraction, using a feature learning algorithm. For the purposes of this disclosure, a “feature learning algorithm” is a machine-learning algorithm that identifies associations between elements of data in a data set where particular outputs and/or inputs are not specified. Data set may include without limitation a training data set. For instance, and without limitation, a feature learning algorithm may detect co-occurrences of elements of data, as defined above, with each other. Computing device may perform feature learning algorithm by dividing elements or sets of data into various sub-combinations of such data to create new elements of data and evaluate which elements of data tend to co-occur with which other elements. In one or more embodiments, feature learning algorithms may perform clustering of data.

With continued reference to FIG. 1, in one or more embodiments, a machine-learning model, such as correlation machine-learning model, identification machine-learning model, classification machine-learning model, quantification machine-learning model, and/or the like, may be iteratively trained as a function of user feedback. In one or more embodiments, a user may provide feedback to a machine-learning model, such as feedback indicating incorrect identification of one or more attributes. In one or more embodiments, a machine-learning model may be iteratively updated and/or re-trained, wherein user may provide feedback following each iteration of the processing. In one or more embodiments, iteratively training a machine-learning model may allow for faster processing, optimization of computer efficiency, and the like.

With continued reference to FIG. 1, in one or more embodiments, control unit 132 may include or be configured to communicate with at least a display device while performing one or more of its functions. Accordingly, control unit 132 may be configured to display one or more results via a user interface, such as a graphical user interface. In one or more embodiments, control unit 132 may be configured to communicate with a display device to directly display at least a detected signal. In one or more embodiments, control unit 132 may be configured to communicate with a display device to display one or more attributes and/or one or more correlations as a function of a detected signal. In one or more embodiments, control unit 132 may be configured to communicate with a display device to display at least an identity of a microbe as a function of a detected signal. Additional details regarding how signal is processed and how attributes therein are extracted are provided below in this disclosure. In one or more embodiments, control unit 132 may be configured to communicate with display device to display at least a result of classification/quantification for one or more detected microbes. For the purposes of this disclosure, a “display device” is a computer device that is either part of control unit 132 or a secondary device separate and distinct from but communicatively connected to control unit 132. A display device may include a desktop, a laptop, a smartphone, a tablet, or the like. In one or more embodiments, a display device may be communicatively connected to control unit 132 such as, for example, through network communication, through Bluetooth communication, and/or the like. In one or more embodiments, a user may submit one or more user inputs through a user interface, such as a graphical user interface, displayed using a display device.

With continued reference to FIG. 1, for the purposes of this disclosure, a “user interface” is a means by which a user and a computer system interact, for example, through the use of input devices and software. User interface may include graphical user interface (GUI), command line interface (CLI), menu-driven user interface, touch user interface, voice user interface (VUI), form-based user interface, any combination thereof, and the like. In one or more embodiments, a user may interact with user interface using computing device distinct from and communicatively connected to control unit 132, such as a desktop, a laptop, a smartphone, a tablet, or the like operated by the user. A user interface may include one or more graphical locator and/or cursor facilities allowing user to interact with graphical models and/or combinations thereof, for instance using a touchscreen, touchpad, mouse, keyboard, and/or other manual data entry device. For the purposes of this disclosure, a “graphical user interface” is a type of user interface that allows end users to interact with electronic devices through visual representations. In one or more embodiments, a graphical user interface may include icons, menus, other visual indicators or representations (graphics), audio indicators such as primary notation, display information, and related user controls. A menu may contain a list of choices and may allow users to select one from them. A menu bar may be displayed horizontally across the screen as a pull-down menu. A menu may include a context menu that appears only when user performs a specific action. Files, programs, web pages, and the like may be represented using a small picture within graphical user interface.

With continued reference to FIG. 1, in one or more embodiments, a graphical user interface may contain one or more interactive elements. For the purposes of this disclosure, an “interactive element” is an element within graphical user interface that allows for communication with control unit 132 by a user. For example, and without limitation, interactive elements may include a plurality of tabs wherein selection of a particular tab, such as for example, by using a fingertip, may indicate to a system to perform a particular function and display the result through graphical user interface. In one or more embodiments, an interactive element may include tabs within a graphical user interface, wherein the selection of a particular tab may result in a particular function. In one or more embodiments, interactive elements may include words, phrases, illustrations, and the like to indicate a particular process that user would like system to perform. A person of ordinary skill in the art, upon reviewing the entirety of this disclosure, will be aware of various ways in which user interfaces, graphical user interfaces, and/or elements thereof may be implemented and/or used as described in this disclosure.

With continued reference to FIG. 1, in one or more embodiments, a display device and/or remote device may be configured to display at least an event handler graphic corresponding to at least an event handler. For the purposes of this disclosure, an “event handler graphic” is a graphical element with which user interacts using a display device and/or remote device to enter data, such as without limitation commands, annotations, or the like. Event handler graphic may include, without limitation, a button, a link, a checkbox, a text entry box and/or window, a drop-down list, a slider, or any other event handler graphic deemed suitable by a person of ordinary skill in the art upon reviewing the entirety of this disclosure. For the purposes of this disclosure, an “event handler” is a module, data structure, function, and/or routine that performs an action on display device and/or remote device in response to one or more user inputs. For instance, and without limitation, an event handler may record data corresponding to user selections of previously populated fields such as drop-down lists and/or text auto-complete and/or default entries, data corresponding to user selections of checkboxes, radio buttons, or the like, potentially along with automatically entered data triggered by such selections, user entry of textual data using a keyboard, touchscreen, speech-to-text program, or the like. An event handler may generate prompts for further information, may compare data to validation rules such as requirements that the data in question be entered within certain numerical ranges, and/or may modify data and/or generate warnings to user in response to such requirements. An event handler may convert data into expected and/or desired formats, for instance such as date formats, currency entry formats, name formats, or the like. An event handler may transmit data from a remote device to apparatus 100, control unit 132, and/or computing device.

With continued reference to FIG. 1, in one or more embodiments, an event handler may include a cross-session state variable. For the purposes of this disclosure, a “cross-session state variable” is a variable recording data entered on remote device during a previous session. Such data may include, for instance, previously entered text, previous selections of one or more elements as described above, or the like. For instance, cross-session state variable data may represent a search that user entered in a past session. Cross-session state variable may be saved using any suitable combination of client-side data storage on a remote device and server-side data storage on a computing device; for instance, data may be saved wholly or in part as a “cookie” which may include data or an identification of remote device to prompt provision of cross-session state variable by the computing device, which may store the data on the computing device. Alternatively, or additionally, computing device may use login credentials, device identifier, and/or device fingerprint data to retrieve cross-session state variable, which the computing device may transmit to remote device. Cross-session state variable may include at least a prior session datum. A prior session datum may include any element of data that may be stored in cross-session state variable. An event handler graphic may be further configured to display at least a prior session datum, for instance and without limitation, by auto-populating user query data from previous sessions.

With continued reference to FIG. 1, in one or more embodiments, control unit 132 and/or computing device may configure display device and/or remote device to generate a graphical view. For the purposes of this disclosure, a “graphical view” is a data structure that results in display of one or more graphical elements on a screen. A graphical view may include at least a display element. For the purposes of this disclosure, a “display element” is an image that a program and/or data structure cause to be displayed. Display elements may include, without limitation, windows, pop-up boxes, web browser pages, display layers, and/or any other display element deemed relevant by a person of ordinary skill in the art upon reviewing the entirety of this disclosure. A graphical view may include at least a selectable event graphic corresponding to one or more selectable event handlers. For the purposes of this disclosure, a “selectable event graphic” is a graphical element that, upon selection, will trigger an action to be performed. Selection may be performed using a cursor or other locator as manipulated using a locator device such as a mouse, touchscreen, track pad, joystick, or the like. As a nonlimiting example, a selectable event graphic may include a redirection link. For the purposes of this disclosure, a redirection link is a hyperlink, button, image, portion of an image, and/or other graphic containing or referring to a uniform resource locator (URL) and/or other resource locator to another graphical view including without limitation buttons, and/or to a process that performs navigation to such URL and/or other resource locator upon selection of a selectable event graphic. Redirection may be performed using any event handler, including without limitation event handlers detecting the click of a mouse or other locator, access of redirection link using a touchscreen, the selection of any key, mouseover events, or the like.

With continued reference to FIG. 1, in one or more embodiments, apparatus 100 may include a Faraday cage 136 that is connected to control unit 132 and encapsulates the rest of apparatus 100. For the purposes of this disclosure, a “Faraday cage” or “Faraday shield” is an enclosure used to shield what it encloses from external electric fields; in other words, apparatus 100 is electrically grounded, via either a cable or direct contact. A Faraday shield may be formed by a continuous covering of a conductive material such as a metal, or in the case of a Faraday cage, by a mesh of such materials. Using Faraday cage 136 may ensure that apparatus 100 (and control unit 132 contained therein) performs one or more of its functions described herein without being disturbed by external electromagnetic radiation.

Referring now to FIGS. 2A-C, exemplary embodiments 200a-c of nanopores 104 and various parameters related thereto are illustrated. FIG. 2A includes an exemplary cross-sectional view 200a of nanopore 104 within a thin-layer matrix 108. In one or more embodiments, nanopore 104 may be described by a geometry, as described below. For the purposes of this disclosure, a “geometry” of nanopore 104 is a three-dimensional (3D) representation of a contour of nanopore 104 in both its longitudinal and lateral directions, capturing all features (such as projections, recesses, or the like) in every angle and direction; it may be any feasible and/or applicable geometry for construction of nanopore 104 and/or use of apparatus 100, such as right or oblique circular cylinder, elliptic cylinder, right rectangular prism, right square prism, triangular prism, pentagonal prism, hexagonal prism, parallelepiped, rhombohedron, trigonal trapezohedron, truncated sphere, truncated ellipsoid, and/or the like. In one or more embodiments, nanopore 104 may be described by two additional parameters: a longitudinal dimension (i.e., thickness) 204 and a lateral dimension 208 (i.e., size), as described above. For the purposes of this disclosure, lateral dimension 208 of nanopore 104 is the longest lateral distance from one side of an orifice to another, through a straight line. As a nonlimiting example, for nanopore 104 with a right circular cylinder geometry, lateral dimension 208 is the diameter of the circular cross section. As another nonlimiting example, for nanopore 104 with an elliptic cylinder geometry, lateral dimension 208 is the length of the major axis of the elliptical cross section. As another nonlimiting example, for nanopore 104 with a right rectangular prism geometry, lateral dimension 208 is the length of the diagonal of the rectangular cross section.

With continued reference to FIG. 2A, nanopore 104 may have an aspect ratio, as shown in FIG. 2A. For the purposes of this disclosure, an “aspect ratio” is a ratio between longitudinal dimension 204 and lateral dimension 208. In one or more embodiments, aspect ratio may have an impact on the selectivity of nanopore 104 and/or apparatus 100 towards microbe 212. It is proposed that a lower aspect ratio may contribute to a greater ability of nanopore 104 to differentiate morphological and biochemical subtle features of microbe 212. A small longitudinal dimension 204 (e.g., a thin-layer matrix 108 with a smaller thickness) may be beneficial for achieving superior resolution for apparatus 100, although a longitudinal dimension 204 that is too small may result in excessive frailty of nanopore 104. Additional details will be provided below in this disclosure.

With continued reference to FIG. 2A, a microbe 212 may be translocated through nanopore 104. For the purposes of this disclosure, a “translocation” of microbe 212 refers to the transport of the microbe 212 through nanopore 104, entering from one open end and leaving from the other. Translocation of microbe 212 may be a result of electrophoresis, diffusion, hydrostatic pressure, a combination thereof, or the like. A person of ordinary skill in the art, upon reviewing the entirety of this disclosure, may recognize the large number of parameters involved for selective detection of microbe 212 and the strategies for selecting them, as described below. In one or more embodiments, translocation of microbe 212 may result in a temporary displacement of ionic species within nanopore 104, which may likely cause a reduction of conductivity and results in a resistive pulse, as described below. It is worth noting that translocation of microbe 212 through nanopore 104 may not be the only mechanism to result in detection of events. As a nonlimiting example, microbe 212 (e.g., a bacterium) or a particle similar thereto may move via Brownian motion, approach nanopore 104 and its orifice, and change its direction of motion. Such trajectory may still leave a footprint in a signal, although the signal is likely nuanced compared to a full translocation event.

With continued reference to FIGS. 2A-C, in some cases, in order to impart sufficient mechanical strength to nanopore 104, the nanopore 104 may be excavated from a conical cavity within thin-layer matrix 108 that tapers from one side of thin-layer matrix 108 to the other side of thin-layer matrix 108, as shown in FIGS. 2B-C, embodiments 200b and 200c. In some cases, the surface of such conical cavity may form an angle with a longitudinal axis of nanopore 104. This angle may also be referred to as an aperture angle. As a nonlimiting example, this angle may be between 20° and 70°, such as 45°, as shown in FIG. 2C. Such conical design may result in a smaller opening on one end of thin-layer matrix 108, with a dimension of d, and a larger opening on the other side of thin-layer matrix 108, with a dimension of D. In some cases, such as for a nanopore 104 with a cylindrical shape, d may be the same as lateral dimension 208. The thickness of think-layer matrix 108 may be represented by H, whereas longitudinal dimension 204 may be represented by h. Similarly, as another nonlimiting example, nanopore 104 may be excavated from a cylindrical cavity within thin-layer matrix 108 (not shown).

With continued reference to FIGS. 2A-C, in one or more embodiments, nanopore 104 may be excavated in a membrane of organic materials, such as without limitation polyimides (PIs) and/or polyurethane (PUs or PURs). As a nonlimiting example, nanopore 104 may be excavated in a membrane of polyimide with a 12.5-micrometer longitudinal dimension 204 and a 4-micrometer lateral dimension 208.

With continued reference to FIGS. 2A-C, in one or more embodiments, as shown in FIG. 2B, nanopore 104 may be excavated in a SiNₓ wafer. For the purposes of this disclosure, “SiNₓ” or “silicon nitride” is a type of ceramic material containing one or more compounds formed by silicon (Si) and nitrogen (N); the ratio between Si and N in SiNₓ is typically 3:4 but may vary from case to case. As a nonlimiting example, nanopore 104 may be excavated in a 4 millimeter×4 millimeter wafer of fused silica with a 200-micrometer thickness D. As another nonlimiting example, nanopore 104 may be constructed with a longitudinal dimension 204 of 50 nanometers. As another nonlimiting example, nanopore 104 may be excavated with a 300-nanometer, 500-nanometer, 2-micrometer, or 4-micrometer lateral dimension 208 (d).

With continued reference to FIGS. 2A-C, in one or more embodiments, as shown in FIG. 2C, nanopore 104 may be excavated in a glass wafer, such as without limitation a fused-silica wafer or a silicon oxide wafer. As a nonlimiting example, nanopore 104 may be excavated in a 4 millimeter×4 millimeter wafer of fused silica with a 160-micrometer thickness D. As another nonlimiting example, nanopore 104 may be constructed with a longitudinal dimension 204 between 10 micrometers and 15 micrometers. As another nonlimiting example, nanopore 104 may be excavated with a 2-micrometer or 4-micrometer lateral dimension 208 (d).

With continued reference to FIGS. 2A-C, the type of material used to construct nanopore 104 may impose various manufacturing constraints such as a minimum thickness of thin-layer matrix 108 and/or a minimum longitudinal dimension 204 of nanopore 104. As a nonlimiting example, a nanopore 104 constructed using SiNₓ may have a minimum longitudinal dimension 204 on the order of 50 nanometers, whereas a nanopore 104 constructed using glass may have a minimum longitudinal dimension 204 between 10 micrometers and 15 micrometers. Due to the relatively large diameter of bacteria, a larger lateral dimension 208 (e.g., >2 micrometers) may be chosen for nanopore 104; accordingly, under a comparable aspect ratio, longitudinal dimension 204 of nanopore 104 may also increase, thereby relaxing the manufacturing constraint of the nanopore 104.

With continued reference to FIGS. 2A-C, nanopore 104 may be constructed using a graphene layer, a molybdenum disulfide (MoS₂) layer, a gallium arsenide (GaAs) wafer, an indium gallium arsenide (InGaAs) wafer, an indium phosphide (InP) wafer, a silicon carbide (SiC) wafer, a diamond-like carbon (DLC) wafer, an aluminum oxide (Al₂O₃) wafer, a titanium nitride (TiN) wafer, a titanium dioxide (TiO₂) wafer, a hafnium oxide (HfO₂), a zirconium oxide (ZrO₂) wafer, a boron nitride (BN) wafer, or a ceramic wafer, among others, as recognized by a person of ordinary skill in the art, upon reviewing the entirety of this disclosure.

With continued reference to FIGS. 2A-C, in one or more embodiments, nanopore 104 may include a coating layer, as shown in FIG. 2C. In some cases, nanopore 104 may be coated/lined with a secondary material, such as aluminum oxide (AlOₓ) or silicon oxide (SiOₓ), either across an extended surface area of thin-layer matrix 108 or only locally at the pore, to fine-tune its chemical properties such as surface charges. In some cases, the elemental ratio within coating material or materials may be fine-tuned to achieve certain enrichment or deficiency of one or more elements, and such variations may create new properties that are not present in their stoichiometric analogues. As a nonlimiting example, in aluminum oxide, the ratio between Al³⁺ and O²⁻ may be synthetically tuned to deviate from 1:3. In some cases, such as without limitation for optical measurements, gold and/or other metals may be used as a coating layer. In some cases, for optical and electrical measurements performed in parallel, metals coated with an insulator coating layer on one side may be used.

With continued reference to FIGS. 2A-C, in one or more embodiments, a coating layer may include an organic coating layer. In some cases, such organic coating layer may include a self-assembled monolayer. For the purposes of this disclosure, a “self-assembled monolayer” is a single layer of organic molecules anchored to a surface that spontaneously organize themselves into a well-ordered structure. Organic molecules may form a self-assembled monolayer due to intermolecular interactions such as van der Waals interactions and/or London dispersion forces, and such formation is driven by the second law of thermodynamics to minimize the Gibbs free energy of an exposed surface. Organic molecules that form self-assembled monolayers typically contain a head group with a binding affinity to a surface, a tail group that extends away from the surface (which may be either polar or nonpolar, depending on the exact use case), and a spacer in between. The exact structure of a resulting self-assembled monolayer may also depend on factors such as the length, geometry, and/or aromaticity of the organic molecules, their packing density (which may depend on the type of surface used), and lattice constant matching/mismatching, among others. As a nonlimiting example, a densely packed self-assembled monolayer on a planar surface may have a highly ordered, crystalline structure, whereas a loosely packed self-assembled monolayer on a curved surface may contain more defects such as gaps or grain boundaries. As another nonlimiting example, longer, linear organic molecules may form a more ordered, crystalline self-assembled monolayer than short, branched organic molecules. Organic molecules in a self-assembled monolayer may include a ligand. For the purposes of this disclosure, a “ligand” is a chemical species capable of binding with and stabilizing another chemical species, often a metal or metal ion, through coordinate covalent bond. A ligand may include a neutral molecule or an ion, and usually contains relatively polarizable elements such as O, N, P, S, or the like with lone-pair electrons available for forming coordinate covalent bonds. A ligand may include a surfactant with a hydrophilic head portion and a hydrophobic tail portion. A ligand may include any type of ligand deemed suitable by a person of ordinary skill in the art, upon reviewing the entirety of this disclosure, such as without limitation alkoxysilanes, amines, thiols/thiolates, phosphonic acids/phosphonates, carboxylic acids/carboxylates, alcohols, sulfonic acids/sulfonates, among others.

With continued reference to FIGS. 2A-C, as a nonlimiting example, a self-assembled monolayer may include a fluorinated self-assembled monolayer and/or otherwise contain a fluorinated portion; in other words, an organic molecule/ligand within a self-assembled monolayer may include one or more fluorine atoms. Due to the large electronegativity (4.0) and low polarizability of fluorine atoms, fluorinated organic molecules often have weak intermolecular forces compared to their nonfluorinated analogues. As a result, a fluorinated self-assembled monolayer often exhibits antifouling properties that prevents undesired binding adhesion of ions, molecules, or cells, in a manner similar to the Teflon coating of a nonstick pan. As a nonlimiting example, fluorinated self-assembled monolayers may be used to prevent the formation of biofilms and/or the clogging of nanopore 104.

With continued reference to FIGS. 2A-C, in some cases, a coating layer may be applied to nanopore 104 using techniques such as epitaxial growth, spin coating, dip coating, solution deposition, spray coating, vapor deposition including chemical vapor deposition (CVD) and physical vapor deposition (PVD), electroplating, electroless plating, powder coating, thermal spraying, Langmuir-Blodgett deposition, roll-to-roll coating, slot die coating, inkjet printing, electrospinning, sol-gel coating, vapor phase polymerization, or the like, as recognized by a person of ordinary skill in the art upon reviewing the entirety of this disclosure. In some cases, preparation of a coating layer may include rinsing and drying steps to remove impurities. In some cases, a coating layer may be annealed, cured, or sintered to enhance its mechanical properties and prevent peeling.

With continued reference to FIGS. 2A-C, in some cases, nanopore 104 may be recycled by rinsing the nanopore 104 with an anticlogging agent, such as a surfactant solution including without limitation Triton X and/or a nonpolar solvent such as without limitation isoprene. Certain microbes may form aggregates with one another, likely via hydrophobic contacts, and block nanopore 104. By applying a an anticlogging agent to nanopore 104, these aggregates may be disintegrated and removed, and as a result, nanopore 104 may be reused for additional measurements. In some cases, clogging of nanopore 104 may be minimized or prevented by adding an anticlogging agent into a sample and/or a reference. In some cases, clogging of nanopore 104 may be minimized or prevented by increasing or reducing the concentration of electrolytes (and maximizing or minimizing the screening effect thereof). Additionally, and/or alternatively, applying pulses of counter-pressure that creates a net flow of liquid from the trans-side to the cis-side of nanopore 104 or applying transversal convective currents to rinse the front and back sides of the nanopore 104 may also be beneficial in at least partially mitigating and/or resolving clogging issues associated therewith.

Referring now to FIGS. 3A-D, exemplary embodiments 300a-d of various designs of flow cells nanopore readers are illustrated. Related elements such as nanopore 104, electrodes 112a-b, flow cell 120a-n, microbe 212, are also included, consistent with details described above in this disclosure. FIG. 3A illustrates a basic design with two chambers and a wafer of thin-layer matrix 108 in between, wherein nanopore 104 is located within the wafer. FIG. 3B illustrates a design with flow cells 120a-n containing two conical cavities and nanopore 104 sandwiched in between, consistent with details described above pertaining to FIGS. 2B-C. FIG. 3B illustrates a design with flow cells 120a-n of irregular shapes and nanopore 104 disposed in between. FIG. 3B illustrates a design with flow cells 120a-n connecting to form a U-shaped tube and nanopore 104 disposed in between. Flow cells 120a-n and/or nanopore readers 116 of such designs or the like may be manufactured using any suitable means as recognized by a person of ordinary skill in the art, upon reviewing the entirety of this disclosure, such as without limitation 3D printing and injection molding. It is worth noting that the design of flow cells 120a-n and/or nanopore reader 116 may not limited to embodiments described in this disclosure, and the choice of design may be dependent on types of materials and exact use cases, among other constraints.

Referring now to FIG. 4A-B, a plurality of nanopores 104 that are different in one way or another may be disposed in various configurations to achieve pore multiplicity, and accordingly, identification and/or quantification of microbe 212 may depend on such multiplicity, consistent with details described above. In some cases, plurality of nanopores 104 may be arranged in a line. In some cases, plurality of nanopores 104 may be arranged in a two-dimensional (2D) array or matrix. In some cases, plurality of nanopores 104 may be arranged in a three-dimensional (3D) array or matrix. In some cases, plurality of nanopores 104 may be dispersed over a plurality of locations, wherein each location of the plurality of locations contains either a single nanopore 104 or a cluster of multiple nanopores 104. Additional details will be described below in this disclosure.

With continued reference to FIGS. 4A-B, A), a plurality of configurations may exist for integrating plurality of nanopores 104 within nanopore reader 116. In one or more embodiments, a set of n nanopores 104 may be disposed between n independent first flow cells 120a-n and n independent second flow cells 120a-n. Specifically, one nanopore 104 may be disposed between each pair of first flow cell 120a-n and second flow cell 120a-n, with one electrode connected to each first flow cell 120a-n and each second flow cell 120a-n. In one or more embodiments, two or more nanopores 104 may share one first flow cell 120a-n and/or one second flow cell 120a-n. In one or more embodiments, a set of n nanopores 104 may be disposed between a shared first flow cell 120a-n and n independent second flow cells 120a-n. In one or more embodiments, a set of n nanopores 104 may be disposed between and n independent first flow cells 120a-n a shared second flow cell 120a-n.

Referring now to FIG. 4A, three exemplary illustrations 400a1-3 are shown for plurality of nanopores 104 arranged in a line, such as a straight line, a curved line, or a loop. Plurality of nanopores 104 may have two or more different configurations to achieve selective translocation of one or more microbes 212. In one or more embodiments wherein plurality of nanopores 104 is arranged in a line, as shown in 400a1, at least a first nanopore 104 within plurality of nanopores 104 may have a first voltage difference 404a along a first longitudinal axis of the at least a first nanopore 104, at least a second nanopore 104 within the plurality of nanopores 104 may have a second voltage difference 404b along a second longitudinal axis of the at least a second nanopore, and the first voltage difference 404a is different from the second voltage difference 404b. In one or more embodiments, as shown in 400a1, each nanopore 104 within plurality of nanopores 104 may have a unique voltage difference (404a-n, i.e., 404a, 404b, 404c, 404d, . . . , as shown in different shadings), as described above. In addition, plurality of nanopores 104 may differ by a number of other factors. In some nonlimiting examples, they may differ based on different pressures (such as without limitation a pressure differential between 0 and ±50 mmH₂O), different pre-processing filtering mechanisms, wavelength used for detection, pore materials, types of coating, and the like.

With continued reference to FIG. 4A, in one or more embodiments wherein plurality of nanopores 104 is arranged in a line, as shown in 400a2, at least a first nanopore 104 within plurality of nanopores 104 may have a first size 408a between 100 nanometers and 20 micrometers, at least a second nanopore 104 within the plurality of nanopores 104 may have a second size 408b between 100 nanometers and 20 micrometers, and the first size is different from the second size. In one or more embodiments, as shown in 400a2, each nanopore 104 within plurality of nanopores 104 may have a unique size (408a-n, i.e., 408a, 408b, 408c, 408d, . . . ) between 100 nanometers and 20 micrometers.

With continued reference to FIG. 4A, in one or more embodiments wherein plurality of nanopores 104 is arranged in a line, as shown in 400a3, at least a first nanopore 104 within plurality of nanopores 104 may have a first geometry 412a, at least a second nanopore 104 within the plurality of nanopores 104 may have a second geometry 412b, and the first geometry is different from the second geometry. In one or more embodiments, as shown in 400a3, each nanopore 104 within plurality of nanopores 104 may have a unique geometry (412a-n, i.e., 412a, 412b, 412c, 412d, . . . ).

Referring now to FIG. 4B, an exemplary embodiment 400b for plurality of nanopores 104 arranged in a 2D array/matrix is illustrated. For the purposes of this disclosure, “2D array” and “2D matrix” are both intended to represent one or more types of geometrical arrangement and/or topology among a plurality of elements within a substantially flat plane and may be used interchangeably. For the purposes of this disclosure, a “substantially flat” plane is a plane that is apparently flat to the naked eye. 2D matrix may be any 2D array/matrix, such as, without limitation, a square array, a rectangular array, an oblique array, a hexagonal array, or the like. A person of ordinary skill in the art, upon reviewing the entirety of this disclosure, will be recognize the various possible ways in which plurality of nanopores 104 may be arranged in 2D. In one or more embodiments, 2D matrix may include at least two axes: a first axis 416 and a second axis 420, wherein the first axis 416 extends in a direction that is different from the direction in which second axis 420 extends. In some cases, first axis 416 and second axis 420 may be perpendicular to each other. In some cases, first axis 416 and second axis 420 may be joined at an angle that is not 90°. In some cases, the assignment of first vs. second axis may be arbitrary.

With continued reference to FIG. 4B, in one or more embodiments wherein nanopores 104 are arranged in 2D matrix, plurality of nanopores 104 may be arranged along first axis 416, wherein at least a first nanopore 104 along the first axis 416 has a first size 408a between 100 nanometers and 20 micrometers, at least a second nanopore 104 along the first axis 416 has a second size between 100 nanometers and 20 micrometers, and the first size 408a is different from the second size 408b. Similarly, plurality of nanopores 104 may be arranged along second axis 420, wherein at least a first nanopore 104 along the second axis 420 has a first voltage difference 404a along a first longitudinal axis of the at least a first nanopore 104, at least a second nanopore 104 along the second axis 420 has a second voltage difference 404b along a second longitudinal axis of the at least a second nanopore 104, and the first voltage difference 404a is different from the second voltage difference 404b. In one or more embodiments wherein nanopores 104 are arranged in 2D matrix, plurality of nanopores 104 may be arranged along first axis 416, wherein each nanopore 104 within one or more nanopores 104 along the first axis 416 has a unique size between 100 nanometers and 20 micrometers that is different from the size of the rest of nanopores 104 along the first axis 416. Similarly, plurality of nanopores 104 may be arranged along second axis 420, wherein each nanopore 104 within one or more nanopores 104 along the second axis 420 has a unique voltage difference that's different from the voltage difference of the rest of nanopores 104 along the second axis 420.

With continued reference to FIG. 4B, in one or more embodiments wherein nanopores 104 are arranged in 2D matrix, at least a first nanopore 104 within plurality of nanopores 104 may have a first geometry, at least a second nanopore 104 within plurality of nanopores 104 may have a second geometry, and the first geometry is different from the second geometry, as described above for FIG. 4A. In one or more embodiments wherein nanopores 104 are arranged in 2D matrix, each nanopore 104 within one or more nanopores 104 along one or more axes (such as first axis 416, second axis 420, and/or the like) may have a geometry that's different from the geometry of the rest of nanopores 104 along that axis, as described above for FIG. 2B.

With continued reference to FIG. 4B, plurality of nanopores 104 may be arranged in 3D matrix. In some cases, 3D matrix may be dissected into a plurality of lines and/or a plurality of 2D matrices, consistent with details described above. In one or more embodiments wherein plurality of nanopores 104 is arranged in 3D matrix, at least a first nanopore 104 within the plurality of nanopores 104 may have a first size 408a between 100 nanometers and 20 micrometers, at least a second nanopore 104 within the plurality of nanopores 104 may have a second size 408b between 100 nanometers and 20 micrometers, and the first size is different from the second size. In one or more embodiments wherein plurality of nanopores 104 is arranged in 3D matrix, each nanopore 104 within the plurality of nanopores 104 may have a unique size (408a-n, i.e., 408a, 408b, 408c, 408d, . . . ) between 100 nanometers and 20 micrometers. In one or more embodiments wherein plurality of nanopores 104 is arranged in 3D matrix, at least a first nanopore 104 within the plurality of nanopores 104 may have a first voltage difference 404a along a first longitudinal axis of the at least a first nanopore 104, at least a second nanopore 104 within the plurality of nanopores 104 may have a second voltage difference 404b along a second longitudinal axis of the at least a second nanopore, and the first voltage difference 404a is different from the second voltage difference 404b. In one or more embodiments wherein plurality of nanopores 104 is arranged in 3D matrix, each nanopore 104 within the plurality of nanopores 104 may have a unique voltage difference (404a-n, i.e., 404a, 404b, 404c, 404d, . . . ). In one or more embodiments wherein plurality of nanopores 104 is arranged in 3D matrix, at least a first nanopore 104 within the plurality of nanopores 104 may have a first geometry, at least a second nanopore 104 within the plurality of nanopores 104 may have a second geometry, and the first geometry is different from the second geometry. In one or more embodiments, each nanopore 104 within one or more nanopores 104 may have a geometry that's different from the geometry of the rest of nanopores 104.

With continued reference to FIGS. 4A-B, it is worth noting that size 408a-n of nanopore 104, geometry 412a-n of nanopore 104, and voltage difference 404a-n applied to nanopore 104 are not the only tunable parameters applicable to apparatus 100. Instead, any tunable parameter that may resolve or differentiate one type of microbe 212 from another may be applied to apparatus 100, for example and without limitation, across one or more axes within a 2D array of nanopores 104. As a nonlimiting example, at least a first nanopore 104 may be applied with a first hydrostatic pressure, at least a second nanopore 104 may be applied with a second hydrostatic pressure, wherein the first hydrostatic pressure is different the second hydrostatic pressure. As another nonlimiting example, a pore surface of at least a first nanopore 104 of plurality of nanopores 104 may be made of a first material, and a pore surface of at least a second nanopore 104 of the plurality of nanopores 104 may be made of a second material, wherein the first material is different from the second material. As another nonlimiting example, a first flow cell 120a-n may contain a first buffer solution, a second flow cell 120a-n may contain a second buffer solution, wherein the first buffer solution is different from the second buffer solution. A person of ordinary skill in the art, upon reviewing the entirety of this disclosure, will be able to recognize additional parameters not disclosed in this application that may be applicable to apparatus 100.

Referring now to FIG. 5A, an exemplary embodiment of a signal 500a is illustrated. Analyzing signal 500a may include extracting one or more statistical characteristics from at least a portion of the signal trace, such as a mean (i.e., average), μ, or a standard deviation, σ. In one or more embodiments, detecting a signal or event comprises detecting at least a flat interval 504a-c. For the purposes of this disclosure, a flat interval is an isolated, well-defined spatial or temporal region within signal 500a wherein the first derivative of signal 500a does not deviate beyond a noise threshold 508. For the purposes of this disclosure, a noise threshold is an arbitrary cutoff that equals σ of the first derivative of entire signal 500a multiplied by a scaling factor. In one or more embodiments, noise threshold 508 may be equal to 1.5 times the σ of the first derivative of entire signal trace. In one or more embodiments, for detection of bacteria, flat interval 504a-c may have a minimum temporal duration of 0.1-0.3 milliseconds, a maximum duration of 2-4 milliseconds, and/or be separated from one another by at least an interval of 0.1 millisecond. In one or more embodiments, detecting at least a flat interval 504a-c may include processing signal 500a using a low-pass filter (e.g., a filter with a 1 kHz cutoff) first.

With continued reference to FIG. 5A, additionally and/or alternatively, one or more flat intervals 504a-c may be isolated, well-defined spatial or temporal regions within signal 500a wherein the signal 500a itself never leaves the expected baseline and stays within +a certain number of σ (e.g., ±2 g). For the purposes of this disclosure, an expected baseline is the mean or median of a signal in a longer time interval flanking an interval of interest. As a nonlimiting example, such longer time interval may be set as 10 s. As another nonlimiting example, flat intervals 504a-c may have a minimum duration of 1 s. It is worth noting that parameters described herein are exemplary values of hyperparameters that may be tuned according to specific use cases.

With continued reference to FIG. 5A, detecting signal 500a may include processing the signal 500a. For instance, apparatus 100 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further nonlimiting examples of algorithms that may be performed according to digital signal processing techniques include fast Fourier transform (FFT), Z-transform, finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIG. 5A, in one or more embodiments, detecting signal 500a and/or identifying one or more events therein comprises selecting an eligible period. For the purposes of this disclosure, an “eligible period” is a longer period of time during which signal 500a is generally stable (i.e., characterized by a stable baseline 512) and therefore worth looking for one or more signals; operationally, eligible period may be a period between two flanking, back-to-back flat intervals 504a-c and characterized by a very similar average from each of the flanking flat interval 504a-c. In one or more embodiments, the two flanking flat intervals 504a-c of eligible period may each have an average that is within +/−five times the expected noise of each other. For the purposes of this disclosure, an “expected noise” is the maximum deviation of signal 500a from its average within flat interval 504a-c. In one or more embodiments, the mean and standard deviation of each of the flanking intervals 504a-c may be further condensed to describe an entire eligible period. As a nonlimiting example, the mean of eligible period, μₑ, may be an average between the mean of the left flanking interval, μ_{L}, and mean of the right flanking interval, μ_{R}, whereas the standard deviation of eligible period, σₑ, may be the larger value between the standard deviation of the left flanking interval, σ_{L}, and standard deviation of the right flanking interval, σ_{R}.

Referring now to FIG. 5B, an exemplary embodiment 500b for an identified event 516, as described above, within signal 500a is illustrated. In one or more embodiments, event 516 may have a minimum height (i.e., detection threshold) 520 of 5×σₑ in order to be isolated from noise. Events may extend in either an upward or downward direction, depending on whether a positive or negative voltage is applied. In one or more embodiments, event 516 may include a start 524 and an end 528, the difference between which is the duration/width of the event 516, as described below. As a nonlimiting example, event 516 may have a minimum width of 0.1-0.3 milliseconds and/or a maximum width of 2-4 milliseconds. Event 516 may include a rising part, i.e., a portion of the event 516 from its onset to its peak and/or or portion of the event 516 where its first derivative meets a certain threshold. Similarly, event 516 may include a sinking par, i.e., a portion of the event 516 from its peak to its conclusion and/or a portion of the event 516 where its first derivative drops below a certain threshold. In one or more embodiments, event 516 may include a plurality of attributes that describe fine features of the event 516 from different perspectives, as described below.

Referring now to FIG. 5C, an exemplary embodiment 500c of several attributes that may be used to describe event 516 are illustrated. In one or more embodiments, event 516 may be described by a height attribute 532, as described above. For the purposes of this disclosure, a “height attribute” is an indicator that marks a maximum deviation of signal 500a from μₑ; “height attribute” and “intensity” may be used interchangeably throughout this disclosure. In some cases, height attribute may include a relative height attribute that is normalized with respect to the baseline; the use of such relative height attribute may reduce batch-to-batch variance between measurements. As a nonlimiting example, height attributes may first be normalized to relative height attributes before being used to train one or more machine-learning models. In one or more embodiments, event 516 may be described by a width attribute 536. For the purposes of this disclosure, a “width attribute” is an indicator that indicates a spatial or temporal span of event 516 based on detection threshold 520, as described above. In one or more embodiments, event 516 may be described by an area-under-the-curve attribute (AUC) 540. For the purposes of this disclosure, an “area-under-the-curve attribute” is an indicator that indicates an actual area beneath event 516 (i.e., the shaded area in FIG. 5C, which may be determined by performing an integral within spatial or temporal span of event 516) normalized with respect to a rectangular area defined by a product of height attribute 532 and width attribute 536. In one or more embodiments, event 516 may be described by an asymmetry attribute. For the purposes of this disclosure, an “asymmetry attribute” is an indicator that indicates a relative timestamp regarding where event 516 peaks at; it is reported as a ratio of a portion 544 of width attribute 536 wherein event 516 is ascending, with respect to the width attribute 536 of the entire event 516. As a nonlimiting example, when event 516 is symmetrical, asymmetry attribute has a benchmark value equal to 0.5; an asymmetry indicator smaller than 0.5 indicates that event 516 is skewed towards its right shoulder, whereas an asymmetry indicator larger than 0.5 indicates that event 516 is skewed towards its left shoulder instead. In one or more embodiments, event 516 may be described by a number-of-peaks attribute. For the purposes of this disclosure, a “number-of-peaks attribute” is the number of times event 516 rises above a peak threshold 548 near its peak value. As a nonlimiting example, peak threshold 548 may be set as 85% with respect to height attribute 532. In one or more embodiments, determining number-of-peaks attribute may involve a determination of the number of peaks or troughs within event 516, which may be accomplished by calculating the first derivative of the signal and determining the number of times the first derivative switches from positive to negative (for peaks) and/or from negative to positive (for troughs). Additionally, and/or alternatively, in one or more embodiments, other attributes besides those listed above may be used to describe the shape of event 516. As a nonlimiting example, an attribute may include a width-50 or full-width-at-half-maximum (FWHM) attribute, i.e., the width of event 516 at 50% of its peak height. Similarly, an attribute may include a width-10 attribute, a width-20 attribute, a width-30 attribute, and/or the like, that describes the width of event 516 at 10%, 20%, 30%, etc., of its peak height, respectively. As a nonlimiting example, an attribute may include a rising angle, i.e., the slope, steepness, or angle by which event 516 is rising. As another nonlimiting example, an attribute may include a sinking angle, i.e., the slope, steepness, or angle by which event 516 is sinking. Accordingly, as a further nonlimiting example, an attribute may include a rising AUC attribute, i.e., the AUC of the rising part of event 516, consistent with details described above. As a further nonlimiting example, these additional attributes may include a sinking AUC attribute, i.e., the AUC of the sinking part of event 516, consistent with details described above. As further nonlimiting examples, additional attributes may include without limitation peak-to-peak or trough-to-trough distance, frequency, absorbance, light attenuation at one or more wavelengths, or the like, as recognized by a person of ordinary skill in the art upon reviewing the entirety of this disclosure.

With continued reference to FIGS. 5A-C, in one or more embodiments, attributes described above pertaining to event 516 may be extracted after passing signal 500a through one or more low-pass, high-pass, and/or band-pass filters. In one or more embodiments, at least part of event 516 may be converted using a fast Fourier transform (FFT), Z-transform, Laplace transform, or the like, from a time domain to a frequency domain, wherein the at least part of event 516 may be dissected into a plurality of individual sine wave signals at different frequencies, and/or with different amplitudes, for further analysis and/or extraction of features; this transform may be accomplished with one or more machine-learning models, as described in this disclosure; in one or more embodiments, the transformed signal may be reverse-transformed to the time domain, after operations have been performed in the latent domain. A person of ordinary skill in the art, upon reviewing the entirety of this disclosure, will be able to recognize suitable attributes when analyzing event 516.

With continued reference to FIG. 5A-C, apparatus 100 may increase its certainty of classification by increasing the number of detected microbes 212. As a nonlimiting example, detection of a single microbe 212 may have a 70% certainty, which may not be satisfactory enough for medical applications; however, assuming a binomial distribution of probability, after the detection of 11 microbes, the probability of a correct classification increases to 92.2%; after detection of 51 microbes, this probability further increases to 99.86%; after detection of 100+ microbes, this probability will be virtually 100%. In addition, plurality of nanopores 104 of different sizes 404a-n, geometries 412a-n, and/or voltage differences 404a-n, as described above, when used in parallel, may also increase the certainty of detection, particularly for a mixture of microbes 212 of various types that may be more challenging to isolate and/or detect.

With continued reference to FIGS. 5A-C, in one or more embodiments wherein apparatus 100 includes plurality of nanopores 104, apparatus 100 may benefit from the multiplicity of the nanopores 104 by analyzing a plurality of signals 500a collected from different nanopores 104 simultaneously. A combined use of multiple nanopores 104 may result in a synergistic effect for the purpose of microbe identification. For example, and without limitation, when a first microbe 212 and a second microbe 212 are the only two microbes 212 capable of translocating through either a first nanopore 104 or a second nanopore 104, attributes of events 516 that they trigger may be indistinguishable. In some cases, it is by integrating height attribute 532 of a first event 516 collected from first nanopore 104 and one or more attributes of a second event 516 collected from second nanopore 104 that one may reliably identify two microbes 212 from each other.

With continued reference to FIG. 5A-C, signal 500a and/or event 516 may be either positive or negative, and similar synergies may arise from either type of signal 500a and/or event 516. Benefitting from the fact that some microbes 212 may not cross certain nanopores 104 under certain applied conditions, an absence of event 516 in some nanopores 104 may provide useful information that facilitates identification of microbe 212. For example, and without limitation, while both first microbe 212 and second microbe 212 may cross first nanopore 104, producing identically shaped events 516, only second microbe 212 may be able to crosse second nanopore 104. It may be only by considering both signals 500a and/or events 516 collected from both nanopores 104 simultaneously that one can reliably identify two microbes 212.

With continued reference to FIG. 5A-C, it is noteworthy that, while the computational pipeline may need to be performed by comparing signals 500a from multiple nanopores 104 simultaneously, signals 500a do not have to be recorded at the same time. For example, and without limitation, if a user has access to a single nanopore reader 116, the user may analyze a clinical sample multiple times by switching nanopore 104 and/or a condition applied thereto each time, as described above, thus effectively “simulating” a multiple-nanopore reader 116 in a serial rather than parallel fashion. Once recordings have been collected sequentially, the processing of all signals 500a may be carried out simultaneously to benefit from the synergy described above. Overall, apparatus 100 with plurality of nanopores 104, wherein the interpretation of event 516 in one nanopore 104 is conditioned by events 516 present in or absent from other nanopores 104, may have a significant advantage compared to single-nanopore designs.

With continued reference to FIG. 5A-C, it is worth noting that exemplary embodiments described herein are not the only possible ways to process signal 500a, identify events 516, and/or generate correlations. As a nonlimiting example, apparatus 100 may be configured to create embeddings for an entire signal 500a and extract one or more features therefrom using one or more classifiers, as described in this disclosure. A person of ordinary skill in the art, upon reviewing the entirety of this disclosure, will recognize additional variations of methods used herein for signal processing.

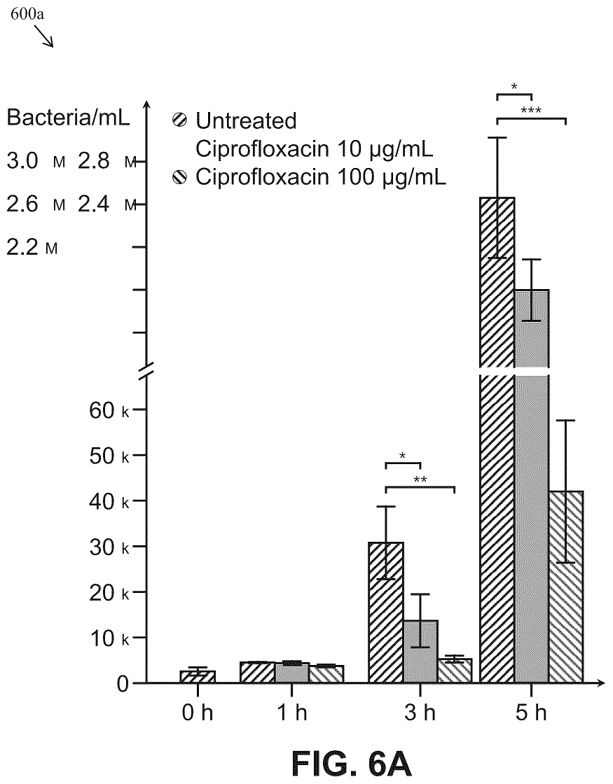

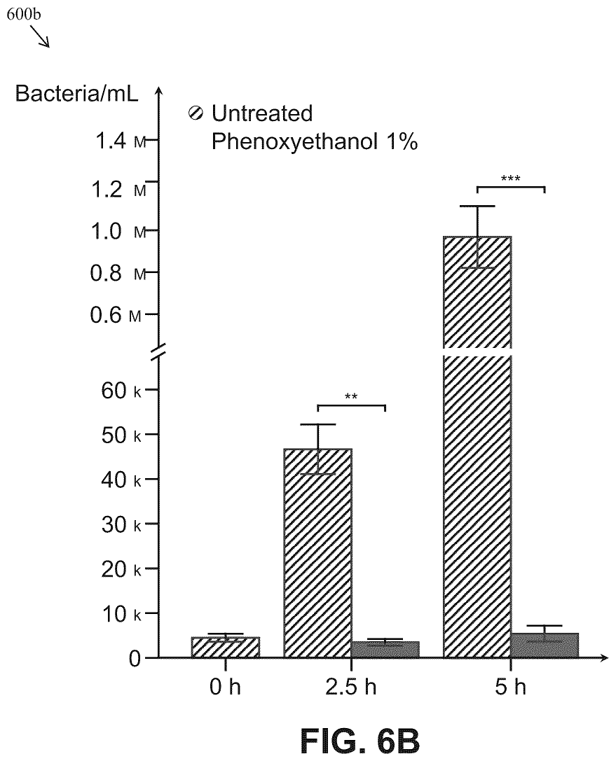

Referring now to FIG. 6A, an exemplary embodiment of experimental results 600a is illustrated to describe the use of apparatus 100 in a clinical context such as generation of an antibiogram. Experimental results 600a include bacteria concentrations of Escherichia coli measured with or without ciprofloxacin at various time delays. For the purposes of this disclosure, “ciprofloxacin” is a broad-spectrum antibiotic belonging to the fluoroquinolone class. The chemical structure of ciprofloxacin is 1-cyclopropyl-6-fluoro-1,4-dihydro-4-oxo-7-(1-piperazinyl)-3-quinolinecarboxylic acid, and it is commonly administered in the form of its hydrochloride salt. Ciprofloxacin is characterized by its ability to inhibit bacterial DNA gyrase and topoisomerase IV, which are enzymes critical for DNA replication, transcription, and repair in bacteria. The primary function of ciprofloxacin is to interfere with bacterial DNA synthesis, leading to the inhibition of cell division and bacterial growth. This mechanism of action makes ciprofloxacin effective against a wide range of Gram-negative and some Gram-positive bacteria. Due to its broad spectrum of activity, ciprofloxacin is commonly used to treat various bacterial infections, including urinary tract infections, respiratory tract infections, gastrointestinal infections, and skin infections. It may also be used in the treatment of certain types of bacterial infections that are resistant to other antibiotics. Due to the potential for developing antibiotic resistance, ciprofloxacin may not be abused and is typically reserved for use in infections where alternative treatments are not suitable or have failed.

With continued reference to FIG. 6A, it is worth noting that ciprofloxacin was simply chosen to provide a proof of concept, as apparatus 100 may be used to evaluate any type of antibiotics. Nonlimiting examples of broad-spectrum antibiotics other than ciprofloxacin may include fevofloxacin (a fluoroquinolone), moxifloxacin (a fluoroquinolone), amoxicillin-clavulanate (a penicillin with beta-lactamase inhibitor), ampicillin (a penicillin), piperacillin-tazobactam (a penicillin with beta-lactamase inhibitor), ceftriaxone (a third-generation cephalosporin), cefotaxime (a third-generation cephalosporin), cefepime (a fourth-generation cephalosporin), imipenem (a carbapenem), meropenem (a carbapenem), ertapenem (a carbapenem), tetracycline (a tetracycline), doxycycline (a tetracycline), minocycline (a tetracycline), azithromycin (a macrolide), clarithromycin (a macrolide), chloramphenicol (a amphenicol), tigecycline (a glycylcycline), and vancomycin (a glycopeptide), among others. Similarly, apparatus 100 may be used to generate an antibiogram of any bacteria and/or otherwise evaluate any antimicrobial resistance (AMR) or multiple-drug resistance (MDR) associated thereto, as recognized by a person of ordinary skill in the art upon reviewing the entirety of this disclosure.

With continued reference to FIG. 6A, in order to record these results, a sample of Escherichia coli was split into three falcon tubes, tube A (for sample 1), tube B (for sample 2), and tube C (for sample 3). Tube A only contained a growth medium and served as the control group. Tube B contained the same growth medium alongside 10 μg/mL ciprofloxacin, which is the recommended dosage for lab work on bacteria, although the efficacy of such dosage may depend on a multitude of factors, such as without limitation bacterial concentration, bacterial strain, temperature, among others. Tube C contained the same growth medium alongside 100 μg/mL of ciprofloxacin, which is a large dosage that is ten times the recommended dosage. These two dosages were selected as benchmarks to evaluate how bacteria respond to the stress of antibiotics. All three samples were measured after 0 h, 1 h, 3 h, and 5 h of incubation, under the same conditions, and all samples showed monotonic growth in bacterial concentration over time. The growth of bacteria slowed down as the dosage of ciprofloxacin increased. The mean bacterial concentration and the standard deviation (WSTD) associated thereto are tabulated in Table 1 below. Bacterial concentrations described herein were determined by converting the density of events 516 (i.e., the number of events 516 per second) using the Poiseuille equation, based on a hydrostatic pressure differential, Δp, of 30 mmH₂O.

TABLE 1
Experimental Results for Bacterial Concentrations Measured
Using Escherichia coli in Growth Media Containing No
Treatment, A Treatment with 10 μg/mL Ciprofloxacin,
and with a Treatment with 100 μg/mL Ciprofloxacin,
Respectively, after 0 h, 1 h, 3 h, and 5 h of Incubation.

| | mean | WSTD |
|---|---|---|
| 0 h/untreated | 2,168.84 | 895.82 |
| 1 h/untreated | 4,455.48 | 130.56 |
| 1 h/ciprofloxacin, 10 ug/mL | 4,366.50 | 449.57 |
| 1 h/ciprofloxacin, 100 ug/mL | 3,805.50 | 356.83 |
| 3 h/untreated | 30,678.90 | 7,930.99 |
| 3 h/ciprofloxacin, 10 ug/mL | 13,645.19 | 5,838.79 |
| 3 h/ciprofloxacin, 100 ug/mL | 5,315.36 | 753.94 |
| 5 h/untreated | 2,830,666.89 | 280,334.34 |
| 5 h/ciprofloxacin, 10 ug/mL | 2,398,725.42 | 143,314.16 |
| 5 h/ciprofloxacin, 100 ug/mL | 42,020.06 | 15,663.85 |

Referring now to FIG. 6B, an exemplary embodiment of experimental results 600b is illustrated to describe the use of apparatus 100 in a nonclinical context such as preservation of food or cosmetics. Experimental results 600b include bacterial concentrations measured with or without phenoxyethanol at various time delays. For the purposes of this disclosure, “phenoxyethanol” is an organic compound that often serves as a preservative in cosmetics, pharmaceuticals, and personal care products. It is a colorless, oily liquid with a mild rose-like scent and is chemically classified as a glycol ether. The chemical structure of phenoxyethanol is C₈H₁₀O₂ or C₆H₅OC₂H₄OH. Phenoxyethanol functions primarily as an antimicrobial agent, helping to prevent the growth of bacteria, yeast, and mold in formulations.

With continued reference to FIG. 6B, it is worth noting that phenoxyethanol was simply chosen to provide a proof of concept, as apparatus 100 may be used to evaluate any type of preservative in any composition of matter or formulation such food, cosmetics, or the like, as recognized by a person of ordinary skill in the art upon reviewing the entirety of this disclosure. Nonlimiting examples of preservatives other than phenoxyethanol may include benzyl alcohol, sodium benzoate, potassium sorbate, sorbic acid, ethylhexylglycerin, diazolidinyl urea, imidazolidinyl urea, chlorphenesin, caprylyl glycol, ethylenediaminetetraacetic acid (EDTA) including its tetrasodium salt, butylated hydroxytoluene (BHT), formaldehyde-releasing preservatives including DMDM hydantoin and quaternium-15, methylisothiazolinone, and methylchloroisothiazolinone, among others.

With continued reference to FIG. 6B, in order to record these records, a sample of Escherichia coli was split into two falcon tubes, tube A (for sample 4) and tube B (for sample 5). Tube A only contained a growth medium and served as the control group, whereas tube B contained the same growth medium alongside 1% phenoxyethanol, which is the maximum allowed concentration for phenoxyethanol to be used as a preservative in consumer products. Both samples were measured after 0 h, 2.5 h, and 5 h of incubation, under the same conditions. While sample 4 showed a monotonic growth in bacterial concentration over time, the bacterial concentration in sample 5 remained stable due to the presence of phenoxyethanol. The mean bacterial concentration and the standard deviation (WSTD) associated thereto are tabulated in Table 2 below.

TABLE 2
Experimental Results for Bacterial Concentrations
Measured Using Escherichia coli in Growth Media
Containing No Treatment and 1% Phenoxyethanol, Respectively,
after 0 h, 2.5 h, and 5 h of Incubation.

| | mean | WSTD |
|---|---|---|
| 0 h/untreated | 4,473.37 | 868.87 |
| 2.5 h/untreated | 46,628.08 | 5,575.81 |
| 2.5 h/phenoxyethanol | 3,516.33 | 682.43 |
| 5 h/untreated | 969,588.78 | 118,240.09 |
| 5 h/phenoxyethanol | 5,446.70 | 1,802.26 |

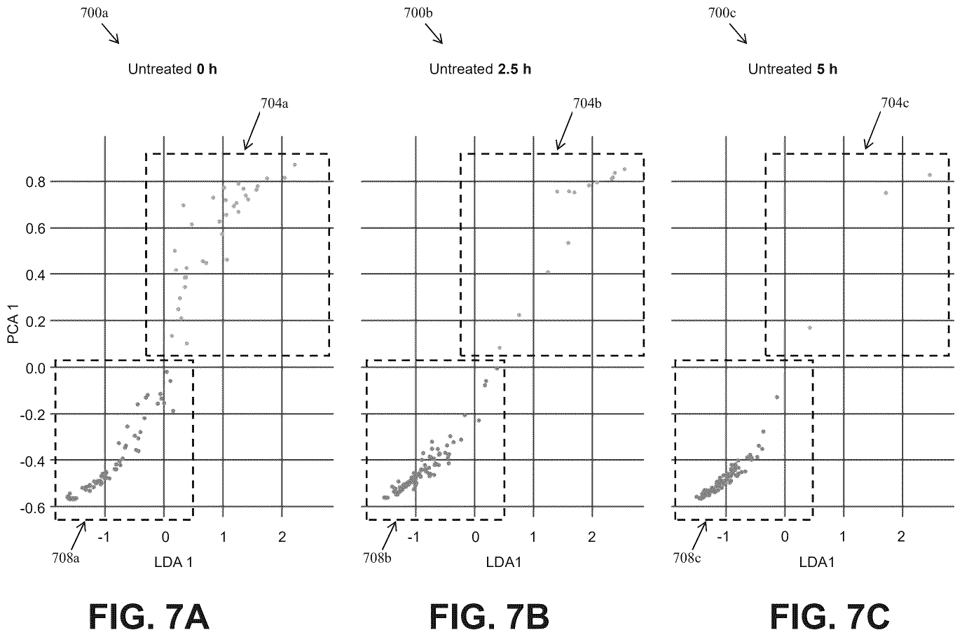

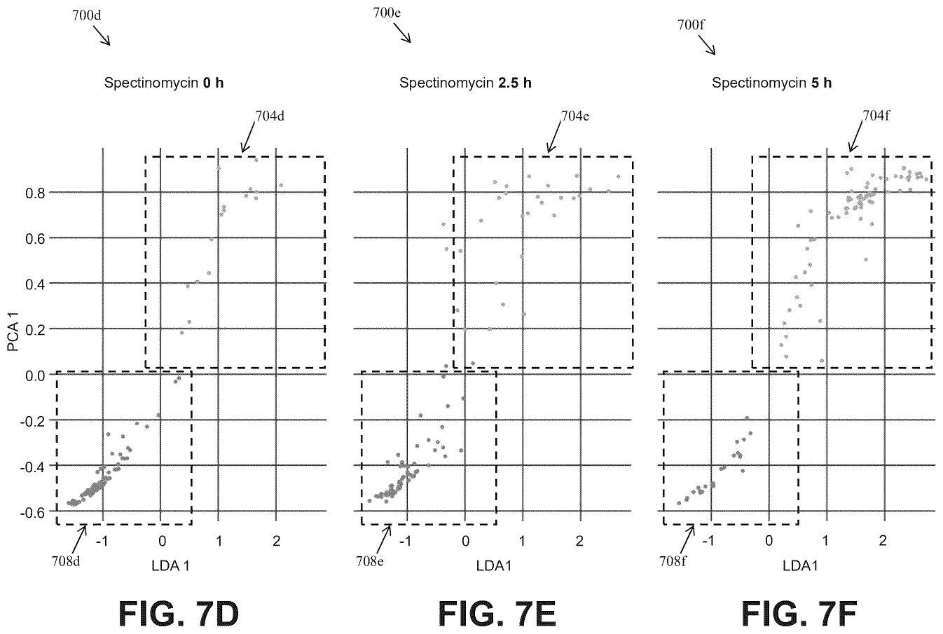

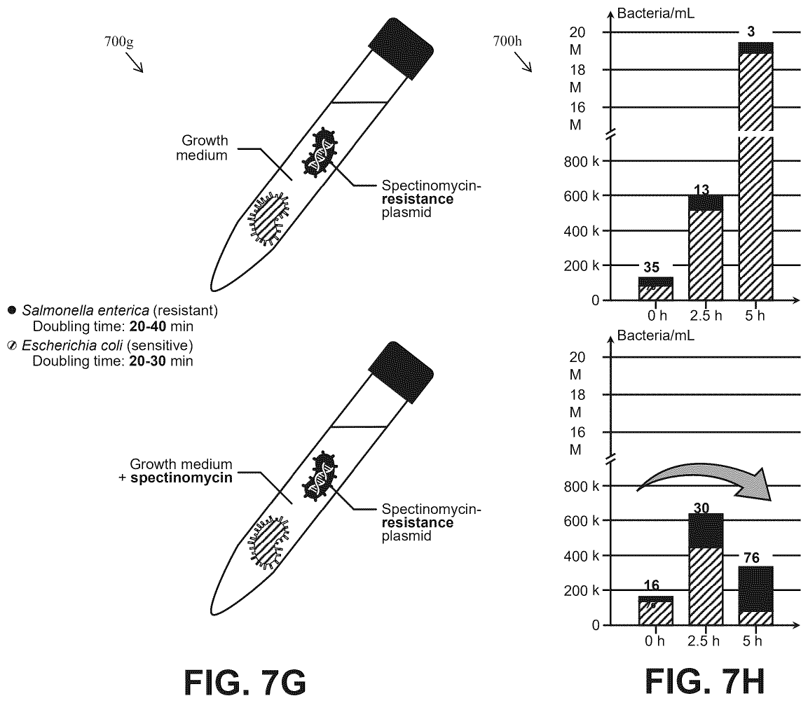

Referring now to FIGS. 7A-F, nonlimiting examples of correlation datasets 700a-f are illustrated. Correlation datasets 700a-f are generated based on signals/events that are measured using a mixture of spectinomycin-sensitive Escherichia coli and spectinomycin-resistant Salmonella enterica, after 0 h, 2.5 h, and 5 h of incubation. For the purposes of this disclosure, “spectinomycin” is an antibiotic that belongs to the aminocyclitol class of antibiotics, which are structurally related to aminoglycosides. Spectinomycin is derived from the bacterium Streptomyces spectabilis and is primarily used for the treatment of gonorrhea, particularly in cases where other antibiotics, such as penicillin, are not effective due to resistance or allergies. The mechanism of action of spectinomycin involves inhibiting protein synthesis in bacteria by binding to the 30S ribosomal subunit, thereby interfering with the translation process. Unlike aminoglycosides, spectinomycin does not cause misreading of the genetic code, but it effectively halts bacterial growth by preventing the elongation of the polypeptide chain.

With continued 50 ug microbes 212 increases, different clustering patterns of data points, 704a-f for spectinomycin-resistant Salmonella enterica and 708a-f for spectinomycin-sensitive Escherichia coli, were observed; this difference may be a result of different sizes, different shapes, and/or different surface charges of Escherichia coli vs. Salmonella enterica, which results in different transport behaviors when a bacterium of either type travels through nanopore 104. Accordingly, spectinomycin-sensitive Escherichia coli and spectinomycin-resistant Salmonella enterica may be identified based on such clustering patterns.

Referring now to FIG. 7G, an exemplary embodiment 700g of an experimental protocol is illustrated. Such experimental protocol may be used to culture spectinomycin-resistant Salmonella enterica and spectinomycin-sensitive Escherichia coli and generate data in FIGS. 7A-F. The wild strains of both Escherichia coli and Salmonella enterica are sensitive to spectinomycin. However, in order to illustrate the use of apparatus 100 in generating antibiograms, a pair of microbes that differ in both identity and drug resistance may be used as a more suitable proof of concept. Therefore, a plasmid that contains a gene conveying spectinomycin resistance was introduced to Salmonella enterica to make it spectinomycin-resistant. A mixture of spectinomycin-resistant Salmonella enterica and spectinomycin-sensitive Escherichia coli are subsequently incubated in suitable growth media, under the same conditions, for 0 h, 2.5 h, and 5 h, and measurements were taken at each time point. Sample 6 did not contain spectinomycin and served as the control group, whereas sample 7 contained spectinomycin at a dosage of 50 μg/mL.

Referring now to FIG. 7H, an exemplary embodiment 700h of experimental results for bacterial concentrations measured based on FIGS. 7A-F are illustrated. Bacterial concentrations described herein were determined by converting the density of events 516 (i.e., the number of events 516 per second) using the Poiseuille equation, based on a hydrostatic pressure differential, Δp, of 0.8 mmH₂O. It is proposed that a relatively small Δp (e.g., less than 5 mmH₂O) may help improve the accuracy of classification in a mixed population of microbes 212, thereby ensuring a simultaneous identification and quantification of the microbes 212 with a high fidelity. Embodiment 700h describes quantification results based on identification results described in FIGS. 7A-F. Based on the correlation datasets in FIGS. 7A-F, events resulting from spectinomycin-resistant Salmonella enterica and spectinomycin-sensitive Escherichia coli are classified and tallied separately to generate growth curves, consistent with details described above in this disclosure. For sample 6, the total concentration of bacteria appeared to increase monotonically from 0 h to 5 h. In contrast, the total concentration of bacteria in sample 7 increased in the first 2.5 h, then decreased in the second 2.5 h. However, it is worth noting the change in bacterial concentration for spectinomycin-resistant Salmonella enterica and spectinomycin-sensitive Escherichia coli individually. In sample 6, spectinomycin-sensitive Escherichia coli outcompeted spectinomycin-resistant Salmonella enterica, with the percentage of the spectinomycin-sensitive Escherichia coli increasing from 65% to 97% and the percentage of the spectinomycin-resistant Salmonella enterica decreasing from 35% to 3%. In contrast, in sample 7, the population of spectinomycin-resistant Salmonella enterica gradually replaced the population of spectinomycin-sensitive Escherichia coli, with the percentage of the spectinomycin-resistant Salmonella enterica expanding from 16% to 76%. In addition, in sample 7, the net bacterial concentration for spectinomycin-sensitive Escherichia coli increased in the first 2.5 h, then decreased in the second 2.5 h, while the net bacterial concentration increased monotonically for spectinomycin-resistant Salmonella enterica. In other words, upon supplying a selective stress of spectinomycin, spectinomycin-resistant Salmonella enterica survived, whereas spectinomycin-sensitive Escherichia coli decayed.

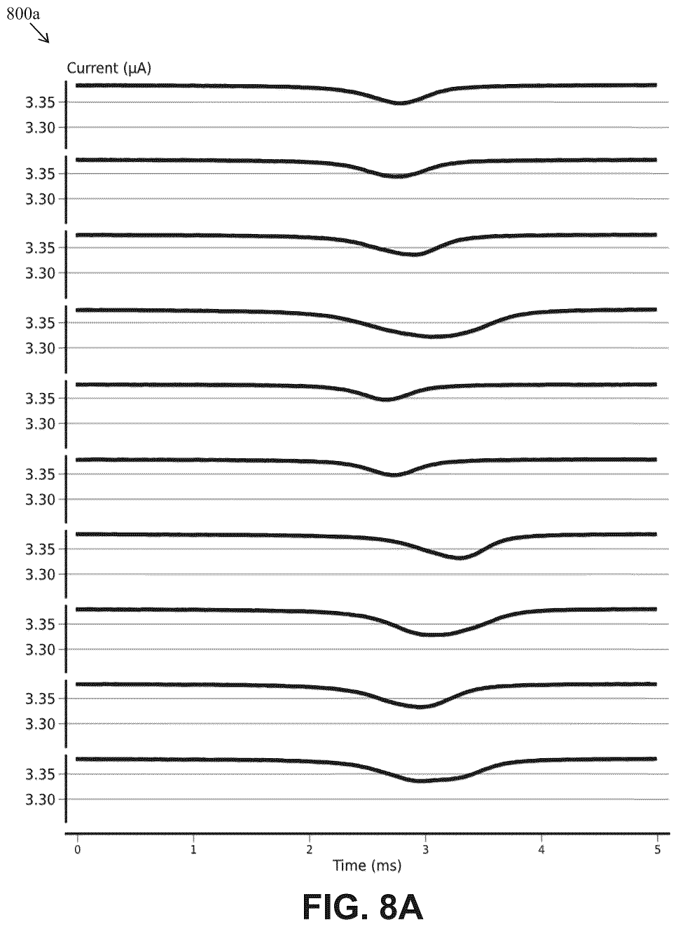

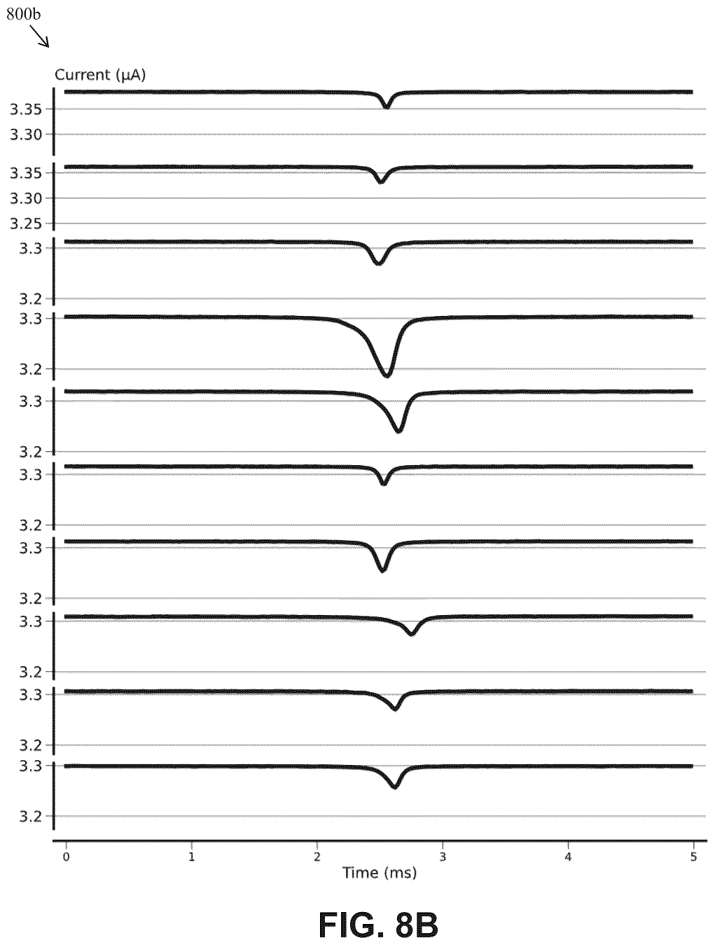

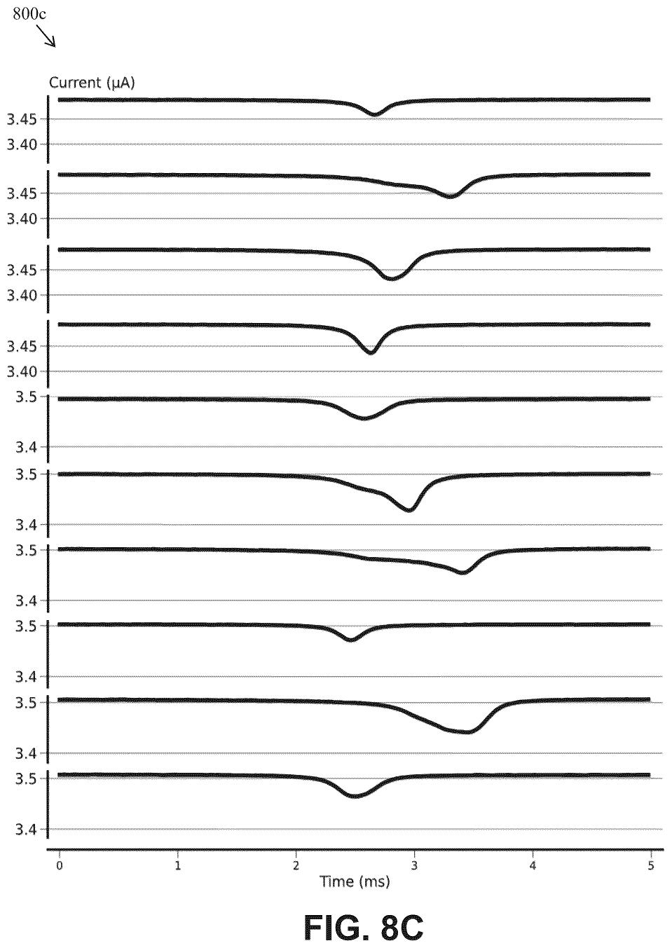

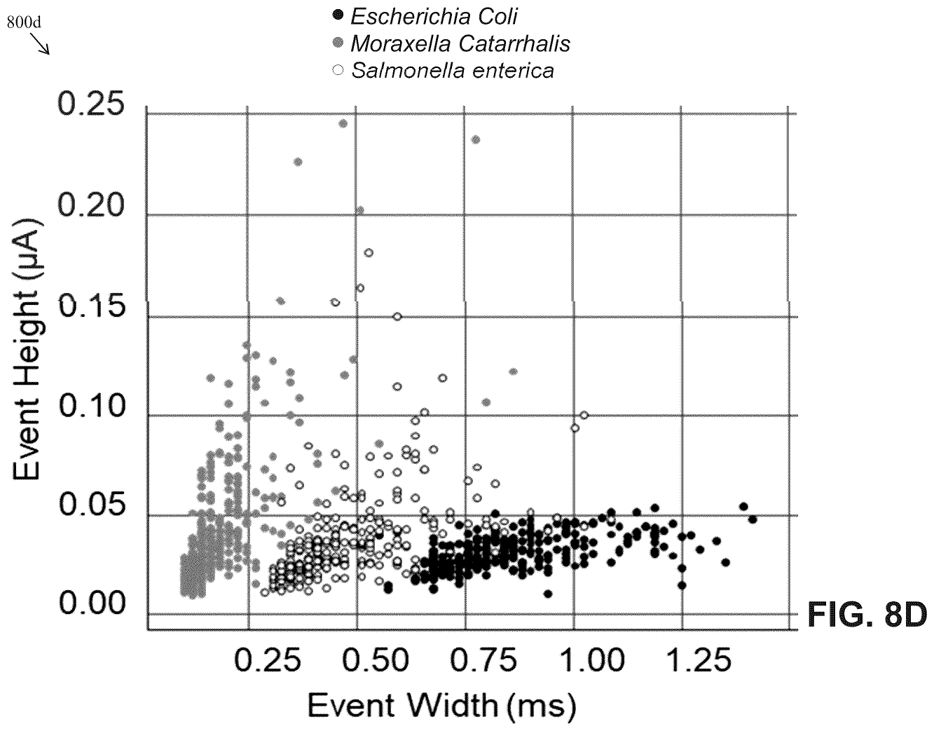

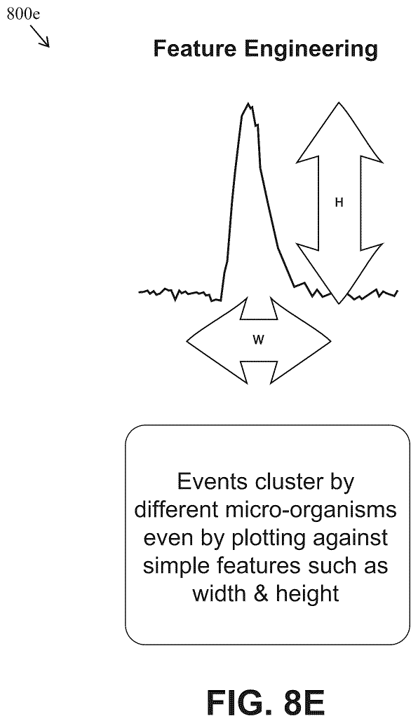

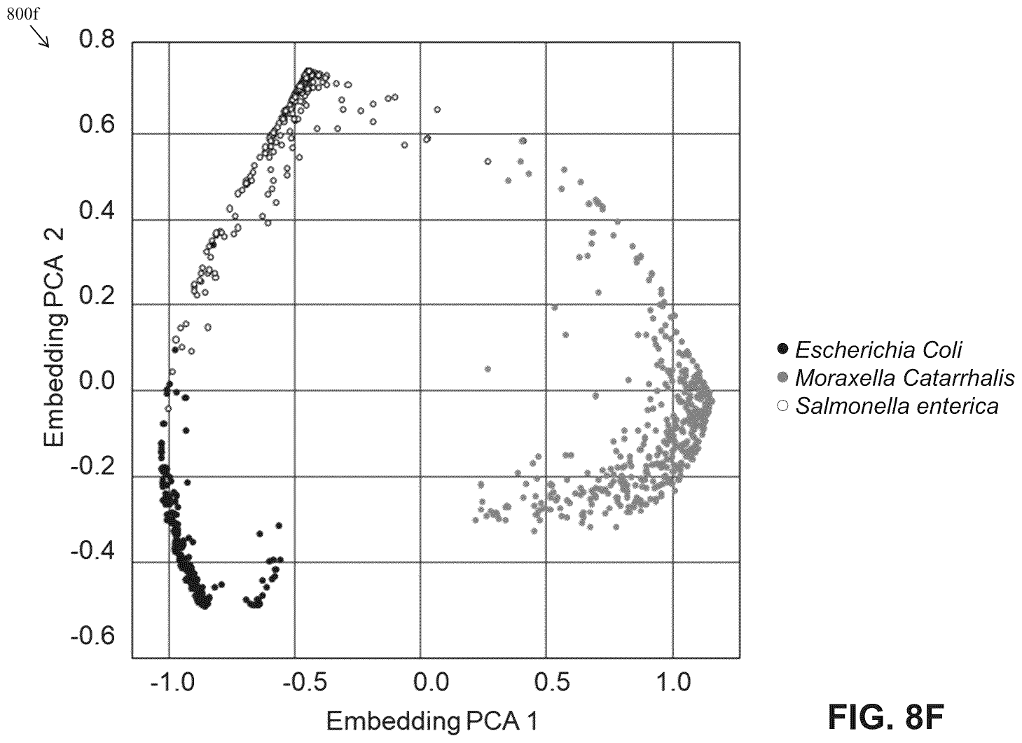

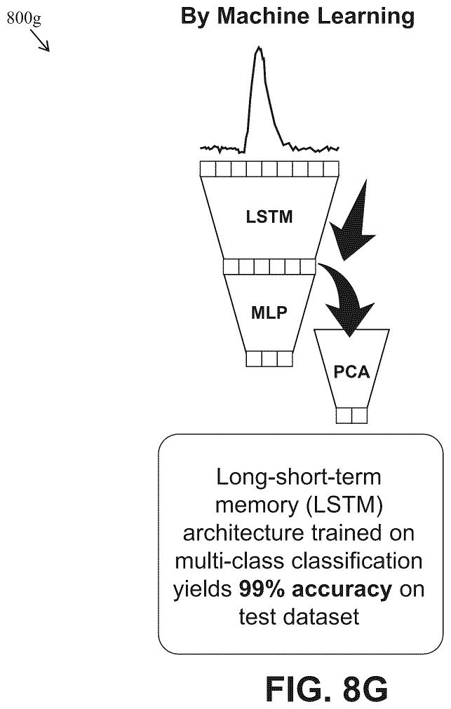

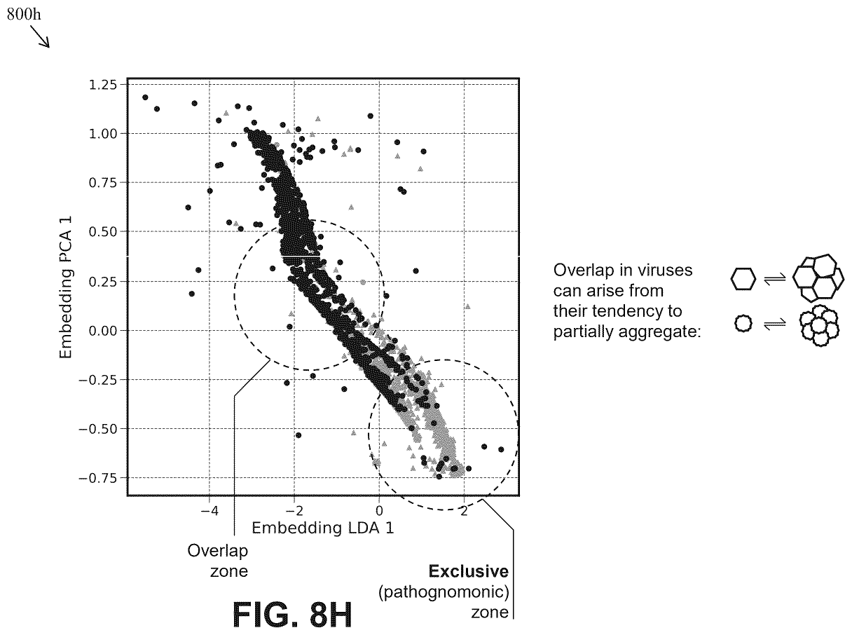

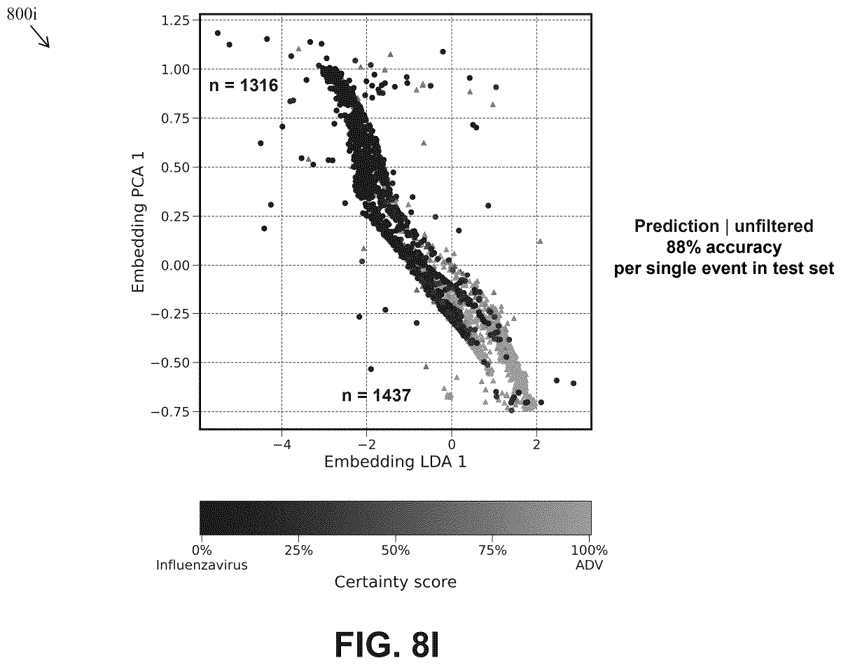

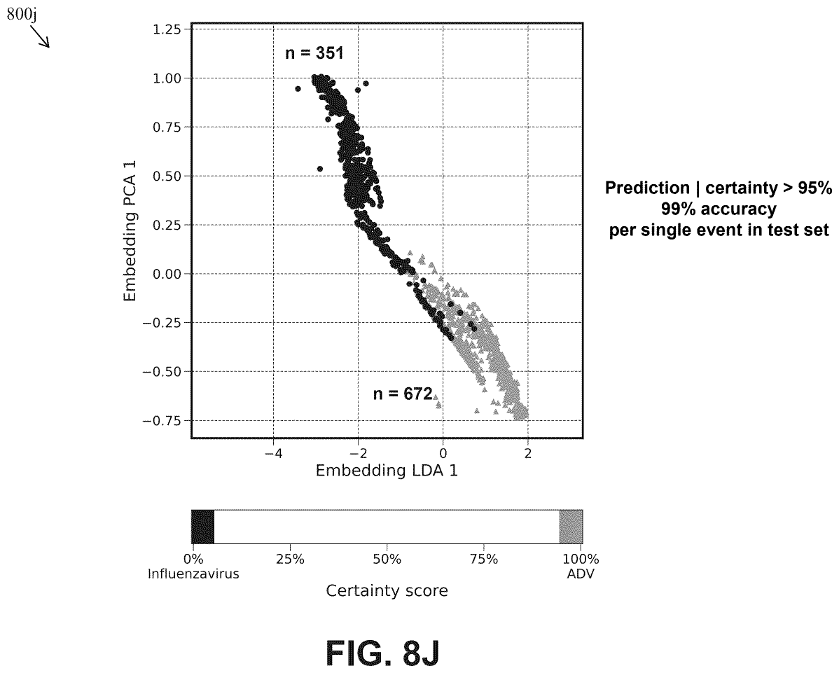

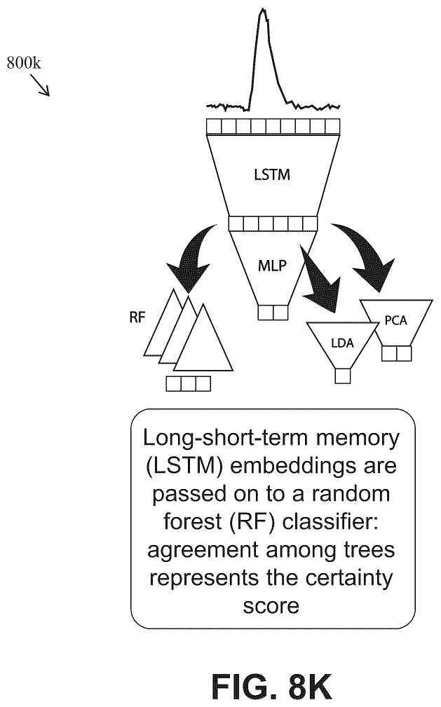

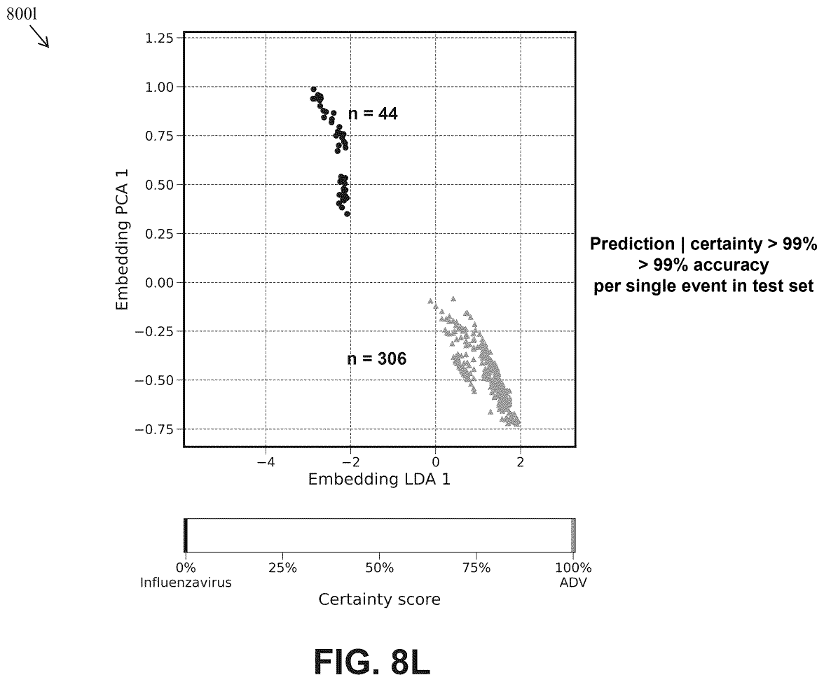

Referring now to FIGS. 8A-C, exemplary embodiments 800a-c of resistive curves are illustrated, consistent with details described above in this disclosure. These resistive curves are collected by apparatus 100 using Escherichia coli, Moraxella catarrhalis, and Salmonella enterica. The horizontal axis represents a time delay measured in milliseconds (ms), whereas the vertical axis represents the current through nanopore 104 in microamps (μA). Translocation of microbe 212 across nanopore 104 may result in a temporary displacement of conductive, ionic species within the nanopore 104, which causes a reduction of conductivity and results in a resistive pulse in the shape of a negative peak. Events resulting from such negative peaks are highlighted using boxes.

Referring now to FIGS. 8D-E, FIG. 8D is an exemplary embodiment 800d of correlation plots between a width attribute and a height attribute based on events in FIGS. 8A-C, consistent with details described above in FIGS. 5A-C. An exemplary embodiment 800e of a feature-learning process that may be used to generate such correlation plots is illustrated in FIG. 8E. The solid dark symbols, solid gray symbols, and hollow symbols represent Escherichia coli, Moraxella catarrhalis, and Salmonella enterica, respectively. Correlations between the width and height of events form distinct clusters for each of three microbes; such differences may form the basis of identification in certain embodiments.

Referring now to FIG. 8F-G, FIG. 8F is an exemplary embodiment 800f of correlation plots generated using LSTM, MLP, and/or PCA, based on data in FIGS. 8A-C. Specifically, events 516 (i.e., raw sequences”) are first detected, consistent with details described above in this disclosure. LSTM and MLP are subsequently applied to transform these raw sequences of variable lengths into embeddings of a fixed length (for example and without limitation, a fixed length of 32). MLP then makes label predictions for test dataset entries as a function of these embeddings. Subsequently, PCA is applied to these embeddings. Correlation plots are then created by applying PCA to generate both the horizontal and the vertical axes. The correlations of the three types of microbes form three distinct clusters, which may provide a basis for the detection mechanism of apparatus 100, consistent with details described above. Additionally, an exemplary embodiment 800g of a machine-learning architecture that may be used to generate such correlation plots is illustrated in FIG. 8G. Specifically, a long-short-term memory (LSTM) architecture trained on multi-class classification, when combined with PCA, may yield a 99% accuracy on a test dataset.

Referring now to FIGS. 8H-L, FIGS. 8H, I, J, and L are exemplary embodiments 800h, 800i, 800j, and 800l of correlation plots generated using LSTM, MLP, a random forest (RF) classifier, PCA, and/or LDA. These embodiments are generated using data measured from Influenzavirus type A and Adenovirus type 5 with overlapping correlations and showcase scenarios where more advanced data processing techniques may be needed for apparatus 100. Additionally, an exemplary embodiment 800k of a machine-learning architecture that may be used to generate such correlation plots is illustrated in FIG. 8K.

With continued reference to FIGS. 8H-L, FIG. 8H includes a correlation plot generated using a combination of PCA (for the vertical axis) and LDA (for the horizontal axis). This correlation plot contains not only an exclusive (i.e., pathognomonic) zone for each type of virus, but also an overlap zone between the two types of viruses. Such overlap may arise due to a tendency of viruses to aggregate, and these aggregates may potentially obscure certain unique features in individual viruses and make these viruses more challenging to resolve. Using unfiltered data that contain 1316 events 516 from Influenzavirus type A and 1437 events 516 from Adenovirus type 5, apparatus 100 may determine the identity of a virus with an 88% accuracy per single event in a test set, as shown in FIG. 8I. The certainty score of these identifications also shows a wide distribution between 0% and 100%. To improve the accuracy of apparatus 100 in identification of microbes, LSTM embeddings, alongside hard-coded features described above (e.g., relative height, width, AUC, asymmetry, multiplicity, and/or the like), may be passed on to an ensemble model including a plurality of independent, parallelized classifiers. Such ensemble model may include any type of ensemble model or classifier architecture described in this disclosure or otherwise recognized by a person of ordinary skill in the art upon reviewing the entirety of this disclosure. In some cases, such ensemble model may include without limitation an RF or an ensemble of decision trees similar thereto, an ensemble of support vector machines, an ensemble of XGBoost models, and/or an ensemble of MLPs; accordingly, agreement among plurality of independent classifiers within the ensemble model may represent a certainty score. As a nonlimiting example, an ensemble model may include a plurality of classifiers of mixed types. As another nonlimiting example, an ensemble model may include 10-500 classifiers. Accordingly, events 516 may be filtered based on such a certainty score by selecting a cutoff at 95%, 99%, or the like.

With continued reference to FIGS. 8H-L, FIG. 8J includes an updated correlation plot using filtered data that contain 351 events 516 from Influenzavirus type A and 672 events 516 from Adenovirus type 5, based on a cutoff in certainty score at 95%. Correlations for Influenzavirus type A and Adenovirus type 5 are now better resolved with less overlap in between, and accordingly, apparatus 100 may determine the identity of a virus with a 99% accuracy per single event in a test set. The accuracy of apparatus 100 may be further improved by selecting a more stringent cutoff. FIG. 8L includes a further updated correlation plot using filtered data that contain 44 events 516 from Influenzavirus type A and 306 events 516 from Adenovirus type 5, based on a cutoff in certainty score at 99%. Correlations for Influenzavirus type A and Adenovirus type 5 are now completely resolved with no visible overlap in between, and accordingly, apparatus 100 may now determine the identity of a virus with a >99% accuracy per single event in a test set. Additional details pertaining to RF will be provided below in this disclosure. Similarly, such filtering process may be applied to quantification of microbe 212. Specifically, a concentration of microbe may be quantified based on the number of events 516 that remains after filtering the original pool of events 516 based on a certain certainty score/accuracy requirement and an estimated percentage of events that remains after such filtering step. As a nonlimiting example, a pathognomonic region in the embedding space may be filtered to achieve a >99% specificity for Salmonella enterica while losing 75% of the events. Therefore, if after such filtering step, a sample is determined to contain 1 M bacteria/mL in the pathognomonic area for Salmonella enterica, then, through back-calculation, it may be determined that the sample contains 4 M Salmonella enterica/mL.

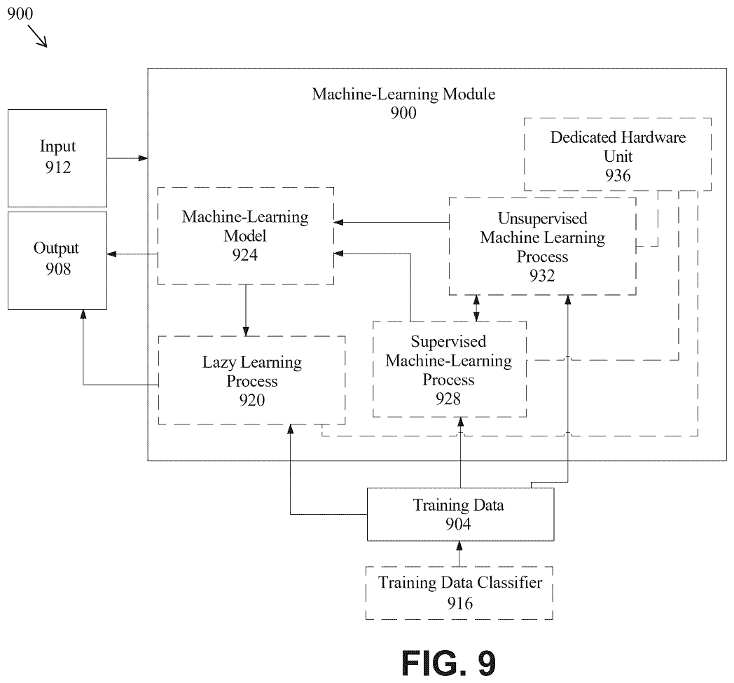

Referring now to FIG. 9, an exemplary embodiment of a machine-learning module 900 that may perform one or more machine-learning processes as described above is illustrated. Machine-learning module may perform determinations, classification, and/or analysis steps, methods, processes, or the like as described in this disclosure using machine-learning processes. For the purposes of this disclosure, a “machine-learning process” is an automated process that uses training data 904 to generate an algorithm instantiated in hardware or software logic, data structures, and/or functions that will be performed by a computing device/module to produce outputs 908 given data provided as inputs 912. This is in contrast to a non-machine-learning software program where the commands to be executed are pre-determined by user and written in a programming language.

With continued reference to FIG. 9, “training data”, for the purposes of this disclosure, are data containing correlations that a machine-learning process uses to model relationships between two or more categories of data elements. For instance, and without limitation, training data 904 may include a plurality of data entries, also known as “training examples”, each entry representing a set of data elements that were recorded, received, and/or generated together. Data elements may be correlated by shared existence in a given data entry, by proximity in a given data entry, or the like. Multiple data entries in training data 904 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 904 according to various correlations; correlations may indicate causative and/or predictive links between categories of data elements, which may be modeled as relationships such as mathematical relationships by machine-learning processes as described in further detail below. Training data 904 may be formatted and/or organized by categories of data elements, for instance by associating data elements with one or more descriptors corresponding to categories of data elements. As a nonlimiting example, training data 904 may include data entered in standardized forms by persons or processes, such that entry of a given data element within a given field in a given form may be mapped to one or more descriptors of categories. Elements in training data 904 may be linked to descriptors of categories by tags, tokens, or other data elements. For instance, and without limitation, training data 904 may be provided in fixed-length formats, formats linking positions of data to categories such as comma-separated value (CSV) formats, tab-separated values (.TSV), Axon Binary Format (.ABF), Pickle (.PICKLE), Joblib (.JL), and/or self-describing formats such as extensible markup language (XML), JavaScript Object Notation (JSON), or the like, enabling processes or devices to detect categories of data.

With continued reference to FIG. 9, alternatively, or additionally, training data 904 may include one or more elements that are uncategorized; that is, training data 904 may not be formatted or contain descriptors for some elements of data. Machine-learning algorithms and/or other processes may sort training data 904 according to one or more categorizations using, for instance, natural language processing algorithms, tokenization, detection of correlated values in raw data, and the like; categories may be generated using correlation and/or other processing algorithms. As a nonlimiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 904 to be made applicable for two or more distinct machine-learning algorithms as described in further detail below. Training data 904 used by machine-learning module 900 may correlate any input data as described in this disclosure to any output data as described in this disclosure. As a nonlimiting illustrative example, inputs may include inputs such as training signals and the like, and outputs may include outputs such as one or more correlations between attributes and extracted features or patterns therefrom.

With continued reference to FIG. 9, training data 904 may be filtered, sorted, and/or selected using one or more supervised and/or unsupervised machine-learning processes and/or models as described in further detail below; such processes and/or models may include without limitation a training data classifier 916. For the purposes of this disclosure, a “classifier” is a machine-learning model that sorts inputs into categories or bins of data, outputting the categories or bins of data and/or labels associated therewith. Machine-learning model may include without limitation a data structure representing and/or using a mathematical model, neural net, or a program generated by a machine-learning algorithm, known as a “classification algorithm”. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 900 may generate a classifier using a classification algorithm. For the purposes of this disclosure, a “classification algorithm” is a process wherein a computing device and/or any module and/or component operating therein derives a classifier from training data 904. Classification may be performed using, without limitation, linear classifiers such as without limitation logistic regression and/or naive Bayes classifiers, nearest neighbor classifiers such as k-nearest neighbors classifiers, XGBoost, LDA, support vector machines, least squares support vector machines, Fisher's linear discriminant, quadratic classifiers, decision trees, boosted trees, random forest classifiers, learning vector quantization, and/or neural network-based classifiers. In one or more embodiments, training data classifier 916 may classify elements of training data to a plurality of cohorts as a function of certain features or traits.

With continued reference to FIG. 9, machine-learning module 900 may be configured to generate a classifier using a naive Bayes classification algorithm. Naive Bayes classification algorithm generates classifiers by assigning class labels to problem instances, represented as vectors of element values. Class labels are drawn from a finite set. Naive Bayes classification algorithm may include generating a family of algorithms that assume that the value of a particular element is independent of the value of any other element, given a class variable. Naive Bayes classification algorithm may be based on Bayes Theorem expressed as P(A/B)=P(B/A)×P(A)÷P(B), where P(A/B) is the probability of hypothesis A given data B, also known as posterior probability; P(B/A) is the probability of data B given that the hypothesis A was true; P(A) is the probability of hypothesis A being true regardless of data, also known as prior probability of A; and P(B) is the probability of the data regardless of the hypothesis. A naive Bayes algorithm may be generated by first transforming training data into a frequency table. Machine-learning module 900 may then calculate a likelihood table by calculating probabilities of different data entries and classification labels. Machine-learning module 900 may utilize a naive Bayes equation to calculate a posterior probability for each class. A class containing the highest posterior probability is the outcome of prediction. Naive Bayes classification algorithm may include a gaussian model that follows a normal distribution. Naive Bayes classification algorithm may include a multinomial model that is used for discrete counts. Naive Bayes classification algorithm may include a Bernoulli model that may be utilized when vectors are binary.

With continued reference to FIG. 9, machine-learning module 900 may be configured to generate a classifier using a k-nearest neighbors (KNN) algorithm. For the purposes of this disclosure, a “k-nearest neighbors algorithm” is or at least includes a classification method that utilizes feature similarity to analyze how closely out-of-sample features resemble training data 904 and to classify input data to one or more clusters and/or categories of features as represented in training data 904. This may be performed by representing both training data 904 and input data in vector forms and using one or more measures of vector similarity to identify classifications within training data 904 and determine a classification of input data. K-nearest neighbors algorithm may include specifying a k-value, or a number directing the classifier to select the k most similar entries of training data 904 to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a nonlimiting example, an initial heuristic may include a ranking of associations between inputs 912 and elements of training data 904. Heuristic may include selecting some number of highest-ranking associations and/or training data elements.

With continued reference to FIG. 9, generating k-nearest neighbors algorithm may generate a first vector output containing a data entry cluster, generating a second vector output containing input data, and calculate the distance between the first vector output and the second vector output using any suitable norm such as cosine similarity, Euclidean distance measurement, or the like. Each vector output may be represented, without limitation, as an n-tuple of values, where n is at least 2. Each value of n-tuple of values may represent a measurement or other quantitative value associated with a given category of data or attribute, examples of which are provided in further detail below. A vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent when their directions and/or relative quantities of values are the same; thus, as a nonlimiting example, a vector represented as [5, 10, 15] may be treated as equivalent, for the purposes of this disclosure, as a vector represented as [1, 2, 3]. Vectors may be more similar where their directions are more similar, and more different where their directions are more divergent. However, vector similarity may alternatively, or additionally, be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized”, or divided by a “length” attribute, such as a length attribute l as derived using a Pythagorean norm:

$$l = \sqrt{{\sum }_{i = 0}^{n} {a}_{i}^{2}} ,$$

where aᵢ is attribute number of vector i. Scaling and/or normalization may function to make vector comparison independent of absolute quantities of attributes, while preserving any dependency on similarity of attributes. This may, for instance, be advantageous where cases represented in training data 904 are represented by different quantities of samples, which may result in proportionally equivalent vectors with divergent values.

With continued reference to FIG. 9, training examples for use as training data may be selected from a population of potential examples according to cohorts relevant to an analytical problem to be solved, a classification task, or the like. Alternatively, or additionally, training data 904 may be selected to span a set of likely circumstances or inputs for a machine-learning model and/or process to encounter when deployed. For instance, and without limitation, for each category of input data to a machine-learning model and/or process that may exist in a range of values in a population of phenomena such as images, user data, process data, physical data, or the like, a computing device, control unit 132, and/or machine-learning module 900 may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively, or additionally, a set of training examples may be compared to a collection of representative values in a database and/or presented to user, so that a process can detect, automatically or via user input, one or more values that are not included in the set of training examples. Computing device, control unit 132, and/or machine-learning module 900 may automatically generate a missing training example. This may be done by receiving and/or retrieving a missing input and/or output value and correlating the missing input and/or output value with a corresponding output and/or input value collocated in a data record with the retrieved value, provided by user, another device, or the like.

With continued reference to FIG. 9, computing device, control unit 132, and/or machine-learning module 900 may be configured to preprocess training data 904. For the purposes of this disclosure, “preprocessing” training data is a process that transforms training data from a raw form to a format that can be used for training a machine-learning model. Preprocessing may include sanitizing, feature selection, filtering (low-pass, high-pass, band-pass or any combination of multiple filters), operations in the Fourier/Laplace/Z-domain (i.e., transforming the trace, applying the operation, and reverse-transforming to the time domain), feature scaling, data augmentation, and the like.

With continued reference to FIG. 9, computing device, control unit 132, and/or machine-learning module 900 may be configured to sanitize training data. For the purposes of this disclosure, “sanitizing” training data is a process whereby training examples that interfere with convergence of a machine-learning model and/or process are removed to yield a useful result. For instance, and without limitation, a training example may include an input and/or output value that is an outlier from typically encountered values, such that a machine-learning algorithm using the training example will be skewed to an unlikely range of input 912 and/or output 908; a value that is more than a threshold number of standard deviations away from an average, mean, or expected value, for instance, may be eliminated. Alternatively, or additionally, one or more training examples may be identified as having poor-quality data, where “poor-quality” means having a signal-to-noise ratio below a threshold value. In one or more embodiments, sanitizing training data may include steps such as removing duplicative or otherwise redundant data, interpolating missing data, correcting data errors, standardizing data, identifying outliers, and/or the like. In one or more embodiments, sanitizing training data may include algorithms that identify duplicate entries or spell-check algorithms.

With continued reference to FIG. 9, in one or more embodiments, images used to train an image classifier or other machine-learning model and/or process that takes images as inputs 912 or generates images as outputs 908 may be rejected if image quality is below a threshold value. For instance, and without limitation, computing device, control unit 132, and/or machine-learning module 900 may perform blur detection. Elimination of one or more blurs may be performed, as a nonlimiting example, by taking Fourier transform or a Fast Fourier Transform (FFT) of image and analyzing a distribution of low and high frequencies in the resulting frequency-domain depiction of the image. Numbers of high-frequency values below a threshold level may indicate blurriness. As a further nonlimiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of image, based on the hypothesis that rapid changes indicate sharp edges in the image, and thus are indicative of a lower degree of blurriness. Blur detection may be performed using a wavelet-based operator, which uses coefficients of a discrete wavelet transform to describe the frequency and spatial content of images. Blur detection may be performed using statistics-based operators that take advantage of several image statistics as texture descriptors in order to compute a focus level. Blur detection may be performed by using discrete cosine transform (DCT) coefficients in order to compute a focus level of an image from its frequency content.

With continued reference to FIG. 9, computing device, control unit 132, and/or machine-learning module 900 may be configured to precondition one or more training examples. For instance, and without limitation, where a machine-learning model and/or process has one or more inputs 912 and/or outputs 908 requiring, transmitting, or receiving a certain number of bits, samples, or other units of data, one or more elements of training examples to be used as or compared to inputs 912 and/or outputs 908 may be modified to have such a number of units of data. In one or more embodiments, computing device, control unit 132, and/or machine-learning module 900 may convert a smaller number of units, such as in a low pixel count image, into a desired number of units by upsampling and interpolating. As a nonlimiting example, a low pixel count image may have 100 pixels, whereas a desired number of pixels may be 128. Control unit 132 may interpolate the low pixel count image to convert 100 pixels into 128 pixels. It should also be noted that one of ordinary skill in the art, upon reading the entirety of this disclosure, would recognize the various methods to interpolate a smaller number of data units such as samples, pixels, bits, or the like to a desired number of such units. In one or more embodiments, a set of interpolation rules may be trained by sets of highly detailed inputs 912 and/or outputs 908 and corresponding inputs 912 and/or outputs 908 downsampled to smaller numbers of units, and a neural network or another machine-learning model that is trained to predict interpolated pixel values using the training data 904. As a nonlimiting example, a sample input 912 and/or output 908, such as a sample picture, with sample-expanded data units (e.g., pixels added between the original pixels) may be input to a neural network or machine-learning model and output a pseudo replica sample picture with dummy values assigned to pixels between the original pixels based on a set of interpolation rules. As a nonlimiting example, in the context of an image classifier, a machine-learning model may have a set of interpolation rules trained by sets of highly detailed images and images that have been downsampled to smaller numbers of pixels, and a neural network or other machine-learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively, or additionally, computing device, control unit 132, and/or machine-learning module 900 may utilize sample expander methods, and/or a filter of any time (low-pass, high-pass, band-pass or any combination of multiple filters. For the purposes of this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, control unit 132, and/or machine-learning module 900 may use averaging, such as luma or chroma averaging in images, to fill in data units in between original data units.

With continued reference to FIG. 9, in one or more embodiments, computing device, control unit 132, and/or machine-learning module 900 may downsample elements of a training example to a desired lower number of data elements. As a nonlimiting example, a high pixel count image may contain 256 pixels, however a desired number of pixels may be 128. Control unit 132 may downsample the high pixel count image to convert 256 pixels into 128 pixels. In one or more embodiments, control unit 132 may be configured to perform downsampling on data. Downsampling, also known as decimation, may include removing every Nth entry in a sequence of samples, all but every Nth entry, or the like, which is a process known as “compression” and may be performed, for instance by an N-sample compressor implemented using hardware or software. Anti-aliasing and/or anti-imaging filters, and/or low-pass filters, may be used to eliminate side effects of compression.

With continued reference to FIG. 9, feature selection may include narrowing and/or filtering training data 904 to exclude features and/or elements, or training data including such elements that are not relevant to a purpose for which a trained machine-learning model and/or algorithm is being trained, and/or collection of features, elements, or training data including such elements based on relevance to or utility for an intended task or purpose for which a machine-learning model and/or algorithm is being trained. Feature selection may be implemented, without limitation, using any process described in this disclosure, including without limitation using training data classifiers, exclusion of outliers, or the like.

With continued reference to FIG. 9, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, wherein a difference between each value, X, and a minimum value, Xₘᵢₙ, in a set or subset of values is divided by a range of values, Xₘₐₓ−Xₘᵢₙ, in the set or subset:

$${X}_{n e w} = \frac{X - {X}_{min}}{{X}_{max} - {X}_{min}} .$$

Feature scaling may include mean normalization, wherein a difference between each value, X, and a mean value of a set and/or subset of values, Xₘₑₐₙ, is divided by a range of values, Xₘₐₓ−Xₘᵢₙ, in the set or subset:

$${X}_{n e w} = \frac{X - {X}_{m e a n}}{{X}_{max} - {X}_{min}} .$$

Feature scaling may include standardization, wherein a difference between X and Xₘₑₐₙ is divided by a standard deviation, σ, of a set or subset of values:

$${X}_{n e w} = \frac{X - {X}_{m e a n}}{σ} .$$

Feature scaling may be performed using a median value of a set or subset, X_{median}, and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

A Person of ordinary skill in the art, upon reviewing the entirety of this disclosure, will be aware of various alternative or additional approaches that may be used for feature scaling.

With continued reference to FIG. 9, computing device, control unit 132, and/or machine-learning module 900 may be configured to perform one or more processes of data augmentation. For the purposes of this disclosure, “data augmentation” is a process that adds data to a training data 904 using elements and/or entries already in the dataset. Data augmentation may be accomplished, without limitation, using interpolation, generation of modified copies of existing entries and/or examples, and/or one or more generative artificial intelligence (AI) processes, for instance using deep neural networks and/or generative adversarial networks. Generative processes may be referred to alternatively in this context as “data synthesis” and as creating “synthetic data”. Augmentation may include performing one or more transformations on data, such as geometric, color space, affine, brightness, cropping, and/or contrast transformations of images.

With continued reference to FIG. 9, machine-learning module 900 may be configured to perform a lazy learning process and/or protocol 920. For the purposes of this disclosure, a “lazy learning” process and/or protocol is a process whereby machine learning is conducted upon receipt of input 912 to be converted to output 908 by combining the input 912 and training data 904 to derive the algorithm to be used to produce the output 908 on demand. A lazy learning process may alternatively be referred to as a “lazy loading” or “call-when-needed” process and/or protocol. For instance, an initial set of simulations may be performed to cover an initial heuristic and/or “first guess” at an output 908 and/or relationship. As a nonlimiting example, an initial heuristic may include a ranking of associations between inputs 912 and elements of training data 904. Heuristic may include selecting some number of highest-ranking associations and/or training data 904 elements. Lazy learning may implement any suitable lazy learning algorithm, including without limitation a k-nearest neighbors algorithm, a lazy naive Bayes algorithm, or the like. A person of ordinary skill in the art, upon reviewing the entirety of this disclosure, will be aware of various lazy learning algorithms that may be applied to generate outputs as described in this disclosure, including without limitation lazy learning applications of machine-learning algorithms as described in further detail below.

With continued reference to FIG. 9, alternatively, or additionally, machine-learning processes as described in this disclosure may be used to generate machine-learning models 924. A “machine-learning model”, for the purposes of this disclosure, is a data structure representing and/or instantiating a mathematical and/or algorithmic representation of a relationship between inputs 912 and outputs 908, generated using any machine-learning process including without limitation any process described above, and stored in memory. An input 912 is submitted to a machine-learning model 924 once created, which generates an output 908 based on the relationship that was derived. For instance, and without limitation, a linear regression model, generated using a linear regression algorithm, may compute a linear combination of input data using coefficients derived during machine-learning processes to calculate an output datum. As a further nonlimiting example, a machine-learning model 924 may be generated by creating an artificial neural network, such as a convolutional neural network comprising an input layer of nodes, one or more intermediate layers, and an output layer of nodes. Connections between nodes may be created by “training” the network, in which elements from a training data 904 are applied to the input nodes, and a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning, as described in detail below.

With continued reference to FIG. 9, machine-learning module 900 may perform at least a supervised machine-learning process 928. For the purposes of this disclosure, a “supervised” machine-learning process is a process with algorithms that receive training data 904 relating one or more inputs 912 to one or more outputs 908, and seek to generate one or more data structures representing and/or instantiating one or more mathematical relations relating input 912 to output 908, where each of the one or more mathematical relations is optimal according to some criterion specified to the algorithm using some scoring function. For instance, a supervised learning algorithm may include inputs 912 described above as inputs, and outputs 908 described above as outputs, and a scoring function representing a desired form of relationship to be detected between inputs 912 and outputs 908. Scoring function may, for instance, seek to maximize the probability that a given input 912 and/or combination thereof is associated with a given output 908 to minimize the probability that a given input 912 is not associated with a given output 908. Scoring function may be expressed as a risk function representing an “expected loss” of an algorithm relating inputs 912 to outputs 908, where loss is computed as an error function representing a degree to which a prediction generated by the relation is incorrect when compared to a given input-output pair provided in training data 904. Supervised machine-learning processes may include classification algorithms as defined above. A person of ordinary skill in the art, upon reviewing the entirety of this disclosure, will be aware of various possible variations of at least a supervised machine-learning process 928 that may be used to determine a relation between inputs and outputs.

With continued reference to FIG. 9, training a supervised machine-learning process may include, without limitation, iteratively updating coefficients, biases, and weights based on an error function, expected loss, and/or risk function. For instance, an output 908 generated by a supervised machine-learning process 928 using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updates may be performed in neural networks using one or more back-propagation algorithms. Iterative and/or recursive updates to weights, biases, coefficients, or other parameters as described above may be performed until currently available training data 904 are exhausted and/or until a convergence test is passed. For the purposes of this disclosure, a “convergence test” is a test for a condition selected to indicate that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively, or additionally, one or more errors and/or error function values evaluated in training iterations may be compared to a threshold.

With continued reference to FIG. 9, a computing device, control unit 132, and/or machine-learning module 900 may be configured to perform method, method step, sequence of method steps, and/or algorithm described in reference to this figure, in any order and with any degree of repetition. For instance, computing device, control unit 132, and/or machine-learning module 900 may be configured to perform a single step, sequence, and/or algorithm repeatedly until a desired or commanded outcome is achieved; repetition of a step or a sequence of steps may be performed iteratively and/or recursively using outputs 908 of previous repetitions as inputs 912 to subsequent repetitions, aggregating inputs 912 and/or outputs 908 of repetitions to produce an aggregate result, reduction or decrement of one or more variables such as global variables, and/or division of a larger processing task into a set of iteratively addressed smaller processing tasks. A computing device, control unit 132, apparatus 100, or machine-learning module 900 may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. A person of ordinary skill in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 9, machine-learning process may include at least an unsupervised machine-learning process 932. For the purposes of this disclosure, an unsupervised machine-learning process is a process that derives inferences in datasets without regard to labels. As a result, an unsupervised machine-learning process 932 may be free to discover any structure, relationship, and/or correlation provided in the data. Unsupervised processes 932 may not require a response variable, may be used to find interesting patterns and/or inferences between variables, to determine a degree of correlation between two or more variables, or the like.

With continued reference to FIG. 9, machine-learning module 900 may be designed and configured to create machine-learning model 924 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include an clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to a person of ordinary skill in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, as will be apparent to a person of ordinary skill in the art upon reviewing the entirety of this disclosure.

With continued reference to FIG. 9, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

With continued reference to FIG. 9, a machine-learning model and/or process may be deployed or instantiated by incorporation into a program, apparatus, system, and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit, to represent a number according to any suitable encoding system including twos complement or the like, or may be stored in any volatile and/or non-volatile memory. Similarly, mathematical operations and input 912 and/or output 908 of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation application-specific integrated circuits (ASICs), production and/or configuration of reconfigurable hardware elements, circuits, and/or modules such as without limitation field programmable gate arrays (FPGAs), production and/or configuration of non-reconfigurable and/or non-rewritable memory elements, circuits, and/or modules such as without limitation non-rewritable read-only memory (ROM), other memory technology described in this disclosure, and/or production and/or configuration of any computing device and/or component thereof as described in this disclosure. Such deployed and/or instantiated machine-learning model and/or algorithm may receive inputs 912 from any other process, module, and/or component described in this disclosure, and produce outputs 908 to any other process, module, and/or component described in this disclosure.

With continued reference to FIG. 9, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively, or additionally, retraining, deployment, and/or instantiation may be event-based, and may be triggered, without limitation, by user inputs indicating sub-optimal or otherwise problematic performance and/or by automated field testing and/or auditing processes, which may compare outputs 908 of machine-learning models and/or algorithms, and/or errors and/or error functions thereof, to any thresholds, convergence tests, or the like, and/or may compare outputs 908 of processes described herein to similar thresholds, convergence tests or the like. Event-based retraining, deployment, and/or instantiation may alternatively, or additionally, be triggered by receipt and/or generation of one or more new training examples; a number of new training examples may be compared to a preconfigured threshold, where exceeding the preconfigured threshold may trigger retraining, deployment, and/or instantiation.

With continued reference to FIG. 9, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data 904 may include, without limitation, training examples including inputs 912 and correlated outputs 908 used, received, and/or generated from any version of any system, module, machine-learning model or algorithm, apparatus, and/or method described in this disclosure. Such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs 908 for training processes as described above. Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

With continued reference to FIG. 9, one or more processes or algorithms described above may be performed by at least a dedicated hardware unit 936. For the purposes of this disclosure, a “dedicated hardware unit” is a hardware component, circuit, or the like, aside from a principal control circuit and/or control unit 132 performing method steps as described in this disclosure, that is specifically designated or selected to perform one or more specific tasks and/or processes described in reference to this figure. Such specific tasks and/or processes may include without limitation preprocessing and/or sanitization of training data and/or training a machine-learning algorithm and/or model. Dedicated hardware unit 936 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously, in parallel, and/or the like. Such dedicated hardware units 936 may include, without limitation, graphical processing units (GPUs), dedicated signal processing modules, field programmable gate arrays (FPGA), other reconfigurable hardware that has been configured to instantiate parallel processing units for one or more specific tasks, or the like. Computing device, control unit 132, apparatus 100, or machine-learning module 900 may be configured to instruct one or more dedicated hardware units 936 to perform one or more operations described herein, such as evaluation of model and/or algorithm outputs, one-time or iterative updates to parameters, coefficients, weights, and/or biases, vector and/or matrix operations, and/or any other operations described in this disclosure.

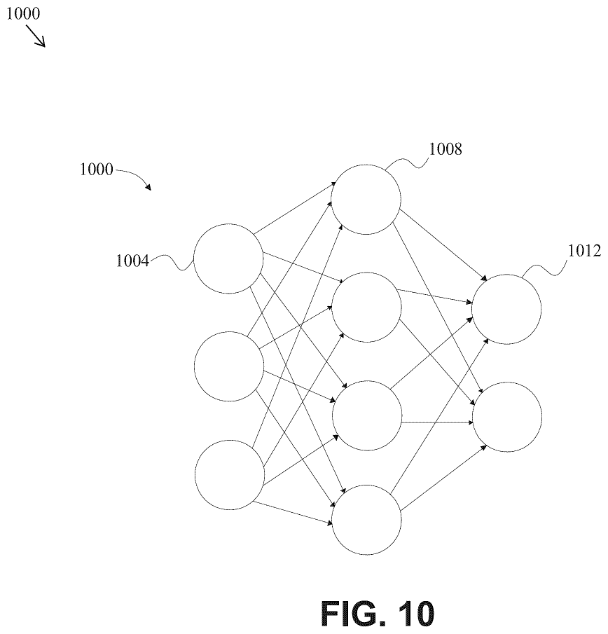

Referring now to FIG. 10, an exemplary embodiment of neural network 1000 is illustrated. For the purposes of this disclosure, a neural network or artificial neural network is a network of “nodes” or data structures having one or more inputs, one or more outputs, and a function determining outputs based on inputs. Such nodes may be organized in a network, such as without limitation a convolutional neural network, including an input layer of nodes 1004, at least an intermediate layer of nodes 1008, and an output layer of nodes 1012. Connections between nodes may be created via the process of “training” neural network 1000, in which elements from a training dataset are applied to the input nodes, and a suitable training algorithm (such as Levenberg-Marquardt, conjugate gradient, simulated annealing, or other algorithms) is then used to adjust the connections and weights between nodes in adjacent layers of the neural network 1000 to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network”. As a further nonlimiting example, neural network 1000 may include a convolutional neural network comprising an input layer of nodes 1004, one or more intermediate layers of nodes 1008, and an output layer of nodes 1012. For the purposes of this disclosure, a “convolutional neural network” is a type of neural network 1000 in which at least one hidden layer is a convolutional layer that convolves inputs to that layer with a subset of inputs known as a “kernel”, along with one or more additional layers such as pooling layers, fully connected layers, and the like.

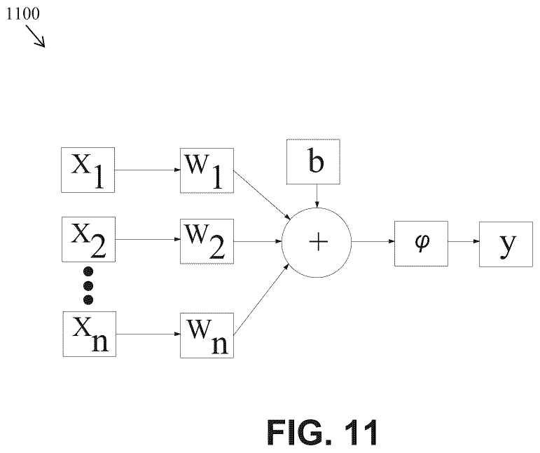

Referring now to FIG. 11, an exemplary embodiment of a node 1100 of neural network 1000 is illustrated. Node 1100 may include, without limitation, a plurality of inputs, xᵢ, that may receive numerical values from inputs to neural network 1000 containing the node 1100 and/or from other nodes 1100. Node 1100 may perform one or more activation functions to produce its output given one or more inputs, such as without limitation computing a binary step function comparing an input to a threshold value and outputting either a logic 1 or logic 0 output or its equivalent, a linear activation function whereby an output is directly proportional to input, and/or a nonlinear activation function wherein the output is not proportional to the input. Nonlinear activation functions may include, without limitation, a sigmoid function of the form

$$f ( x ) = \frac{1}{1 - {e}^{- x}}$$

given input x, a tanh (hyperbolic tangent) function of the form

$$\frac{{e}^{x} - {e}^{- x}}{{e}^{x} + {e}^{- x}} ,$$

a tanh derivative function such as f(x)=tanh²(x), a rectified linear unit function such as f(x)=max (0, x), a “leaky” and/or “parametric” rectified linear unit function such as f(x)=max (ax, x) for some value of a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid (x), a Gaussian error linear unit function such as f(x)=a(1+tanh (\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ, that may be used as activation functions. As a nonlimiting and illustrative example, node 1100 may perform a weighted sum of inputs using weights, wᵢ, that are multiplied by respective inputs, xᵢ. Additionally, or alternatively, a bias b may be added to the weighted sum of the inputs such that an offset is added to each unit in a neural network layer that is independent of the input to the layer. The weighted sum may then be input into a function, φ, which may generate one or more outputs, y. Weight, wᵢ, applied to an input, xᵢ, may indicate whether the input is “excitatory”, indicating that it has strong influence on the one or more outputs, y, for instance by the corresponding weight having a large numerical value, or “inhibitory”, indicating it has a weak influence on the one more outputs, y, for instance by the corresponding weight having a small numerical value. The values of weights, wᵢ, may be determined by training neural network 1000 using training data, which may be performed using any suitable process as described above.

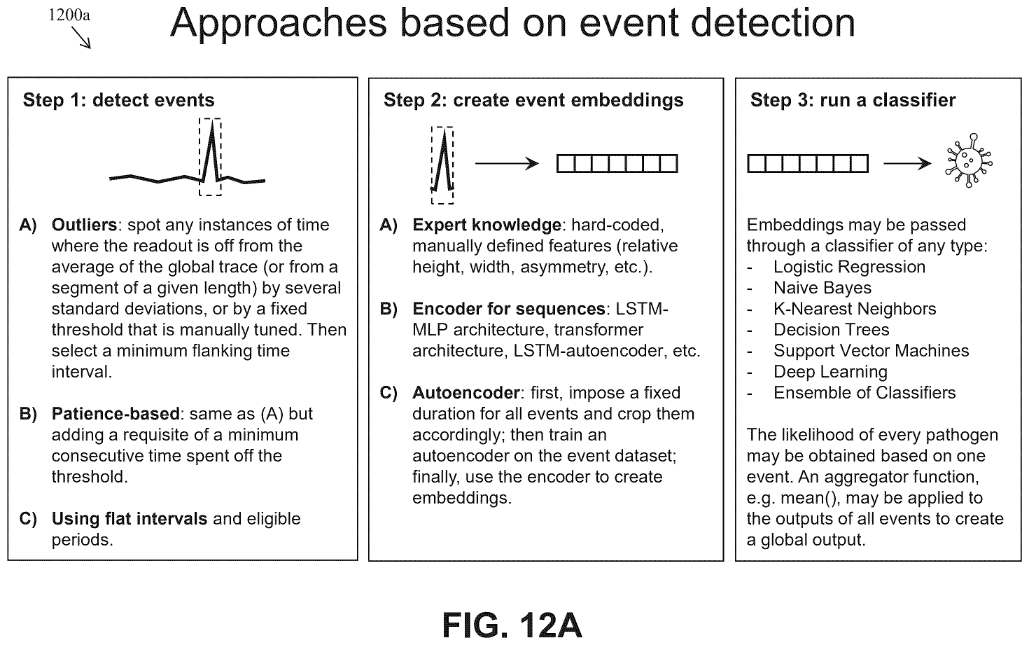

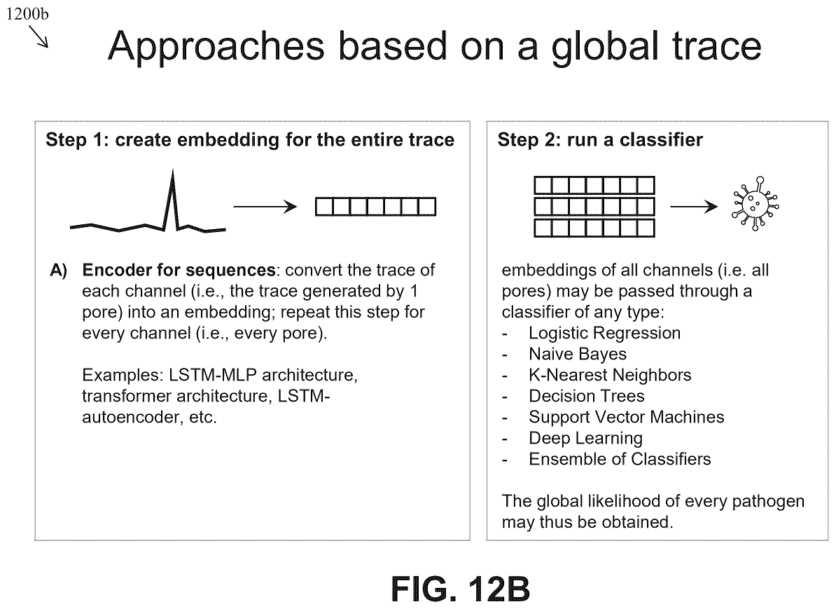

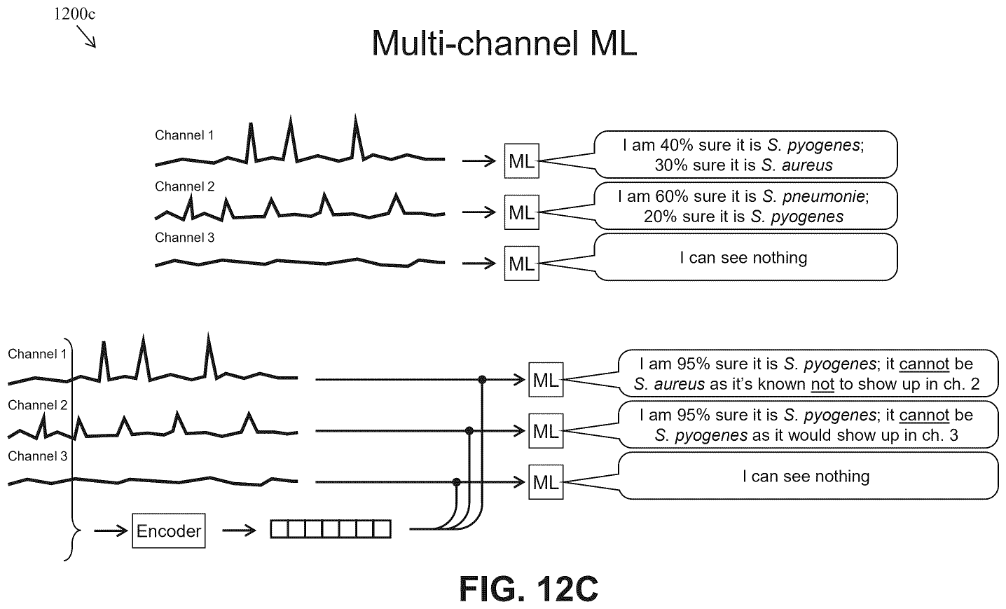

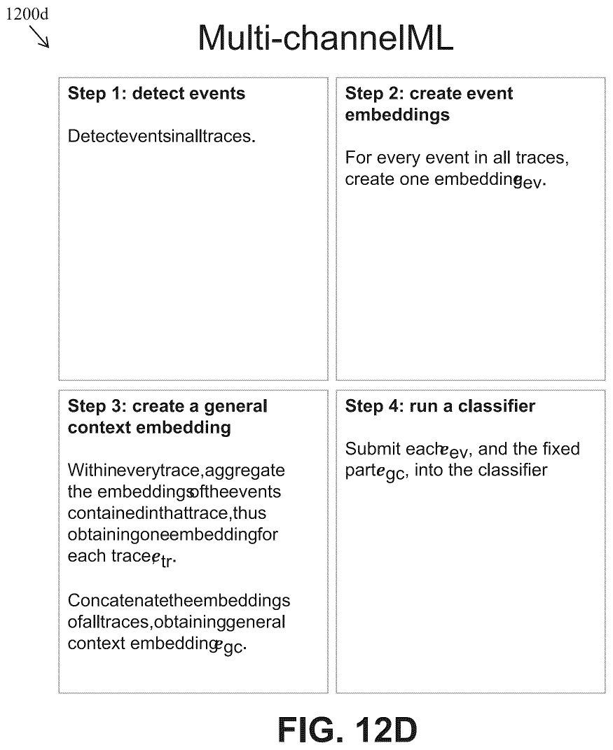

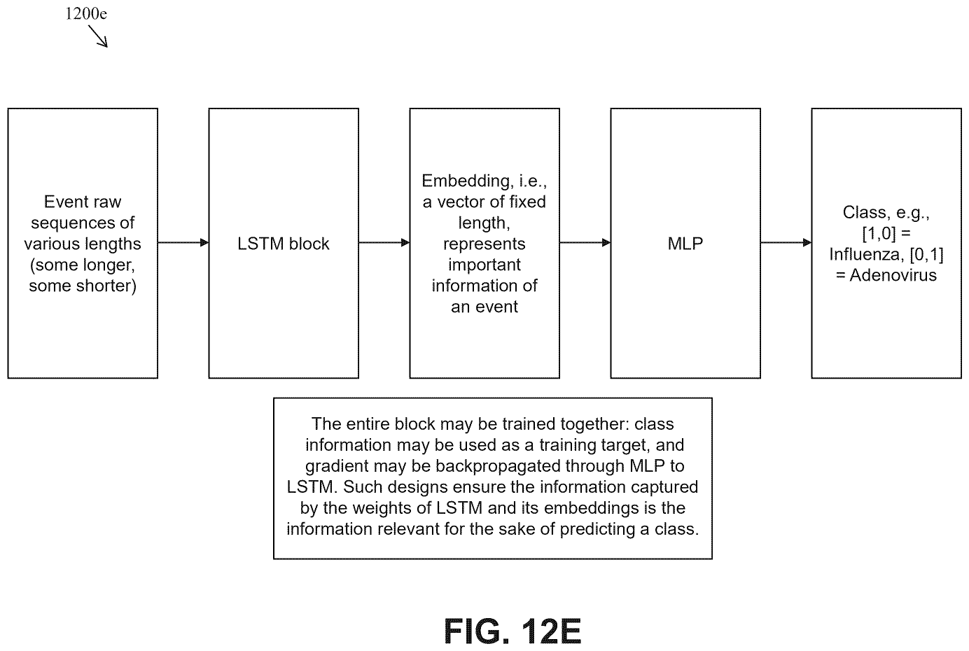

Referring now to FIGS. 12A-E, additional exemplary embodiments 1200a-e are illustrated to describe various possible workflows for implementing a machine-learning process in identification and/or quantification of a microbe 212. FIG. 12A illustrates an exemplary workflow pertaining to event detection, consistent with details described above in this disclosure. At step 1, apparatus 100 may be configured to detect events 516 within signal 500a. In some cases, step 1 may be implemented by spotting outliers, i.e., spotting any instances of time where a readout is off from the average the global trace (or from a segment therein of a given length) by several standard deviations, or by a fixed threshold that is manually tuned. A minimum flaking time interval may then be selected accordingly. Alternatively, in some cases, step 1 may include a patience-based approach. Such an approach may include a requisite of a given minimum consecutive time spent off the threshold. Alternatively, in some cases, step 1 may be implemented by selecting flat intervals and eligible periods, consistent with details described above pertaining to FIGS. 5A-C.

With continued reference to FIG. 12A, at step 2, apparatus 100 may be configured to create event embeddings. In some cases, event embeddings may be created based on expert knowledge. Such expert knowledge may include hard-coded, manually defined features including without limitation height including relative height, width, asymmetry, or the like. In some cases, event embeddings may be created using an encoder for sequences, such as without limitation an LSTM-MLP architecture, a transformer architecture, and/or an LSTM-autoencoder. In some cases, event embeddings may be created using an autoencoder. As a nonlimiting example, apparatus 100 may first impose a fixed duration for all events and crop them; accordingly, and an autoencoder may subsequently be trained on the event dataset and finally used to create embeddings.

With continued reference to FIG. 12A, at step 3, apparatus 100 may be configured to run a classifier. Embeddings generated from step 2 may be passed through any suitable type of classifier, such as without limitation a classifier based on logistic regression, a naive Bayes classifier, a k-nearest neighbors classifier, decision trees, support vector machines, XGBoost, and/or a deep-learning based classifier, among others. The likelihood of every microbe 212 or pathogen may be obtained based on one event. In some cases, a classifier used herein may include an ensemble of multiple classifiers or classification algorithms, which may be of the same type or of mixed types, so that each classifier/classification algorithm may independently vote for a class. This feature allows apparatus 100 to obtain a certainty score. For the purposes of this disclosure, a “certainty score” is a numerical indication that describes the certainty with which a microbe 212 may be identified based on a particular event 516. An aggregator function, e.g. mean( ) may be then applied to the outputs of all events to create a global output.

Referring now to FIG. 12B, FIG. 12B illustrates an exemplary workflow based on a global trace. At step 1, apparatus 100 may be configured to create embeddings for an entire trace. This step may be implemented using an encoder for sequences such as without limitation an LSTM-MLP architecture, a transformer architecture, and/or an LSTM-autoencoder. Specifically, apparatus 100 may convert the trace of each channel (i.e., the trace generated by one nanopore 104) into an embedding and repeat this step for every channel (i.e., every nanopore 104). At step 2, apparatus 100 may be configured to run a classifier. Embeddings generated from step 1 may be passed through any suitable type of classifier, such as without limitation a classifier based on logistic regression, a naive Bayes classifier, a k-nearest neighbors classifier, decision trees, support vector machines, a deep-learning based classifier, XGBoost, among others, consistent with details described above. In some cases, a classifier used herein may include an ensemble of multiple classifiers or classification algorithms, which may be of the same type or of mixed types, so that each classifier/classification algorithm may independently vote for a class. This feature allows apparatus 100 to obtain a certainty score, consistent with details described above. The global likelihood of every microbe 212 or pathogen may thus be obtained.

Referring now to FIG. 12C, FIG. 12C illustrates an exemplary workflow based on a multi-channel machine-learning model. In some cases, apparatus 100 may be configured to analyze each channel (i.e., the trace generated by each nanopore 104) independently. In some other cases, since some nanopores 104 may be selective for specific families of microbes 212, evaluation a single trace may be simplified if a machine-learning model factors in which other traces show events or not based on a pore multiplicity as described above. This step may be achieved by creating an encoder vector for a general context.

Referring now to FIG. 12D, FIG. 12D illustrates another exemplary workflow based on a multi-channel machine-learning model. At step 1, apparatus 100 may be configured to detect events in all traces, consistent with details described above. At step 2, apparatus 100 may be configured to create event embeddings. For every event in all traces, one embedding, eₑᵥ, may be created. At step 3, apparatus 100 may be configured to create a general context embedding. In some cases, specifically, within every trace, apparatus 100 may be configured to aggregate the embeddings of the events contained in that trace, thus obtaining one embedding for each trace, eₜᵣ. As nonlimiting examples, such aggregation step may be implemented by taking an average of these embeddings, using a mean( ) function, or alternatively, by applying an aggregator function. Apparatus 100 may then be configured to concatenate the embeddings of all traces to obtain a general context embedding e_{gc}. Alternatively, at step 3, trace-level global embeddings, as described in FIG. 12B, may be used as input. At step 4, apparatus 100 may be configured to run a classifier. Specifically, eₑᵥ and e_{gc} may be submitted to a classifier as inputs.

Referring now to FIG. 12E, FIG. 12E illustrates exemplary workflow of a classification machine-learning model in general. Event raw sequences may contain a variety of lengths, with some being longer or shorter than others. By passing these event raw sequences through an LSTM block, consistent with details described elsewhere in this disclosure, embeddings of a fixed length may be generated to represent important information of an event 516. These embeddings may be subsequently passed onto MLP, and corresponding class labels, such as [1,0] for Influenza vs. [0,1] for Adenovirus, may be generated. It is worth noting that this entire block may be trained together; class information may be used as a training target, and gradient may be backpropagated through MLP to LSTM. Such designs ensure the information captured by the weights of LSTM and its embeddings is the information relevant for the sake of predicting a class.

With continued reference to FIGS. 12A-E, apparatus 100 and/or one or more machine-learning models pertaining thereto may implement a transformer architecture. For the purposes of this disclosure, a “transformer architecture” is a neural network architecture that uses self-attention and positional encoding. A transformer architecture may be designed to process sequential input data or process the entire input all at once. For the purposes of this disclosure, “positional encoding” is a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In one or more embodiments, position vectors for a plurality of positions in a sequence may be assembled into a position matrix, wherein each row of position matrix may represent a position in the sequence. A transformer architecture may include an attention mechanism. For the purposes of this disclosure, an “attention mechanism” is a part of a neural network architecture that enables a system to dynamically quantify relevant features of the input data.

With continued reference to FIGS. 12A-E, several machine-learning techniques may allow apparatus 100 and/or one or more machine-learning models pertaining thereto to convert time sequences into embeddings. For the purposes of this disclosure, “embeddings” are one-dimensional (1D) vectors characterized by fixed dimensionality, low dimensionality and high expressivity. In some cases, such machine-learning techniques may include a transformer, consistent with details described above in this disclosure. In some cases, such machine-learning techniques may include recurrent neural networks (RNNs), consistent with details described above in this disclosure. In some cases, such machine-learning techniques may include long short-term memory (LSTM), consistent with details described above in this disclosure. In some cases, such machine-learning techniques may include gated recurrent units (GRUs). For the purposes of this disclosure, a “gated recurrent unit (GRU)” is a simplified version of LSTM that combines the forget and input gates into a single “update gate”. They have been shown to perform comparably to LSTMs on certain tasks. In some cases, such machine-learning techniques may include one-dimensional convolutional neural networks (1D-CNNs), consistent with details described above in this disclosure. In some cases, such machine-learning techniques may include temporal convolutional networks (TCNs), consistent with details described above in this disclosure.

With continued reference to FIGS. 12A-E, in one or more embodiments, apparatus 100 may be configured to create embeddings from raw data by following the pipeline below. At step 1, events 516 and their respective time sequences may be identified and highlighted, and events 516 may be filtered accordingly to ensure that they have a minimum duration and depth. At step 2, optionally, sequences may be normalized with respect to their respective baselines and padded with zeros before and/or after, so that their lengths become the same. At step 3, datasets of these sequences may be randomly divided into three subsets: a training subset, a validation subset, and a test subset, in proportions such as 60%-20%-20%, 80%-10%-10%, or the like. This step may be performed in a stratified manner to ensure that all classes of bacterial species are represented in equal proportions in training subset, validation subset, and test subset. At step 4, a machine-learning model of two layers may be created: a first layer with a machine learning architecture suitable to accept sequence data and output an embedding (e.g., LSTM, transformer, or the like) and a neural network second layer. Hyperparameters associated thereto may include an embedding size between 12 and 64, a single stacked layer, a minimum batch size between 16 and 128, and a sigmoid activation function, among others. These hyperparameters may be optimized with either a grid search or a random search. At step 5, the machine-learning model may be trained on padded and normalized sequences, using only training subset. The task may be a classification task targeting bacterial species of each event; the task may be a binary classification for two microbes 212, or a multi-class classification for three or more microbes 212. The loss function may include cross-entropy and/or binary cross-entropy. The optimizer may include ADAM. At each training iteration, performance is evaluated against validation subset, using early stop (i.e., patience); when, during training, performance keeps increasing in training subset, but stops increasing in validation subset, it may indicate that the machine-learning model is likely overfitting; to prevent this issue, when performance stops increasing in validation subset, a patience counter may start counting. If the performance starts to increase again, patience counter may be reset, and training may continue. If the performance in validation subset has never increased a single time, after a certain number of epochs (i.e., patience threshold), training may stop. The last epoch may be returned where an actual increase in validation is observed. At step 6, after training has ended, the machine-learning model may be used to output the embeddings from training, validation, test subsets. This is possible because LSTM layers create an embedding internally to be fed to the neural network trained conjointly with the LSTM, which sits downstream of the LSTM. Now an embedding (i.e., a vector of a length equal to the size of embedding) is present for every single event in a dataset. An embedding “summarizes” all the information contained in an event sequence, which may be useful with respect to the classification task on which the model is trained.

With continued reference to FIGS. 12A-E, following step 6, the next step may depend on the exact task to be performed. In some cases, if the task is to determine performance metrics during a validation step, predicted labels (i.e., microbial species such as bacteria) from test subset may be taken and compared to ground truth labels to compute an accuracy (i.e., [no. correctly classified cases/no. total cases]). If the task is to quantify the number of bacteria, such as without limitation for a substance sensitivity assay such as an antibiogram or the like, steps described above may be based on recordings from purified bacteria cultures. Then, sequences from mixed population may be taken and applied with the trained machine-learning model to predict their label. Prevalence ratios, for example and without limitation, [density of Escherichia coli/density of Salmonella enterica], may be determined at each step of a growth curve.

With continued reference to FIGS. 12A-E, to plot a PCA-PCA or a PCA-LDA correlation plot, as described above, embeddings from training subset (and, optionally, validation subset) may be taken to train a PCA model and/or or an LDA model. After such training, the prediction method of PCA or LDA may be ran on the embeddings from test subset as well to create dimensionally reduced 2D coordinates for all events in a dataset. Events may then be plotted based on such 2D coordinates.

With continued reference to FIGS. 12A-E, to calculate the probability of an event belonging to a certain class, a meta-machine-learning model (i.e., a decoder) may be trained. Embeddings from training and validation subsets may be taken and (optionally) concatenated with certain hard-coded features of an event, such as without limitation the event's width, relative height, AUC, asymmetry, number of peaks, etc. It is worth noting that, in some cases, an input for an encoder may include embeddings only, manually coded features only (i.e., hard-coded features), or a combination of embeddings and manually coded features. Such data may then be used to train a random forest model, or any other ensemble model that is not necessarily tree-based (e.g., XGBoost) but deemed suitable for multiple evaluators, by a person of ordinary skill in the art, upon reviewing the entirety of this disclosure. In some cases, such model may include an ensemble of any kind of classifiers that are capable of accepting tabular data, such as without limitation an ensemble of decision trees, an ensemble of a plurality of XGBoosts, an ensemble of a plurality of MLPs, and/or an ensemble of one or more decision trees combined with one or more XGBoosts and/or MLPs, among others. After training on training and validation subsets only, the machine-learning model may then be applied to all events in the training, validation, and test subsets, and specifically, and random forest model may evaluate how many trees voted for each class. For the purposes of this disclosure, a “tree” or “decision tree” is an individual unit in a random forest. Similarly, when an ensemble of classifiers is implemented, each unit within the ensemble may be an individual classifier. As a nonlimiting example, the percentage of trees in the random forest that voted for class A (e.g., Influenzavirus) may represent the probability of that event belonging to class A. It is worth noting that random forests may require a tabular input, and accordingly, in some cases, LSTMs, transformers, LSTM-autoencoders, or the like, may be necessary to create embeddings (i.e., vectors of fixed dimensionality) out of sequences of variable lengths. For the purposes of this disclosure, an “LSTM autoencoder” is an implementation of an autoencoder for sequence data using an LSTM architecture. Specifically, for a given dataset of sequences, such LSTM may be configured to read, encode, decode, and recreate an input sequence. The performance of an encoder-decoder LSTM may be evaluated based on its ability to recreate the input sequence. Once an encoder-decoder LSTM achieves a desired level of performance, the decoder part of the encoder-decoder LSTM may be removed, leaving just the encoder part. LSTM autoencoder may then be used to encode input sequences to a fixed-length vector. As a nonlimiting example, LSTM autoencoder may utilize two serial LSTM blocks. A first LSTM block may receive a sequence and transform it into an embedding, whereas a second LSTM block may use the embedding as input and transform it back to the original sequence. The two TSTM blocks combined and configured to identify one or more microbes 212. In doing so, an LSTM-autoencoder may create an embedding that captures a summarized version of the information that is contained in the sequence. The resulting vectors may then be used in a variety of applications, such as without limitation as a compressed representation of a sequence as an input to another supervised machine-learning model.

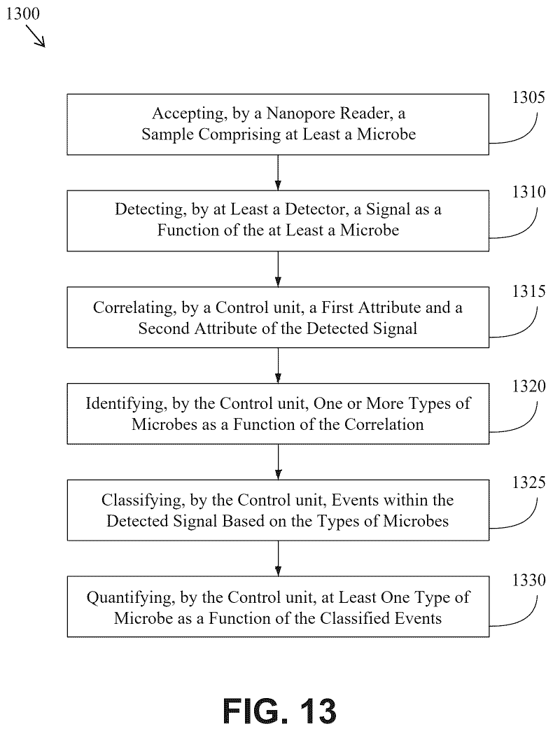

Referring now to FIG. 13, an exemplary embodiment of a method 1300 for simultaneous identification and quantification of a microbe is described. At step 1305, method 1300 includes accepting, by at least a nanopore reader 116, a sample including at least a microbe 212, wherein each nanopore reader 116 of the at least a nanopore reader 116 includes a plurality of flow cells 120a-n, wherein at least a flow cell 120a-n of the plurality of flow cells 120a-n is configured to accept a sample, and at least a detector 128 connected to the plurality of flow cells 120a-n, the at least a detector 128 configured to detect a signal 500a as a function of at least a translocated microbe 212 from the sample. This step may be implemented with reference to details described above in this disclosure and without limitation.

With continued reference to FIG. 13, at step 1310, method 1300 includes detecting, by at least a detector 128, signal 500a as a function of at least a microbe 212, wherein the at least a microbe 212 is translocated from first flow cell 120a-n to second flow cell 120a-n through at least a nanopore 104 of at least a nanopore 104. This step may be implemented with reference to details described above in this disclosure and without limitation.

With continued reference to FIG. 13, at step 1315, method 1300 includes correlating, by control unit 132, a first attribute and a second attribute of detected signal 500a. This step may be implemented with reference to details described above in this disclosure and without limitation.

With continued reference to FIG. 13, at step 1320, method 1300 includes identifying, by control unit 132, one or more types of microbes 212 as a function of the correlation. This step may be implemented with reference to details described above in this disclosure and without limitation.

With continued reference to FIG. 13, at step 1325, method 1300 includes classifying, by control unit 132, a plurality of events 516 within the detected signal 500a based on identified one or more types of microbes 212. This step may be implemented with reference to details described above in this disclosure and without limitation.

With continued reference to FIG. 13, at step 1330, method 1300 includes quantifying, by control unit 132, at least one type of microbe 212 of identified one or more types of microbes 212 as a function of the classified plurality of events 516. This step may be implemented with reference to details described above in this disclosure and without limitation.

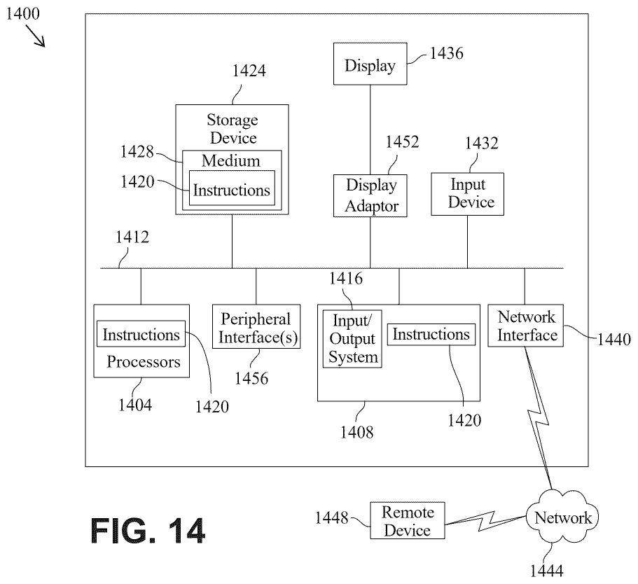

Referring now to FIG. 14, it is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to one of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module. Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission. Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein. Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

With continued reference to FIG. 14, the figure shows a diagrammatic representation of one embodiment of a computing device in the exemplary form of a computing system 1400 within which a set of instructions for causing the computing system 1400 to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computing system 1400 may include a processor 1404 and a memory 1408 that communicate with each other, and with other components, via a bus 1412. Bus 1412 may include any of several types of bus structures including, but not limited to, a memory bus, a memory controller, a peripheral bus, a local bus, and any combinations thereof, using any of a variety of bus architectures. Processor 1404 may include any suitable processor, such as without limitation a processor incorporating logical circuitry for performing arithmetic and logical operations, such as an arithmetic and logic unit, which may be regulated with a state machine and directed by operational inputs from memory and/or sensors; processor 1404 may be organized according to Von Neumann and/or Harvard architecture as a non-limiting example. Processor 1404 may include, incorporate, and/or be incorporated in, without limitation, a microcontroller, microprocessor, digital signal processor, field programmable gate array, complex programmable logic device, graphical processing unit, general-purpose graphical processing unit, tensor processing unit, analog or mixed signal processor, trusted platform module, a floating-point unit, and/or system on a chip.

With continued reference to FIG. 14, memory 1408 may include various components (e.g., machine-readable media) including, but not limited to, a random-access memory component, a read only component, and any combinations thereof. In one example, a basic input/output system 1416, including basic routines that help to transfer information between elements within computing system 1400, such as during start-up, may be stored in memory 1408. Memory 1408 (e.g., stored on one or more machine-readable media) may also include instructions (e.g., software) 1420 embodying any one or more of the aspects and/or methodologies of the present disclosure. In another example, memory 1408 may further include any number of program modules including, but not limited to, an operating system, one or more application programs, other program modules, program data, and any combinations thereof.

With continued reference to FIG. 14, computing system 1400 may also include a storage device 1424. Examples of a storage device (e.g., storage device 1424) include, but are not limited to, a hard disk drive, a magnetic disk drive, an optical disc drive in combination with an optical medium, a solid-state memory device, and any combinations thereof. Storage device 1424 may be connected to bus 1412 by an appropriate interface (not shown). Example interfaces include, but are not limited to, small computer system interface, advanced technology attachment, serial advanced technology attachment, universal serial bus, IEEE 1394 (FIREWIRE), and any combinations thereof. In one example, storage device 1424 (or one or more components thereof) may be removably interfaced with computing system 1400 (e.g., via an external port connector (not shown)). Particularly, storage device 1424 and an associated machine-readable medium 1428 may provide nonvolatile and/or volatile storage of machine-readable instructions, data structures, program modules, and/or other data for computing system 1400. In one example, software 1420 may reside, completely or partially, within machine-readable medium 1428. In another example, software 1420 may reside, completely or partially, within processor 1404.

With continued reference to FIG. 14, computing system 1400 may also include an input device 1432. In one example, a user of computing system 1400 may enter commands and/or other information into computing system 1400 via input device 1432. Examples of input device 1432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1432 may be interfaced to bus 1412 via any of a variety of interfaces (not shown) including, but not limited to, a serial interface, a parallel interface, a game port, a USB interface, a FIREWIRE interface, a direct interface to bus 1412, and any combinations thereof. Input device 1432 may include a touch screen interface that may be a part of or separate from display device 1436, discussed further below. Input device 1432 may be utilized as a user selection device for selecting one or more graphical representations in a graphical interface as described above.

With continued reference to FIG. 14, user may also input commands and/or other information to computing system 1400 via storage device 1424 (e.g., a removable disk drive, a flash drive, etc.) and/or network interface device 1440. A network interface device, such as network interface device 1440, may be utilized for connecting computing system 1400 to one or more of a variety of networks, such as network 1444, and one or more remote devices 1448 connected thereto. Examples of a network interface device include, but are not limited to, a network interface card (e.g., a mobile network interface card, a LAN card), a modem, and any combination thereof. Examples of a network include, but are not limited to, a wide-area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1444, may employ a wired and/or a wireless mode of communication. In general, any network topology may be used. Information (e.g., data, software 1420, etc.) may be communicated to and/or from computing system 1400 via network interface device 1440.

With continued reference to FIG. 14, computing system 1400 may further include a video display adapter 1452 for communicating a displayable image to a display device, such as display device 1436. Examples of a display device include, but are not limited to, a liquid crystal display (LCD), a cathode ray tube (CRT), a plasma display, a light emitting diode (LED) display, and any combinations thereof. Video display adapter 1452 and display device 1436 may be utilized in combination with processor 1404 to provide graphical representations of aspects of the present disclosure. In addition to a display device, computing system 1400 may include one or more other peripheral output devices including, but not limited to, an audio speaker, a printer, and any combinations thereof. Such peripheral output devices may be connected to bus 1412 via a peripheral interface 1456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method for simultaneous identification and quantification of one or more microbes, the method comprising:
accepting, by at least one nanopore reader, a sample comprising at least one microbe, wherein each nanopore reader of the at least one nanopore reader comprises:
a plurality of flow cells, wherein at least one flow cell of the plurality of flow cells is configured to accept the sample; and
at least one detector connected to the plurality of flow cells, the at least one detector configured to detect a signal as a function of at least one translocated microbe from the sample; and
detecting, by the at least one detector, a signal as a function of the at least one microbe, wherein the at least one microbe is translocated from a first flow cell to a second flow cell of the plurality of flow cells through at least one nanopore;
correlating, by a control unit, a first attribute and a second attribute of the detected signal;
identifying, by the control unit, two or more types of microbe as a function of the correlation;
classifying, by the control unit, a plurality of events within the detected signal based on the identified two or more types of microbe; and
quantifying, by the control unit, at least one type of microbe of the identified two or more types of microbe as a function of the classified plurality of events.

2. The method of claim 1, wherein:
the first flow cell of the plurality of flow cells is configured to accept the sample;
the second flow cell of the plurality of flow cells is configured to accept a reference;
the first flow cell intersects the second flow cell at a junction; and
the at least a nanopore is located at the junction and connects the first flow cell and the second flow cell.

3. The method of claim 1, wherein correlating the first attribute and the second attribute of the detected signal comprises:
receiving correlation training data comprising a plurality of exemplary correlations as outputs correlated to a plurality of exemplary signal attributes as inputs;
iteratively training a correlation machine-learning model using the correlation training data; and
correlating the first attribute and the second attribute of the detected signal using the trained correlation machine-learning model.

4. The method of claim 1, wherein classifying the plurality of events comprises classifying the plurality of events using a binary classification algorithm.

5. The method of claim 1, wherein classifying the plurality of events comprises classifying the plurality of events using a multi-class classification (MCC) algorithm.

6. The method of claim 1, wherein classifying the plurality of events comprises:
receiving classification training data comprising a plurality of exemplary classes as outputs correlated to a plurality of exemplary events as inputs;
iteratively training a classification machine-learning model using the classification training data; and
classifying the plurality of events using the trained classification machine-learning model.

7. The method of claim 6, wherein the plurality of exemplary events comprises events extracted from experimental data collected using one or more purified microbial samples.

8. The method of claim 6, wherein classifying the plurality of events further comprises:
determining, using the classification machine-learning model, a certainty score; and
filtering the plurality of events as a function of the certainty score.

9. The method of claim 6, wherein the classification machine-learning model comprises an ensemble of a plurality of classifiers.

10. The method of claim 1, wherein the detected signal comprises an optical signal.

11. The method of claim 1, wherein the detected signal comprises an electrical signal.

12. The method of claim 11, wherein the electrical signal comprises a resistive pulse.

13. The method of claim 1, wherein the at least a nanopore is excavated in a SiNₓ wafer, a silicon oxide wafer, a glass wafer, or a polyimide membrane, a graphene layer, a molybdenum disulfide (MoS₂) layer, a gallium arsenide (GaAs) wafer, an indium gallium arsenide (InGaAs) wafer, an indium phosphide (InP) wafer, a silicon carbide (SiC) wafer, a diamond-like carbon (DLC) wafer, an aluminum oxide (Al₂O₃) wafer, a titanium nitride (TiN) wafer, a titanium dioxide (TiO₂) wafer, a hafnium oxide (HfO₂), a zirconium oxide (ZrO₂) wafer, a boron nitride (BN) wafer, or a ceramic wafer.

14. The method of claim 1, wherein:
the at least a nanopore comprises at least a first nanopore and at least a second nanopore;
wherein the at least a first nanopore of the at least a nanopore has a first size between 100 nanometers and 20 micrometers; and
wherein the at least a second nanopore of the at least a nanopore has a second size between 100 nanometers and 20 micrometers; and
wherein the first size is different from the second size.

15. The method of claim 1, wherein:
the at least a nanopore comprises at least a first nanopore and at least a second nanopore;
wherein the at least a first nanopore of the at least a nanopore has a first geometry;
wherein the at least a second nanopore of the at least a nanopore has a second geometry; and
wherein the first geometry is different from the second geometry.

16. The method of claim 1, wherein the control unit is further configured to:
the at least a nanopore comprises at least a first nanopore and at least a second nanopore;
apply, on the at least a first nanopore of the at least a nanopore, a first voltage difference along a first longitudinal axis of the at least a first nanopore; and
apply, on the at least a second nanopore of the at least a nanopore, a second voltage difference along a second longitudinal axis of the at least a second nanopore, wherein the first voltage difference is different from the second voltage difference.

17. The method of claim 1, wherein the at least a nanopore comprises a coating layer.

18. The method of claim 1, wherein the at least a nanopore comprises a plurality of nanopores is disposed in a line.

19. The method of claim 1, wherein the at least a nanopore comprises a plurality of nanopores is disposed in a two-dimensional matrix or a three-dimensional matrix.

20. A method for simultaneous identification and quantification of one or more microbes, the method comprising:
accepting, by at least one nanopore reader, a sample comprising at least a microbe, wherein each nanopore reader of the at least one nanopore reader comprises:
a plurality of flow cells, wherein at least one flow cell of the plurality of flow cells is configured to accept the sample; and
at least one detector connected to the plurality of flow cells, the at least one detector configured to detect a signal as a function of at least one translocated microbe from the sample; and
detecting, by the at least one detector, a signal as a function of the at least a microbe, wherein the at least one microbe is translocated from a first flow cell to a second flow cell of the plurality of flow cells through a plurality of nanopores;
correlating, by a control unit, a first attribute and a second attribute of the detected signal;
identifying, by the control unit, two or more types of microbe as a function of the correlation;
classifying, by the control unit, a plurality of events within the detected signal based on the identified two or more types of microbe; and
quantifying, by the control unit, at least one type of microbe of the identified two or more types of microbe as a function of the classified plurality of events.

21. An apparatus for simultaneous identification and quantification of one or more microbes, the apparatus comprising:
at least a nanopore;
at least one nanopore reader, wherein each nanopore reader of the at least a nanopore reader comprises:
a plurality of flow cells, wherein at least one flow cell of the plurality of flow cells is configured to accept a sample; and
at least a detector connected to the plurality of flow cells, the at least a detector configured to detect a signal as a function of at least a translocated microbe from the sample; and
a control unit communicatively connected to the at least one detector, wherein the control unit is configured to:
correlate a first attribute and a second attribute of the detected signal;
identify two or more types of microbe as a function of the correlation;
classify a plurality of events within the detected signal based on the identified two or more types of microbe; and
quantify at least one type of microbe of the identified two or more types of microbe as a function of the classified plurality of events.

22. An apparatus for simultaneous identification and quantification of one or more microbes, the apparatus comprising:
a plurality of nanopores;
at least one nanopore reader, wherein each nanopore reader of the at least one nanopore reader comprises:
a plurality of flow cells, wherein at least a flow cell of the plurality of flow cells is configured to accept a sample; and
at least one detector connected to the plurality of flow cells, the at least one detector configured to detect a signal as a function of at least one translocated microbe from the sample; and
a control unit communicatively connected to the at least a detector, wherein the control unit is configured to:
correlate a first attribute and a second attribute of the detected signal;
identify two or more types of microbe as a function of the correlation;
classify a plurality of events within the detected signal based on the identified two or more types of microbe; and
quantify at least one type of microbe of the identified two or more types of microbe as a function of the classified plurality of events.
